(19) United States
(12) Patent Application Publication
(10) Pub. No.: US 2024/0124835 A1
(43) Pub. Date: Apr. 18, 2024

(54) METHODS AND DEVICES FOR GENERATING EMBRYOS IN VITRO FROM EMBRYONIC STEM CELLS

(71) Applicants: California Institute of Technology, Pasadena, CA (US); Cambridge Enterprise Limited, Cambridge (GB)

(72) Inventors: Magdalena Zernicka-Goetz, Pasadena, CA (US); Gianluca Amadei, Cambridge (GB); Charlotte Handford, Cambridge (GB)

(21) Appl. No.: 18/485,192

(22) Filed: Oct. 11, 2023

Related U.S. Application Data
(60) Provisional application No. 63/415,574, filed on Oct. 12, 2022.

(51) Int. Cl. C12N 5/073; C12M 1/00; C12M 1/34; C12M 3/00; C12M 3/04; C12N 15/85

(52) U.S. Cl. CPC C12N 5/0604; C12M 21/06; C12M 27/10; C12M 29/24; C12M 41/34; C12M 41/40; C12N 15/85; C12N 2500/32; C12N 2500/38; C12N 2501/115; C12N 2501/119; C12N 2501/30; C12N 2501/999; C12N 2800/106

(57) ABSTRACT

Disclosed herein include methods, compositions, culture media, and devices for in vitro culture of synthetic embryos from mammalian pluripotent stem cells. The in vitro embryo model is generated with embryonic and extraembryonic lineages derived from embryonic stem cells through transcription-factor-mediated reprogramming and can undergo advanced development to late headfold stages.

### RELATED APPLICATIONS

This application claims the benefit under 35 U.S.C. § 119(e) of U.S. Provisional Patent Application No. 63/415,574, filed Oct. 12, 2022, the content of which is incorporated herein by reference in its entirety for all purposes.

### STATEMENT REGARDING FEDERALLY SPONSORED R&D

This invention was made with government support under Grant No. HD104575 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365854-US-SeqList, created Sep. 20, 2023, which is 9,924 bytes in size. The information in the electronic format of the Sequence Listing is incorporated herein by reference in its entirety.

### BACKGROUND

### Field

The present disclosure relates generally to the field of cell culture, in particular, culturing embryos and stem cells.

### Description of the Related Art

Several in vitro models have been developed to recapitulate various events of post-implantation developments. Despite mimicking many aspects of early development, these systems fail to capture the entire complexity of signaling and morphological events along the complete body axes, largely due to the failure to recapitulate the spatiotemporal interplay of signaling pathways between embryonic and extraembryonic tissues. Consequently, these models do not represent complete embryonic structures and lack of the overall morphological resemblance to natural post-implantation mammalian embryos.

### SUMMARY

Disclosed herein includes a method of generating a synthetic embryo in vitro, the method comprising: (a) co-culturing a wild-type mammalian embryonic stem cell (ESC), a mammalian ESC overexpressing CDX2 transcription factor, and a mammalian ESC overexpressing GATA transcription factor under a condition allowing the ESCs to self-assemble into a gastrulating embryo structure; and (b) culturing the gastrulating embryo structure in a post-implantation culture medium under a condition allowing the gastrulating embryo structure to develop into a synthetic embryo. The GATA transcription factor can be, for example, GATA4. In some embodiments, the method further comprises: generating the mammalian ESC overexpressing GATA transcription factor and the mammalian ESC overexpressing CDX2 transcription factor by contacting a mammalian ESC carrying an inducible Gata gene and a mammalian ESC carrying an inducible Cdx2 gene with an inducer. In some embodiments, the inducer is doxycycline. In some embodiments, the method comprises contacting the mammalian ESC carrying the inducible Gata gene and the mammalian ESC carrying the inducible Cdx2 gene with the inducer is performed for a duration of about 2-10 hours, optionally about 6 hours. The ESCs can be, for example, co-cultured for up to 6 days. In some embodiments, the gastrulating embryo structure resembles an E6.0-E7.5 natural embryo structure, optionally an E6.0-E6.5, E6.5-E7.0, or E7.0-E7.5 natural embryo structure. In some embodiments, the ESCs are cultured in a substrate, optionally wherein the substrate comprises a dish, a U-plate, a flask or a microwell plate. In some embodiments, the ESCs are cultured in inverted pyramidal microwells. In some embodiments, each of the inverted-pyramidal microwells is about 400 μm or about 800 μm in size, optionally about 400 μm or about 800 μm diameter. In some embodiments, step (a) comprises culturing the ESCs in a feeder cell (FC) media, optionally passaging the ESCs in the feeder cell media at least two times. The ESCs can be, for example, cultured in the FC media for about 3 days.

In some embodiments, step (a) further comprises culturing the ESCs in an in vitro culture (IVC) media, optionally following culturing the ESCs in the FC media. The ESCs can be, for example, cultured in the IVC media for about 2 days. In some embodiments, the FC media and the IVC media comprise a basal culture medium. In some embodiments, the basal culture medium comprises Dulbecco's Modified Eagle Media (DMEM), DMEM Nutrient Mixture 12 (DMEM/F12), a non-human serum or serum substitute thereof, a reducing agent, an antibiotic, L-glutamine or an analogue thereof, or any combination thereof. In some embodiments, the non-human serum or serum substitute comprises fetal bovine serum, bovine serum albumin, KnockOut™ Serum Replacement, or any combination thereof. The reducing agent can comprise, for example, beta-mercaptoethanol (BME), N-acetyl-L-cysteine, dithiothreitol (DTT), or any combination thereof.

In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The FC media can, for example, comprise sodium pyruvate. In some embodiments, the FC media comprises non-essential amino acids. In some embodiments, the FC media comprises DMEM, fetal bovine serum, sodium pyruvate, GlutaMax, MEM non-essential amino acids, beta-mercaptoethanol, penicillin and/or streptomycin, or any combination thereof. In some embodiments, the FC media comprises DMEM, about 15% fetal bovine serum, about 1 mM sodium pyruvate, about 2 mM GlutaMax, about 1% MEM non-essential amino acids, about 0.1 mM beta-mercaptoethanol, about 1% penicillin and/or streptomycin, or any combination thereof. In some embodiments, the FC media further comprises an anticoagulant, optionally heparin, a fibroblast growth factor (FGF), optionally FGF2 and/or FGF4, or any combination thereof. In some embodiments, the FC media is supplemented with heparin, Fgf4, and/or ROCK inhibitor, optionally in the first day of the co-culturing.

In some embodiments, the IVC media comprises: a) insulin, an insulin analogue, or an insulin receptor agonist; b) estrogen, an estrogen analogue, or an estrogen receptor agonist; and c) progesterone, a progesterone analogue, or a progesterone receptor agonist. The insulin receptor agonist can be selected from IGF-I, IGF-II, analogues thereof, and any combination thereof. In some embodiments, the estrogen receptor agonist is selected from β-estradiol, estrone, estriol and estetrol, and any analogue thereof. In some embodiments, the IVC media comprises transferrin, sodium selenium, ethanolamine, or any analogue thereof. In some embodiments, the IVC media does not comprise sodium pyruvate. In some embodiments, the IVC media comprises DMEM/F12, fetal bovine serum, GlutaMax, ITS-X, β-estrodiol, progesterone, N-acetyl-L-cysteine, penicillin and/or streptomycin, or any combination thereof. In some embodiments, the IVC media comprises DMEM/F12, about 20% fetal bovine serum, about 2 mM GlutaMax, about 1×ITS-X, about 8 nM β-estrodiol, about 200 ng/ml progesterone, about 25 μM N-acetyl-L-cysteine, about 1% penicillin and/or streptomycin, or any combination thereof. In some embodiments, co-culturing the ESCs comprises increasing serum concentrations, optionally increasing the serum concentration from about 20% to about 30%, optionally when the ESCs are co-cultured in the IVC media.

In the method disclosed herein, step (a) can comprise transferring the ESCs from one substrate to another substrate. In some embodiments, step (a) is performed under a static condition. In some embodiments, 1 step (b) is for a duration of at least 3 days. In some embodiments, the post-implantation culture media is capable of supporting the development of embryo ex utero.

In some embodiments, the post-implantation culture media comprises DMEM, non-human serum, human cord serum, L-glutamine or an analogue thereof, antibiotics, or any combination thereof. In some embodiments, the non-human serums comprises rat and/or bovine serum. In some embodiments, the post-implantation culture medium comprises bicarbonate. In some embodiments, the post-implantation culture medium comprises HEPES. In some embodiments, the non-human serum is rat serum.

The post-implantation culture medium can comprise, for example, DMEM, rat serum, human cord serum, GlutaMax, penicillin and/or streptomycin, HEPES, or any combination thereof. In some embodiments, the post-implantation culture medium comprises about 25% DMEM, about 50% rat serum, and about 25% human cord serum. In some embodiments, step (b) comprises culturing the gastrulating embryo structure under a dynamic condition in a culture chamber, following culturing the gastrulating embryo structure under a static condition.

In some embodiments, culturing the gastrulating embryo structure under the static condition is for a duration of about 2 days. In some embodiments, culturing the gastrulating embryo structure under the dynamic condition is for a duration of at least one day. In some embodiments, the dynamic condition comprises suspension agitation, optionally rotation. In some embodiments, culturing the gastrulating embryo structure under the dynamic condition is performed in a rotating bottle culture chamber. In some embodiments, the rotating bottle culture chamber contains up to 3 synthetic embryoid in about 2 ml post-implantation medium. In some embodiments, step (b) comprises supplying the post-implantation culture medium with glucose, optionally with at least 3 mg/ml glucose, and optionally when culturing under a dynamic condition. In some embodiments, the synthetic embryo structure resembles an E8.0-E8.5 natural embryo structure. In some embodiments, the synthetic embryo structure is a neutralizing embryo structure. In some embodiments, the synthetic embryo has established headfolds, a beating heart, allantois, chorion structure, and/or yolk sac. In some embodiments, the synthetic embryo has developed amnion-like and yolk-sac-like membranes.

In some embodiments, the method does not comprise any in vivo step. In some embodiments, none of the wild-type ESC, the ESC overexpressing CDX2 transcription factor and the ESC overexpressing GATA transcription factor is present in an in vivo environment during step (a) or step (b); and optionally wherein the in vivo environment comprises a tissue, an organ, an organism, or a combination thereof. In some embodiments, the method does not comprise culturing an extra-embryonic stem cell, optionally, the extra-embryonic stem cell comprises an extra-embryonic trophoblast stem cell and/or an extra-embryonic endoderm stem cell. The synthetic embryo can be, for example, a mouse embryo.

Disclosed herein includes a synthetic embryo obtained by any of the method disclosed herein. Also disclosed herein includes an embryo culturing device. The device can comprise, for example, a rotating incubator comprising a culture medium; a plurality of gas cylinders; a gas mixer configured to receive gases from the plurality of gas cylinders; and a gas and pressure controller. In some embodiments, the plurality of gas cylinders comprise an oxygen cylinder, a nitrogen cylinder and a carbon dioxide cylinder. In some embodiments, the plurality of gas cylinders input the gases into the gas mixer at an input pressure of about 10 psi. In some embodiments, the gas mixer mixes the gases from the plurality of gas cylinders to generate a gas mixture and feeds the gas mixture to the rotating incubator, optionally the gas mixture is humidified in the gas mixer. In some embodiments, the pressure of the gas mixture at the entrance of the rotating incubator is about 0.5 psi. In some embodiments, the gas mixture exits the rotating incubator at a flow rate of about 8-10 bubbles/sec. In some embodiments, the rotating incubator is rolled at about 1-10 rpm, optionally at about 3 rpm. In some embodiments, the culture medium is replenished about every 24 hours. In some embodiments, the culture medium is a post-implantation culture medium.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1 depicts a non-limiting exemplary process of generating an in vitro mouse embryo model from embryonic stem cells. Upper panel: Induction of ESCs into extraembryonic lineages; Lower panel: EiTiX-embryoids recapitulate embryonic development to late headfold stages.

FIG. 2A-FIG. 2I depict non-limiting exemplary embodiments demonstrating that Cdr2-induced ESCs self-assembling with Gata4-induced ESCs and ESCs into post-implantation-like mouse embryoids. FIG. 2A depicts a schematic of the formation of cell aggregates in AggreWells. FIG. 2B and FIG. 2C depict Day 3 iCdx2 ESC aggregates showing elevated Eomes expression (FIG. 2B) and downregulated Oct4 (FIG. 2C) upon induction of Cdr2. FIG. 2D depicts a schematic of EiTiX-embryoid generation. In FIG. 2D, FBS is short for fetal bovine serum; KSR is short for knockout serum replacement. FIG. 2E depicts representative brightfield images of structures developing in AggreWells from Day 0 to Day 4. A structure resembling the early post-implantation mouse embryo can be seen on Day 4 in the well outlined in the box. FIG. 2F depicts all structures in the combined microwells from one AggreWell collected on Day 4 and stained to reveal Cdx2, Oct4, and Gata6, each of which was stained in the color indicated above the middle panel of FIG. 2F. Arrows in FIG. 2F indicate structures considered to exhibit correct organization. Such cylindrical structures with two cellular compartments and an epithelialized EPI-like cell layer (dashed outline in the left panel of FIG. 2F) were selected under brightfield microscopy. The efficiency of obtaining organized structures in EiTiX-embryoid system and ETiX-embryoid system is shown the right panel of FIG. 2F, in which . . . indicates p<0.001. Efficiency of ETiX-embryoid is taken from a previous publication. FIG. 2G depicts Day 4 EiTiX-embryoids stained to reveal Cdx2, Oct4, and Gata6, each of which was stained in the color indicated above FIG. 2G. FIG. 2H depicts Day 4 EiTiX-embryoid stained to reveal Cdx2, GFP, and Oct4, each of which was stained in the color indicated above each of the left three panels of FIG. 2H. The percentage of the Cdx2-positive cells that were also GFP-positive is shown in the right panel of FIG. 2H (n=49 structures). FIG. 1 depicts Day 4 EiTiX-embryoids stained to reveal Eomes, Ap2γ, and Oct4, each of which was stained in the color indicated above each panel of FIG. 2I. n=35/35 structures were positive for both Eomes and Ap2γ. All experiments were performed a minimum of 3 times. Scale bars, 10 μm, (FIG. 2B) and (FIG. 2C); 150 μm, (FIG. 2E) and (FIG. 2F); 50 μm, (FIG. 2G-FIG. 2I).

FIG. 3A-FIG. 3I depict non-limiting exemplary embodiments related to the post-implantation development of EiTiX-embryoids. The data demonstrates that EiTiX-embryoids establish an anterior-posterior axis and undergo gastrulation. FIG. 3A depicts a schematic showing the position of DVE/AVE in E5.5 and E6.5 mouse embryos. FIG. 3B depicts Day 4 and Day 5 EiTiX-embryoids stained to reveal Cer1-GFP, Oct4, and Dkk1, each of which was stained in the color indicated above each panel of FIG. 3B. The double-headed arrows indicate Dkk1-positive domains. FIG. 3C depicts localization of Cer1-GFP in Day 4 and Day 5 EiTiX-embryoids. The right panel of FIG. 3C shows statistics of n=57 Day 4 structures and 45 Day 5 structures. FIG. 3D depicts Day 5 EiTiX-embryoids stained to reveal T, Oct4, and Cer1-GFP, each of which was stained in the color indicated above each panel of FIG. 3D. Double-headed arrow indicates Cer1-GFP-expressing domain; white box outline indicates T- and Oct4-positive domain. FIG. 3E depicts percentages of Day 5 EiTiX-embryoids showing (1) expression of both Cer1-GFP and T in the same structure, (2) asymmetric Cer1-GFP expression, and (3) T expression on the opposite side from Cer1-GFP (n=42 structures). FIG. 3F depicts Day 5 and Day 6 EiTiX-embryoids stained to reveal T and Oct4 or Sox2, each of which was stained in the color indicated above each panel of FIG. 3F. The boxes enclose T-positive domain while dotted lines outline the structure and the lumen of EPI-like compartment (n=39/42 Day 5 structures and 32/32 Day 6 structures). FIG. 3G depicts percentage of T extension in Day 5 and Day 6 EiTiX-embryoids (n=39 Day 5 structures and 32 Day 6 structures; ****p<0.0001). FIG. 3H depicts Day 6 EiTiX-embryoid stained to reveal E-cadherin, N-cadherin, and T, each of which was stained in the color indicated above each panel of FIG. 3H. Dotted line indicates T- and N-cadherin-positive domain (n=14/21 structures with N-cadherin upregulation and E-cadherin downregulation from 4 experiments). FIG. 3I depicts schematic of EiTiX-embryoids using CAG-GFP iGata4 ESCs (with membrane GFP) and mTmG ESCs (with membrane tdTomato) to construct the VE-like layer and EPI-like compartment, respectively. Day 6 EiTiX-embryoid was stained to reveal GFP, RFP and Sox17, each of which was stained in the color indicated above each right two panels of FIG. 3I. Dotted line indicates the lumen of EPI-like compartment, while arrows mark definitive-endoderm-like cells intercalated into the VE-like layer (n=8/12 structures). All experiments were performed a minimum of 3 times. Scale bars indicate 50 μm, except in zoomed views, which indicate 15 μm.

FIG. 4A-FIG. 4J depict non-limiting exemplary embodiments related to the cell type composition of gastrulating EiTiX-embryoids in comparison to natural embryos. The data demonstrates that Day 6 EiTiX-embryoids capture major cell types of gastrulation. FIG. 4A depicts evaluation of cell state integrity and cell type composition of Day 6 EiTiX-embryoids using a recently established time-resolved model of mouse gastrulation. FIG. 4B depicts brightfield images of Day 6 EiTiX-embryoids (n=14, annotated as S #1-14) collected for single-structure, scRNA-seq, and overlaid with GFP expression (Cer1-GFP reporter in VE-like layer and membrane CAG-GFP from iCdx2 ESCs in ExE-like compartment). Arrow indicates Cer1-GFP expression (Scale bar, 100 μm). FIG. 4C depicts embryo-embryo cell type composition similarity matrix. Natural embryos were annotated based on embryonic age groups (the scale on the right not labeled as EiTiX); Day 6 EiTiX-embryoids appear in the color shown on the top of the scale on the right. FIG. 4D depicts cell type composition bars of individual Day 6 EiTiX-embryoids (left) and matched natural embryos (right, annotated according to the key shown below and on the right). FIG. 4E and FIG. 4H depict comparison of major embryonic germ layer frequency (FIG. 4E) and ExE lineage frequency (FIG. 4H) between Day 6 EiTiX-embryoids and matching natural embryos. Medians of frequencies were compared using a Wilcoxon-Mann-Whitney rank-sum test after down sampling of cell-state-specific cells to corresponding number of Day 6 EiTiX cell-state-specific cells (i.e. for each cell state individually). q values were calculated from p values according to the Benjamini-Hochberg procedure (ns, not significant; * indicates q<0.05). Major germ layers include embryonic ectoderm (e.g., forebrain/midbrain/hindbrain, rostral neural plate, surface ectoderm, caudal neural plate, and definitive ectoderm), embryonic endoderm (e.g., definitive endoderm, gut, hindgut, and visceral and anterior visceral endoderm), embryonic mesoderm (e.g., tail bud-, early and late nascent-, caudal-, presomitic-, somitic-, paraxial-, rostral-, cardioparyngeal-, and lateral and intermediate-mesoderm), ExE mesoderm (e.g., amnion/chorion progenitor, amnion/chorion, allantois, and ExE mesoderm), EPC-lineage (e.g., SpT-Gly, TGC progenitors, uncommitted EPC, pTGC, and SpA-TGCs), and chorion-lineage (e.g., intermediate ExE, chorion progenitors, and chorion). FIG. 4F and FIG. 4I depict pooled embryonic (FIG. 4F) and ExE (FIG. 4I) cell type frequencies comparison between Day 6 EiTiX-embryoids and matched natural embryos. FIG. 4G and FIG. 4J depict bulk differential gene expression per cell type of Day 6 EiTiX cells against matched embryo cells in embryonic cell types (FIG. 4G, EPI and primitive streak) and ExE cell types (FIG. 4J, chorion progenitors and chorion). In FIG. 4G and FIG. 4J, dots represent individual genes. Color annotated dots mark genes with a 2-fold change in expression (dark dots in the top left section outlined by dashed line: above 2-fold decrease in Day 6 EiTiX cells; dark dots in the bottom right section outlined by dashed line: above 2-fold increase in Day 6 EiTiX cells).

FIG. 5A-FIG. 5L depict non-limiting exemplary embodiments and data related to the development of EiTiX-embryoids in post-implantation culture medium. The data demonstrates that EiTiX-embryoids develop to late headfold stages with heart and chorion development. FIG. 5A depicts a schematic showing culture conditions of EiTiX-embryoids to Day 8 (FBS, fetal bovine serum; RS, rat serum; HCS, human cord serum). FIG. 5B depicts representative brightfield images of EiTiX-embryoids cultured from Day 5 to Day 8 and E6.5 natural embryo cultured in vitro for 3 days (Al, allantois; Ch, chorion; H, heart; HF, headfolds; T, tail; and scale bar, 100 μm). FIG. 5C depicts brightfield images of Day 7 and Day 8 EiTiX-embryoids before dissection of yolk-sac-like membrane (Am, amnion; BI, blood island; YS, yolk sac. Scale bar, 200 μm (Day 7); 500 μm (Day 8)). FIG. 5D depicts efficiency of EiTiX-embryoid and ETiX-embryoid progression from Day 4 to 5, Day 5 to 6, Day 6 to 7, and Day 7 to 8. Efficiency of ETiX-embryoid is taken from a recent publication. Embryoids were selected on each day for further culture and experiments were performed a minimum of 3 times. Multiple t tests were performed. The term ns in FIG. 5D is short for non-significant. FIG. 5E depicts a schematic showing major cell types in E8.0 and E8.5 embryos. FIG. 5F depicts dorsal view of Day 7 EiTiX-embryoid and E6.5 natural embryo cultured in vitro for 2 days and stained to reveal Sox1 and Sox2, each of which was stained in the color indicated above each of the two panels of FIG. 5F (Scale bar, 200 μm). FIG. 5G depicts lateral view of Day 7 and Day 8 EiTiX-embryoids stained to reveal neuroepithelial markers Sox1 or Sox2 and heart markers Myh2 or Gata4, each of which was stained in the color indicated above each of the two panels of FIG. 5G (scale bar, 200 μm). FIG. 5H depicts ventral view of Day 8 EiTiX-embryoid stained to reveal Sox2 and Gata4, each of which was stained in the color indicated above FIG. 5H (scale bar, 200 μm), resembling the linear heart tube stage. FIG. 5I depicts lateral view of Day 7 EiTiX-embryoid and E6.5 natural embryo cultured in vitro for 2 days stained to reveal heart marker Gata4, pharyngeal mesoderm marker Isl1, and forebrain marker Otx2, each of which was stained in the color indicated on the left of FIG. 5H (scale bar, 200 μm). FIG. 5J depicts lateral view of Day 8 EiTiX-embryoids stained to reveal Sox2 and T, each of which was stained in the color indicated on the left of FIG. 5J. Magnified panels show co-expression in tail region (in the box). In FIG. 5J, the scale bar is 200 μm in the left panel and 100 μm (zoomed) in the two right panels. FIG. 5K depicts representative brightfield and GFP fluorescence image of Day 5 to 8 EiTiX-embryoids to track the contribution of CAG-GFP iCdx2 ESCs (scale bar, 50 μm (Day 5); 200 μm (Day 6); 500 μm (Day 8)). Structures show GFP expression in chorion-like region. FIG. 5L depicts dissected chorionic structure from Day 8 EiTiX-embryoid stained to reveal GFP, Hand1, and Keratin18, each of which was stained in the color indicated on the left of each panel of FIG. 5L (scale bar, 100 μm).

FIG. 6 depicts non-limiting exemplary photos taken at several different time points showing the beating heart region of Day 8 EiTiX-embryoids in one example, related to FIG. 5A-FIG. 5L.

FIG. 7 depicts non-limiting exemplary photos taken at several different time points showing the beating heart region of Day 8 EiTiX-embryoids in another example, related to FIG. 5A-FIG. 5L.

FIG. 8A-FIG. 8J depict non-limiting exemplary embodiments and data related to cell state and composition analysis of neurulating embryoids using scRNA-seq. FIG. 8A depicts brightfield images of Day 8 EiTiX-embryoids collected for single-structure, scRNA-seq. The yolk sac-like membrane was partially opened to reveal embryonic structures (Al, allantois; H, heart; HF, headfolds; T, tail; and scale bar, 500 μm). FIG. 8B depicts embryo-embryo cell type composition similarity matrix. Natural embryos are annotated based on embryonic age groups (the scale on the right not labeled as EiTiX); Day 8 EiTiX-embryoids appear in the color shown on the top of the scale on the right. FIG. 8C depicts cell type composition bars of individual Day 8 EiTiX-embryoids (left) and matched natural embryos (right, annotated according to the legend on the right of the bar charts). FIG. 8D and FIG. 8G depict pooled embryonic (FIG. 8D) and ExE (FIG. 8G) cell type frequencies comparison between Day 8 EiTiX-embryoids and matched natural embryos. FIG. 8E and FIG. 8H depict bulk differential gene expression per cell state of Day 6 EiTiX cells against matched embryo cells in embryonic cell type (FIG. 8E, cardiomyocyte) and ExE cell type (FIG. 8H, chorion). Dots represent individual genes. Color annotated dots mark genes with a 2-fold change in expression (dark dots in the top left section outlined by dashed line: above 2-fold decrease in Day 6 EiTiX cells; dark dots in the bottom right section outlined by dashed line: above 2-fold increase in Day 6 EiTiX cells). FIG. 8F depicts GFP channel bimodal distribution (left) with threshold use to define GFP-positive cell population shown as dots, annotated according to cell type (right). FIG. 8I depicts Day 5 EiTiX-embryoids and E6.5 natural embryos stained to reveal trophoblast stem markers Sox2, Cdx2, and Eomes, each of which was stained in the color indicated above each panel of FIG. 8I (scale bar, 50 μm). Brackets show the absence of trophoblast stem markers in the tip of ExE in E6.5 embryos. FIG. 8J depicts quantification of the extent of ExE expression of Sox2. It was determined by dividing the height of the expression domain (bracket on the left) by the height of ExE (bracket on the right), multiplied by 100% (n=19 E6.5 embryos from 2 experiments and 78 Day 5 EiTiX-embryoids from 3 experiments; **** p<0.0001; and scale bar, 50 μm).

FIG. 9A-FIG. 9M depict non-limiting exemplary characterization of Cdx2 inducible cells and Day 4 EiTiX-embryoids, related to FIG. 2A-FIG. 2I. FIG. 9A depicts fold changes of Cdx2 mRNA level in four different clones of iCdx2 ESCs 6 hours after Dox induction, determined by qRT-PCR. Student's t-test compares fold change with respect to uninduced counterparts. FIG. 9B depicts relative Cdx2 mRNA levels in uninduced iCdx2 ESCs, 6-hour induced CAG iCdx2 ESCs, and TSCs as compared to unmodified ESCs. FIG. 9C depicts immunofluorescence images of Cdx2 expression in uninduced and 6-hour induced iCdx2 ESCs. Percentage of Cdx2⁺ cells is shown in the bottom panel. In FIG. 9C, n=3 experiments, 5-10 random fields imaged for each condition in each experiment; and scale bar is 50 g m. FIG. 9D-FIG. 9E depict percentage of Eomes⁺ (FIG. 9D) and Oct4+(FIG. 9E) cells in Day 3 aggregates of uninduced, induced iCdx2 ESCs and TSCs. In FIG. 9D, n=43 uninduced aggregates, 50 induced aggregates and 39 TSC aggregates. In FIG. 9E, n=40 uninduced aggregates, 40 induced aggregates and 43 TSC aggregates. FIG. 9F-FIG. 9H depict relative mRNA levels of TSC markers Elf5 (FIG. 9F), Eomes (FIG. 9G) and Gaa3 (FIG. 9H) in Day 3 aggregates, normalized to mRNA levels in unmodified ESC aggregates. FIG. 9I depicts Day 4 structures generated using the ETiX embryo protocol, stained to reveal Cdx2, Oct4 and Gata6, each of which was stained in the color indicated on the left of FIG. 9I (scale bar, 150 μm). Arrow indicates structure with correct organization. Graph in the right panel shows percentages of structures with Cdx2-positive cells, with cells from all three lineages and with correct organization. FIG. 9J-FIG. 9M depict size comparison of E5.5 mouse embryos, Day 4 EiTiX-embryoids and Day 4 ETiX embryos in terms of height (FIG. 9J), ExE/iTS/TS height (FIG. 9K), Epi/ES height (FIG. 9L) and width (FIG. 9M) (n=6 E5.5 mouse embryos, 46 Day 4 EiTiX-embryoids and 15 Day 4 ETiX embryos). All experiments were performed minimum 3 times with the exception of (FIG. 9I). Statistic tests used are Student's t-test in FIG. 9A and FIG. 9C, and one-way ANOVA followed by Bonferroni's multiple comparisons test was performed in FIG. 9B, FIG. 9D-FIG. 9H and FIG. 9J-FIG. 9M (*p<0.05; **p<0.01; ***p<0.001; ****p<0.0001; ns, non-significant).

FIG. 10A-FIG. 10I depict non-limiting exemplary embodiments and data related to EiTiX-embryoids establishing an anterior-posterior axis and undergoing gastrulation, related to FIG. 3A-FIG. 3I. FIG. 10A depicts Day 4 and Day 5 EiTiX-embryoids stained to reveal Cer1-GFP, Oct4 and Lefty1, each of which was stained in the color indicated on the top of each panel of FIG. 10A. Arrows indicate the Lefty1-positive domain. FIG. 10B depicts percentages of EiTiX-embryoids expressing different combinations of AVE markers (n=29-32 Day 4 EiTiX-embryoids and 18-28 Day 5 EiTiX-embryoids). FIG. 10C depicts localization of Dkk1 and Lefty1 in Day 4 and Day 5 EiTiX-embryoids (Dkk1: n=29 Day 4 EiTiX-embryoids, 18 Day 5 EiTiX-embryoids; Lefty1: n=30 Day 4 EiTiX-embryoids, 22 Day 5 EiTiX-embryoids). FIG. 10D depicts percentages of EiTiX-embryoids expressing different combinations of anterior/posterior markers (n=42 Day 5 EiTiX-embryoids (Cer1-GFP⁺/T⁺), 39 Day 5 EiTiX-embryoids (Cer1⁺/Eomes⁺), 37 Day 5 EiTiX-embryoids (Dkk1⁺/Eomes⁺) and 38 Day 5 EiTiX-embryoids (Lefty1⁺/Eomes⁺)). FIG. 10E depicts Day 5 EiTiX-embryoids stained for Eomes, Oct4 and one of Cer1, Dkk1 and Lefty1, each of which was stained in the color indicated on the top of each panel of FIG. 10E. Arrows indicate Dkk1- or Cer1- or Lefty1-positive domain, while the box indicates Dkk1- or Cer1- or Lefty1- and Eomes-double positive domains. FIG. 10F depicts graph shows percentages of Day 5 EiTiX-embryoids with 1) AVE marker and Eomes expression, 2) asymmetric AVE marker expression, and 3) Eomes expression on the opposite side to AVE (n=114 EiTiX-embryoids). FIG. 10G depicts Day 6 EiTiX-embryoid stained to reveal T and Foxa2, each of which was stained in the color indicated on the top of each panel of FIG. 10G (n=15/16 EiTiX-embryoids with T- and Foxa2-double positive cells). Box indicates T- and Foxa2-double positive domain; dotted lines indicate lumen of ES compartment. FIG. 10H depicts Day 6 EiTiX-embryoid stained to reveal Foxa2 and Sox17, each of which is stained in the color indicated on the top of each panel of FIG. 10H (n=15/17 EiTiX-embryoids with Foxa2- and Sox17-double positive cells). Box indicates Foxa2- and Sox17-double positive domain; and dotted lines indicates lumen of ES compartment. FIG. 10I depicts whole mount in situ hybridization to reveal Bmp4 in Day 5 and Day 6 EiTiX-embryoids (n=9/10 Day 5 EiTiX-embryoids and 13/15 Day 6 EiTiX-embryoids). All experiments were performed minimum 2 times. Scale bars are 50 μm, except in zoomed views, which are 15 μm.

FIG. 11A-FIG. 11D depict non-limiting exemplary embodiments and data related to Day 6 EiTiX-embryoids capturing major cell types of gastrulation, related to FIG. 4A-FIG. 4J. FIG. 11A depicts developmental time (Eₜ) over embryo rank, annotated by age group (in 1Eₜ-1.5Eₜ intervals, legend in FIG. 4C). FIG. 11B depicts Day 6 and 8 EiTiX combined manifold, single cells (small dots) and Metacells (big dots) annotated by cell state (legend in FIG. 11C). Right panels of FIG. 11B show manifold of matched natural embryos and projection of pooled Day 6 EiTiX-embryoids on the manifold. FIG. 11C depicts pooled ExE (top) and embryonic (bottom) cell-state frequencies of Day 6 EiTiX-embryoids (left panel) and time-matched natural embryos (right panel). FIG. 11D depicts bulk differential gene expression per cell state of Day 6 EiTiX cells against matched embryo cells; early nascent mesoderm (left) and anterior primitive streak (right). Dots represent individual genes. Color annotated dots mark genes with a two-fold change in expression (dark dots in the top left section outlined by dashed line—above two-fold decrease in Day 6 EiTiX cells, dark dots in the bottom right section outlined by dashed line—above two-fold increase in Day 6 EiTiX cells).

FIG. 12A-FIG. 12E depict non-limiting exemplary embodiments and data related to the development of EiTiX-embryoids to late headfold stages with heart and chorion development (related to FIG. 5A-FIG. 5L). FIG. 12A-FIG. 12B depict examples of DAPI stained Day 8 EiTiX-embryoids (FIG. 12A) and underdeveloped Day 8 structures (FIG. 12B). Underdeveloped Day 8 structures showed stunted overall development (panel 1 in FIG. 12B) or impaired axial elongation to generate posterior structures (panels 2-4 in FIG. 12B). In FIG. 12A, H indicates heart; HF indicates headfolds; and T indicates tail. The scale bars in FIG. 12A-FIG. 12B are 200 μm. FIG. 12C depicts lateral view of Day 8 EiTiX-embryoid stained to reveal heart marker Gata4, pharyngeal mesoderm marker Isl1, and forebrain marker Otx2, each of which was stained in the color indicated on the top of each panel of FIG. 12C (Scale bar, 200 μm). FIG. 12D depicts Day 6 EiTiX-embryoid stained to reveal Runx1 and T, each of which was stained in the color indicated on the top of FIG. 12D. Arrow in FIG. 12D indicates Runx1- and T-double positive cells. In FIG. 12D, n=3/4 EiTiX-embryoids with Runx1- and T-double positive cells. Scale bar in the left panel of FIG. 12D is 100 μm, while the scale bar in the right panels is 20 μm (zoomed). FIG. 12E depicts dissected yolk sac-like membrane from Day 8 EiTiX-embryoid stained to reveal Runx1 (stained in the color indicated on the top of FIG. 12E) (n=6/6 EiTiX-embryoids with Runx1 expression; scale bar, 100 μm).

FIG. 13A-FIG. 13D depict non-limiting exemplary embodiments and data related to cell state and composition analysis of neurulating embryoids using scRNA-seq, related to FIG. 8A-FIG. 8J. FIG. 13A depicts pooled ExE (left bar) and embryonic (right bar) cell-state frequencies of Day 8 EiTiX structures (top panel) and time-matched natural embryos (bottom panel, annotated according to the legend on the right). FIG. 13B depicts manifold of matched natural embryos and projection of individual Day 8 EiTiX-embryoids on the manifold (legend in FIG. 13A). FIG. 13C depicts bulk differential gene expression per cell state of Day 8 EiTiX cells against matched embryo cells (e.g., extraembryonic mesoderm, allantois, amnion/chorion, cardiopharyngeal mesoderm, forebrain/midbrain/hindbrain, hindgut and erythroid 3). Dots represent individual genes. Color annotated dots mark genes with a two-fold change in expression (dark dots in the top left section outlined by dashed line—above two-fold decrease in Day 8 EiTiX cells, dark dots in the bottom right section outlined by dashed line—above two-fold increase in Day 8 EiTiX cells). FIG. 13D depicts quantification of the extent of ExE expression Cdx2 and Eomes in E6.5 embryos and Day 5 EiTiX-embryoids (n=19 E6.5 embryos from 2 experiments and 78 Day 5 EiTiX-embryoids from 3 experiments; **** p<0.0001).

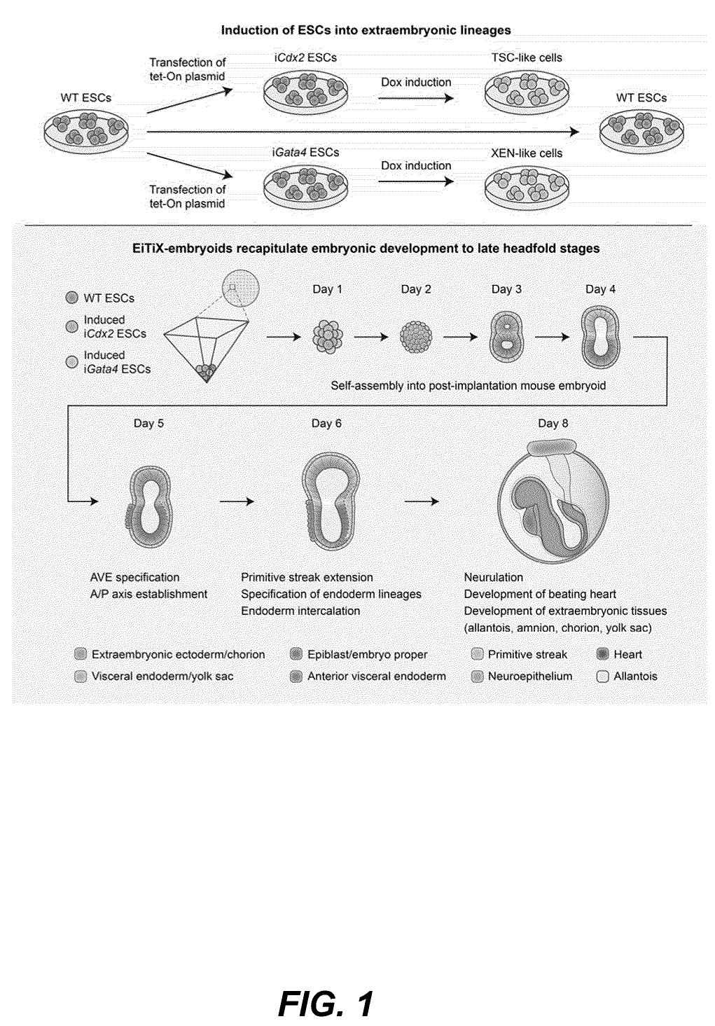

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

The term “about,” as used herein when referring to a measurable value such as an amount of a compound, dose, time, temperature, and the like, is meant to encompass variations of 10%, 5%, 1%, 0.5%, or even 0.1% of a specified amount.

The term “stem cell” as used herein can refer to a cell capable of retaining a constant potential for differentiation even after cell division. Examples of stem cells include: embryonic stem cells with pluripotency derived from a fertilized egg or clone embryo; epiblast stem cells; trophoblast stem cells; extraembryonic endoderm (XEN) stem cells; somatic stem cells and pluripotent stem cells that are present in tissues in a living organism e.g. hepatic stem cells, dermal stem cells, and reproductive stem cells that serve as the bases for respective tissues; pluripotent stem cells derived from reproductive stem cells; pluripotent stem cells obtained by nuclear reprogrammed somatic cells; totipotent stem cells and non-totipotent stem cells and the like.

The term “pluripotent stem cell” (PSC) as used herein refers to a stem cell permitting in vitro culture and having the potential for differentiating into all cells, but the placenta. The pluripotent stem cell has the potential to differentiate into any of the three germ layers: endoderm (which forms structures such as the gastrointestinal tract and the respiratory system), mesoderm (which forms structures such as the musculoskeletal system, the vascular system and the urogenital system), or ectoderm (which forms epidermal tissues and the nervous system).

The term “embryonic stem cell” (ES cell or ESC) as used herein refers to a pluripotent stem cell derived from the inner cell mass of a blastocyst, which is an early-stage preimplantation embryo. It is envisaged that such cells may express genes involved in the naive pluripotency network (Oct4/Nanog, Sox2, Klf4 etc). Such cells may also have Oct4 proximal enhancer activity. They may contribute to all embryonic tissues in chimeras. The ES cells may be derived from mammalian embryos, obtained from iPS cells or obtained from appropriate cell lines. Non-limiting examples of said stem cells include embryonic stem cells of a mammal or the like established by culturing a pre-implantation early embryo, embryonic stem cells established by culturing an early embryo prepared by nuclear-transplanting the nucleus of a somatic cell, induced pluripotent stem cells (iPS cells) established by transferring several different transcriptional factors to a somatic cell, and pluripotent stem cells prepared by modifying a gene on a chromosome of embryonic stem cells or iPS cells using a gene engineering technique. More specifically, embryonic stem cells include embryonic stem cells established from an inner cell mass that constitutes an early embryo, embryonic stem cells established from a primordial germ cell, cells isolated from a cell population possessing the pluripotency of pre-implantation early embryos (for example, primordial ectoderm), and cells obtained by culturing these cells.

The term “trophoblast stem cell” as used herein refers to stem cells derived from the trophoblast lineage of the embryo. The trophoblast stem cells are preferably not extra-embryonic cells derived from the two cell types which are precursors of the human placenta: the cytotrophoblast and the syncytiotrophoblast. These cells can be derived at late pre-implantation stages E4.5 or early post-implantation stages (E5.5) but the resulting cell lines are equivalent to the stem cell compartment existing in the extra-embryonic ectoderm of the post-implantation mouse egg cylinder. Transcription factors such as Elf5, Eomes, and Tfap2C mark this lineage. TS cells can also be considered as cells that are the precursors of the differentiated cells of the placenta. In the mouse, TS cells can be derived from outgrowths of either blastocyst polar trophectoderm or extraembryonic ectoderm, which originates from polar trophectoderm after implantation.

The term “extra-embryonic endoderm stem cell” (XEN stem cell) as used herein refers to stem cells derived from the extraembryonic endoderm of an embryo (e.g., a mouse embryo). The extraembryonic endoderm is typically a derivative of the hypoblast cells that migrate into the blastocyst cavity (beginning on day 8 of human embryonic development), and line the cavity, giving rise to the primary and definitive yolk sacs. The extraembryonic endoderm fills the remaining cavity of the blastocyst.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the phrase “culture medium” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

Mammalian blastocyst (e.g., mouse blastocyst) comprises three lineages at the time of implantation: the epiblast (EPI), the trophectoderm (TE), and the primitive endoderm (PE) that will give rise to the embryo proper, the placenta, and the yolk sac, respectively. By using stem cells derived from these lineages, researchers have developed several in vitro models to recapitulate various events of post-implantation development. One approach has been to take solely mouse embryonic stem cells (ESCs) and, by applying exogenous stimuli, induce them to establish anterior-posterior polarity and mimic basic body axis formation and aspects of gastrulation, somitogenesis, cardiogenesis, and neurulation. Such so-called “gastruloids” demonstrate the ability of ESCs to be directed into complex developmental programs. However, these systems fail to capture the entire complexity of signaling and morphological events along the complete body axes. This is largely because they fail to recapitulate the spatiotemporal interplay of signaling pathways between embryonic and extraembryonic tissues, which is crucial to pattern the post-implantation mouse embryo. Consequently, they do not represent complete embryonic structures and lack the overall morphological resemblance to natural post-implantation mouse embryos.

A second approach was adopted to fully model the post-implantation mammalian embryo (e.g., mouse embryo) by promoting assembly of mammalian ESCs (e.g., mouse ESCs) with either extraembryonic trophoblast stem cells (TSCs), to direct formation of a post-implantation egg cylinder showing appropriate posterior development. Alternatively, a mixture of TSCs and extraembryonic endoderm (XEN) stem cells was also adopted to generate “ETX” embryos that develop anterior and posterior identity and gastrulation movements. Subsequently, by replacing XEN cells with ESCs harboring inducible Gata4 expression (iGata4 ESCs), it was possible to generate XEN cells at an earlier stage of development that could contribute to iETX embryos that were fully able to complete gastrulation movements.

One remaining complication of the iETX embryo model was that TSCs and ESCs require different culture media, necessitating the use of undefined culture conditions and increasing the difficulty of developing embryoids in the laboratory.

To overcome this challenge, provided herein includes an in vitro model that is entirely ESC-based. The in vitro embryo model is generated with embryonic and extraembryonic lineages using exclusively embryonic stem cells and can undergo advanced development to late headfold stages.

Disclosed herein includes an in vitro method of generating a synthetic embryo from embryonic stem cells. In some embodiments, the method comprises co-culturing a wild-type mammalian embryonic stem cell (ESC), a mammalian ESC overexpressing CDX2 transcription factor, and a mammalian ESC overexpressing GATA transcription factor under a condition allowing the ESCs to self-assemble into a gastrulating embryo structure and culturing the gastrulating embryo structure in a post-implantation culture medium under a condition allowing the gastrulating embryo structure to develop into a synthetic embryo. Disclosed herein also includes a synthetic embryo structure obtained by the method disclosed herein.

Disclosed herein also includes an embryo culturing device. In some embodiments, the culturing device comprises a rotating incubator comprising a culture medium, a plurality of gas cylinders, a gas mixer configured to receive gases from the plurality of gas cylinders, and a gas and pressure controller.

Generating Synthetic Embryos In Vitro from Embryonic Stem Cells

Provided herein includes an embryonic stem cell (ESC)-based in vitro embryo model and related methods, culture media and devices for generating the same. The ESC-based in vitro embryo model is generated with embryonic and extra-embryonic lineages derived exclusively from mammalian embryonic stem cells. The ESC-based in vitro model reconstitutes the three fundamental cell lineages (e.g., a pluripotent ESC lineage and two extraembryonic lineages) of the post-implantation mammalian embryo through transcription-factor-mediated reprogramming. The extra-embryonic trophoblast stem cells (TSCs) and extra-embryonic endoderm (XEN) stem cells are generated by transcription factor overexpression with wild type embryonic stem cells. In some embodiments described herein, the TSCs are effectively substituted with genetically modified ESCs that can overexpress CDX2 transcription factor and the XEN stem cells are effectively substituted with genetically modified ESCs that can overexpress GATA transcription factor (e.g., GATA4). The reconstitution of the three principal lineages of peri-implantation development exclusively from ESCs ensures simplified, defined, and consistent culture conditions to recapitulate the interactions between embryonic and extraembryonic tissues that facilitate development through gastrulation to neurulation or alike stages. The ESC-based in vitro culturing method also circumvents the use of undefined media to culture conventional extraembryonic cell lines. The embryo-like structure formed solely by ESCs are hereby termed “EiTiX-embryoids.” Some of the methods and compositions disclosed herein are also disclosed in Lau et al. “Mouse embryo model derived exclusively from embryonic stem cells undergoes neurulation and heart development” Cell Stem Cell, Vol. 29, Issue 10 (2022), P1445-1458, the content of which is incorporated herein by reference in its entirety.

Provided herein include methods for generating synthetic embryos in vitro from pluripotent stem cells (e.g., ESCs). In some embodiments, the pluripotent stem cells are mammalian pluripotent stem cells. In some embodiments, the pluripotent stem cells are ESCs. The method can comprise co-culturing a wild-type mammalian embryonic stem cell (ESC), a mammalian ESC overexpressing CDX2 transcription factor, and a mammalian ESC overexpressing GATA transcription factor under a condition allowing the ESCs to self-assemble into a gastrulating embryo structure and culturing the gastrulating embryo structure in a post-implantation culture medium under a condition allowing the gastrulating embryo structure to develop into a synthetic embryo.

In some embodiments, the synthetic embryo described herein is generated exclusively from embryonic stem cells. The methods described herein for generating a synthetic embryo does not comprise culturing an extra-embryonic stem cell (e.g., an extra-embryonic trophoblast stem cell and extra-embryonic endoderm stem cell) in any stage of the culturing process. In some embodiments, The methods described herein for generating a synthetic embryo does not comprise culturing an extra-embryonic trophoblast stem cell and/or an extra-embryonic endoderm stem cell in any stage of the culturing process. Rather, the extra-embryonic stem cells (e.g., extra-embryonic trophoblast stem cells and extra-embryonic endoderm stem cells) are replaced with genetically modified embryonic stem cells. The genetically modified ESCs can comprise an ESC carrying a Cdx2 transgene capable of overexpressing CDX2 transcription factor upon induction and an ESC capable of overexpressing GATA transcription factor upon induction.

In some embodiments, the method comprises co-culturing a wild-type embryonic stem cell (ESC) and genetically modified ESCs, in which the genetically modified ESCs comprise an ESC overexpressing CDX2 transcription factor and an ESC overexpressing GATA transcription factor. In some embodiments, the GATA transcription factor is GATA4. In some embodiments, the ESC overexpressing CDX2 demonstrates an at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 220-fold, 240-fold, 260-fold, 270-fold, 280-fold, 300-fold or greater increase in Cdx2 mRNA expression than a wild type ESC. In some embodiments, the ESC overexpressing GATA (e.g., GATA4) demonstrates an at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 220-fold, 240-fold, 260-fold, 270-fold, 280-fold, 300-fold or greater increase in Gala mRNA expression than a wild type ESC.

In some embodiments, the method further comprises generating the ESC overexpressing GATA transcription factor and the ESC overexpressing CDX2 transcription factor by contacting an ESC carrying an inducible Gala gene (e.g., Gata4 gene) and an ESC carrying an inducible Cdx2 gene with an inducer (e.g., doxycycline). The contacting can be performed for any duration suitable to increase the mRNA expression to a desired level. For example, the contacting can be performed for a duration of about 2, 3, 4, 5, 6, 7, 8, 9, 10, 11, 12, 13, 14, 15, 16, 17, 18, 19, 20, 21, 22, 23, 24, or more hours. In some embodiments, the contacting duration is about 2-10 hours, optionally about 6 hours. In some embodiments, the induction (e.g., dox induction) is for a duration of about, at least, at least about, at most or at most about 1 day, 2 days, 3 days, 4 days, 5 days, 6 days, 7 days or longer. In some embodiments, the inducer is supplied during the co-culturing process. Accordingly, the method can comprise co-culturing a wild-type mammalian ESC, an ESC carrying an inducible Gala gene (e.g., Gata4 gene) and an ESC carrying an inducible Cdr2 gene in the presence of an inducer. The duration of the induction and/or the concentration of the inducer can be modulated, for example, by increasing or decreasing the concentration or amount of the inducer in the culture medium or by increasing or decreasing of the duration time of the inducer present in the culture medium. In some embodiments, the induction can be terminated by removing the inducer from the culture medium by, for example, replacing or replenishing the culture medium with fresh culture medium free of the inducer. Alternatively or in addition, modified mammalian ESCs can be induced prior to co-culturing with the wild-type ESC.

In some embodiments, the wild-type embryonic stem cell (ESC), the ESC overexpressing CDX2 transcription factor, and the ESC overexpressing GATA transcription factor are cultured in a pre-implantation and/or peri-implantation culture medium for up to 6 days (e.g., 1 day, 2 days, 3 days, 4 days, 5 days or 6 days).

The numbers or amounts of wild type ESCs and genetically modified ESCs can be at any suitable ratio which can vary in different embodiments. In some embodiments, the ESCs overexpressing CDX2 are provided at an amount in excess of the wild type ESCs and the ESCs overexpressing GATA. For example, the ESCs overexpressing CDX2 can be provided at an amount or a cell count at least 2, 3, 4, 5, 6, 7, or 8 times greater than the wild type ESCs and/or the ESCs overexpressing GATA. ESCs overexpressing GATA and wild type ESCs can be provided in the same or different amount. In some embodiments, the ESCs overexpressing GATA and wild type ESCs are provided in about the same amount (e.g., at a ratio of 1:1). In some embodiments, the ratio between the wild type ESC, the ESC overexpressing GATA (e.g., GATA4) and the ESC overexpressing CDX2 is from about 1:1:1 to about 1:1:10, such as 1:1:1, 1:1:2, 1:1:3, 1:1:4, 1:1:5, 1:1:6, 1:1:7, 1:1:8, 1:1:9, 1:1:10 or higher. In some embodiments, the ratio between the wild type ESC, the ESC overexpressing GATA (e.g., GATA4) and the ESC overexpressing CDX2 is from about 1:1:5 to about 1:1:8, such as 1:1:6 or 1:1:7.

The co-culturing allows the ESCs to self-assemble into a gastrulating embryo structure. In some embodiments, the gastrulating embryo structure resembles an E6.0-E7.5 natural embryo structure, optionally an E6.0-E6.5, E6.5-E7.0, or E7.0-E7.5 natural embryo structure. The gastrulating embryo structure can be further cultured in a post-implantation culture medium, e.g., for at least 3 days. Culturing the gastrulating embryo structure in the post-implantation culture medium allows the gastrulating embryo structure to develop into a synthetic embryo that resembles an E8.0-E8.5 natural embryo structure or beyond. In some embodiments, the mammalian ESCs can be mouse ESCs.

In some embodiments, the methods disclosed herein do not comprise any in vivo step. In some embodiments, none of the wild-type ESC, the ESC overexpressing CDX2 transcription factor, the ESC overexpressing GATA transcription factor, the gastrulating embryo structure, and the synthetic embryo is present in an in vivo environment in any of the culturing steps disclosed herein. The in vivo environment can comprise a tissue, an organ, an organism, or a combination thereof.

In some embodiments, the ESC-derived synthetic embryos obtained using the methods, culture media and devices disclosed herein can develop beyond gastrulation and neurulation to the equivalent of natural embryos 8.5 days post-fertilization or beyond. In some embodiments, the ESC derived synthetic embryos described herein can undergo development from pre-gastrulation stages through late gastrulation stages to neurulation stages (e.g., from embryonic day 5.5 to 8.5), developing anterior-posterior axis, headfolds, brain, a beating heart structure, and extraembryonic tissues including a yolk sac and chorion. Comparing single-cell RNA sequencing from individual structures with time-matched natural embryos identifies remarkably similar transcriptional programs across lineages, demonstrating an extraordinary plasticity of ESCs to self-organize and generate a whole-embryo-like structure.

### Stem Cells and Embryo Development

Disclosed herein are methods, compositions, culture media and devices for culturing mammalian pluripotent stem cells (e.g., embryonic stem cells) in vitro. In some embodiments, the mammalian pluripotent stem cells comprise an embryonic stem cell. In some embodiments, the method comprises co-culturing a wild-type mammalian embryonic stem cell (ESC), a mammalian ESC overexpressing CDX2 transcription factor, and a mammalian ESC overexpressing GATA transcription factor in a pre-implantation and peri-implantation culture medium under a condition allowing the ESCs to self-assemble into a post-implantation structure, such as a gastrulating embryo structure. The method can further comprise culturing the gastrulating embryo structure in a post-implantation culture medium under a condition allowing the gastrulating embryo structure to develop into a synthetic embryo In some embodiments, the mammalian embryonic stem cells can develop into a gastrulating embryo structure, a neurulating embryo structure and beyond under a suitable culturing condition in a suitable culture medium. A suitable culturing condition can comprise a static cell culture condition, a dynamic cell culture condition, or a combination thereof (e.g., a static condition followed by a dynamic condition).

While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. The embryo described herein is generated from culturing in vitro embryonic stem cells under appropriate conditions and resembles a natural embryo produced in vivo of a corresponding stage, such as having similar morphology, length, weight, cell type compositions and expression of developmental marker genes.

A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo. The earliest Carnegie stages are as follows in Table 1.

TABLE 1
Carnegie Stages of Development

| | Days since | |
|---|---|---|
| Carnegie | ovulation | |
| stage | (approx.) | Characteristic events/structures |
| 1 | 1 | fertilization; polar bodies |
| 2 | 2-3 | cleavage; morula; compaction |
| 3 | 4-5 | blastocyst and blastocoele; |
| | | trophoblast and embryoblast |
| 4 | 6 | syncytiotrophoblast; cytotrophoblast; |
| | | anchoring to endometrium |
| 5(a) | 7-8 | implantation; embryonic disc; bilaminar |
| | | germ disc; primary yolk sac; |
| 5(b) | 9-10 | formation of trophoblast lacunae; complete |
| | | penetration into endometrium; amniotic |
| | | cavity; primary umbilical vesicle |
| 5(c) | 11-16 | pre-chordal plate; extra-embryonic mesoblast; |
| | | secondary yolk sac |
| 6 | 17 | primitive streak, primitive node, primitive |
| | | groove; secondary umbilical vesicle; primordial |
| | | germ cells; body stalk; early gastrulation. |
| 7 | 19 | Gastrulation; neural plate; start of hematopoiesis. |
| 8 | 23 | Primitive pit |
| 9 | 25 | Neural groove; neural folds; septum transversum; |
| | | placode; early heart. |

In some embodiments, the mammalian embryos generated herein are mouse embryos. Theiler has established numbered stages of murine development. The earliest stages, as applied to (C57BLxCBA)F1 mice, arc described in the “emouse digital atlas” (www.emousealtas.org) as follows in Table 2.

TABLE 2
Theiler Stages of Development

Theiler

| Stage | Dpc* (range) | | Cell number | | (C57BL × CBA)Fl mice |
|---|---|---|---|---|---|
| 1 | 0-0.9 | (0-2.5) | 1 | | One-cell egg |
| 2 | 1 | (1-2.5) | 2-4 | | Dividing egg |
| 3 | 2 | (1-3.5) | 4-16 | (or 8-16) | Morula |
| 4 | 3 | (2-4) | 16-40 | (or 16-32) | Blastocyst, inner cell mass apparent |

| | | | |
|---|---|---|---|
| 5 | 4 | (3-5.5) | Blastocyst (zona-free) |
| 6 | 4.5 | (4-5.5) | Attachment of blastocyst; primary endoderm |
| | | | covers blastocoelic surface of inner cell mass |
| 7 | 5 | (4.5-6) | Implantation and formation of egg cylinder; |
| | | | Ectoplacental cone appears, enlarged epiblast, |
| | | | primary endoderm lines mural trophectoderm |
| 8 | 6 | (5-6.5) | Differentiation of egg cylinder. Implantation |
| | | | sites 2 × 3 mm. Ectoplacental cone region invaded |
| | | | by maternal blood, Reichert's membrane and |
| | | | proamniotic cavity form |
| 9a | 6.5 | (6.25-7.25) | Pre-streak(PS). advanced endometrial reaction, |
| | | | ecto lacental cone invaded by blood, |
| | | | extraembryonic ectoderm, embryonic axis visible |
| 9b | | | Early streak(ES), gastrulation starts, first |
| | | | evidence of mesoderm |
| 10a | 7 | (6.5-7.75) | Mid streak (MS), amniotic fold starts to form |
| 10b | | | Late streak, no bud (LSOB), exocoelom |
| 10c | | | Late streak, early bud (LSEB), allantoic bud |
| | | | first appears, node, amnion closing |
| 11a | 7.5 | (7.25-8) | Neural plate (NP), head process developing, |
| | | | amnion complete |
| 11b | | | Late neural plate (LNP), elongated allantoic |
| | | | bud |
| 11c | | | Early head fold (EHF) |
| 11d | | | Late head fold (LHF), foregut invagination |
| 12a | 8 | (7.5-8.75) | 1-4 somites, allantois extends, first branchial |
| | | | arch, heart starts to form, foregut pocket visible, |
| | | | preotic sulcus at 2-3 somite stage) |
| 12b | | | 5-7 somites, allantois contacts chorion at the end |
| | | | of TS12, Absent 2^{nd} arch, >7 somites |
| 13 | 8.5 | (8-9.25) | Turning of the embryo, 1^{st} branchial arch has |
| | | | maxillary and mandibular components, 2^{nd} arch |
| | | | present; Absent 3rd branchial arch |
| 14 | 9 | (8.5-9.75) | Formation & closure of ant. neuropore, otic |
| | | | pit indented but not closed, 3^{rd} branchial arch |
| | | | visible; Absent forelimb bud |
| 15 | 9.5 | (9-10.5) | Formation of post. neuropore, |
| | | | forelimb bud, forebrain vesicle subdivides; |
| | | | Absent hindlimb bud, Rathke's pouch |
| 16 | 10 | (9.5-10.75) | Posterior neuropore closes, Formation of |
| | | | hindlimb & tail buds, lens plate, Rathke's pouch; |
| | | | the indented nasal processes start |
| | | | to form; Absent thin & long tail |
| 17 | 10.5 | (10-11.25) | Deep lens indentation, adv. devel. of brain tube, |
| | | | tail elongates and thins, umbilical hernia starts to |
| | | | form; Absent nasal pits |
| 18 | 11 | (10.5-11.25) | Closure of lens vesicle, nasal pits, cervical |
| | | | somites no longer visible; |
| | | | Absent auditory hillocks, anterior footplate |
| 19 | 11.5 | (11-12.25) | Lens vesicle completely separated from the |
| | | | surface epithelium, Anterior, but no posterior, |
| | | | footplate. Auditory hillocks first visible; Absent |
| | | | retinal pigmentation and sign of fingers |
| 20 | 12 | (11.5-13) | Earliest sign of fingers, (splayedout), posterior |
| | | | footplate apparent, retina pigmentation apparent, |
| | | | tongue well-defined, brain vesicles clear; |
| | | | Absent 5 rows of whiskers, indented |
| 21 | 13 | (12.5-14) | Anterior footplate indented, elbow and wrist |
| | | | identifiable, 5 rows of whiskers, umbilical hernia |
| | | | now clearly apparent; |
| | | | Absent hair follicles, fingers separate distally |
| 22 | 14 | (13.5-15) | Fingers separate distally, only indentations |
| | | | between digits of the posterior footplate, long |
| | | | bones of limbs present, hair follicles in pectoral, |
| | | | pelvic and trunk regions; |
| | | | Absent open eyelids, hair follicles in cephalic |
| | | | region |

| 23 | 15 | Fingers & Toes separate, hair follicles also in |
|---|---|---|
| | | cephalic region but not at periphery of vibrissae, |
| | | eyelids open; |
| | | Absent nail primordia, fingers 2-5 parallel |
| 24 | 16 | Reposition of umbilical hernia, eyelids closing, |
| | | fingers 2-5 are parallel, nail primordia visible on |
| | | Toes; |
| | | Absent wrinkled skin, fingers & toes joined |
| | | together |
| 25 | 17 | Skin is wrinkled, eyelids are closed, umbilical |
| | | hernia is gone; |
| | | Absent ear extending over auditory meatus, long |
| | | whiskers |
| 26 | 18 | Long whiskers, eyes barely visible through |
| | | closed eyelids, ear covers auditory meatus |
| 27 | 19 | Newborn Mouse |

*“dpc” indicates days post conception, with the morning after the vaginal plug is found being designated 0.5 dpc or E0.5.

In some embodiments, the developmental stage of a synthetic embryo generated herein can be defined according to its embryonic day. As used herein, the term “embryonic day (E)” in the context of a mammalian embryo (e.g., mouse embryo) refers to an embryo having developmental characteristic of an in vivo (in-uterine tube or in utero) mammalian embryo counterpart at the specified day following fertilization, wherein E0 is considered as the fertilized egg.

In some embodiments, the methods and compositions described herein can enable culture up to or through to post-implantation stages corresponding to Theiler stage 7, 8, 9(a), 9(b), 10(a), 10(b), 10(c), 11(a), 11(b), 11(c), 11(d), 12(a), 12(b), 13, 14, 15, 16 and beyond, Carnegei stage (a), 5(b), 5(c), 6, 7, 8, 9 and beyond, and corresponding stages in other species. In some embodiments, the synthetic embryo generated herein can reach post-implantation stages of E4, E4.5, E5, E5.5, E6, E6.5, E7, E7.5, E8, E8.5, E9, E9.5 and beyond.

The methods and compositions herein described can be applied to embryos from any suitable mammalian species, such as: primates, including humans, great apes (e.g. gorillas, chimpanzees, orangutans), old world monkeys, new world monkeys; rodents (e.g. mice, rats, guinea, pigs, hamsters); cats; dogs; lagomorphs (including rabbits); cows; sheep; goats; horses; pigs; and any other livestock, agricultural, laboratory or domestic mammals.

The methods and compositions herein described can be applied to an embryo from a non-human mammal, including but not limited to those described above. Thus, any of the culture media embodiments defined herein may be capable of supporting development of a non-human mammalian embryo on a substrate from a pre-implantation stage of development to a post-implantation stage of development.

The term “pre-implantation stage” can be used herein to refer to a stage of development earlier than the stage corresponding to Theiler stage 7, Carnegie stage 5(a), and corresponding stages in other species. As used herein, the term “post-implantation stage” can refer to a stage of development later than the stage corresponding to Theiler stage 7, Carnegie stage 5(a), and corresponding stages in other species. A “post-implantation stage” may be determined by detecting the up-regulation of one or more genes by the embryo. For example, such a stage may be determined by detecting one or more of the following changes: the epiblast up-regulates Fgf5; the primitive endoderm differentiates into visceral endoderm that up-regulates Cer1 in a subpopulation of cells (the anterior visceral endoderm); the visceral endoderm up-regulates Eomes; and the trophectoderm up-regulate Hand1.

Stem cells (e.g., mammalian pluripotent stem cells such as embryonic stem cells) can be cultured using the media, kits and methods described herein. In the embodiments described herein, the stem cells comprise pluripotent stem cells (PSCs). A PSC can be obtained from a fertilized egg, clone embryo, reproductive stem cell, or stem cell in tissue. Also included are cells having differentiation pluripotency similar to that of embryonic stem cells, conferred artificially by transferring several different genes to a somatic cell (also referred to as induced pluripotent stem cells or iPS cells). Induced pluripotent stem cells may be derived from any suitable source (e.g. hair follicles, skin cells, fibroblasts, etc.). Pluripotent stem cells can be prepared by known methods in the art. Any of the stem cells as defined herein may be derived from diseased or non-diseased tissue. Stem cells can be from any suitable mammalian species, such as: primates, including humans, great apes (e.g. gorillas, chimpanzees, orangutans), old world monkeys, new world monkeys; rodents (e.g. mice, rats, guinea pigs, hamsters); cats; dogs; lagomorphs (including rabbits); cows; sheep; goats; horses; pigs; and any other livestock, agricultural, laboratory or domestic mammals. The presently disclosed methods may be applied to stem cells from any non-human mammal, including but not limited to those described above. In some embodiments, the non-human mammals are rodents. In some embodiments, the PSC cells disclosed herein are mammalian embryonic stem cells (ESCs).

As would be understood by a person of skill in the art, ESCs may be obtained from stem cell banks such as the UK stem cell bank from which one can acquire human stem cell lines for research. The Jackson Laboratory, US (who provide Jax mice) also stores and derives mouse ES cells which are available for purchase. It is preferred that the ESCs are obtained or are obtainable by a method that does not involve the destruction of human or non-human animal embryos.

### Synthetic Embryos at Different Developmental Stages

Provided herein include methods, compositions, and culture media for modeling mammalian embryo development by culturing pluripotent stem cells (e.g., embryonic stem cells). The methods, compositions and culture media disclosed herein can generate synthetic embryos through various developmental stages.

In some embodiments, the synthetic embryos generated using the methods and compositions described herein can reach a post-implantation (e.g., a post-implantation gastrulating stage or neurulating stage). In some embodiments, the synthetic embryos generated herein can reach an early gastrulation stage. In some embodiments, the synthetic embryos generated herein can reach a late gastrulation stage. In some embodiments, the synthetic embryos generated herein can reach an early neurulation stage. In some embodiments, the synthetic embryos generated herein can reach a late neurulation stage.

In some embodiments, the embryo structures generated using the methods and culture media described herein comprise post-implantation embryos, e.g., post-implantation pre-gastrulation embryo structure. As used herein, the term “post implantation pre gastrulation” in the context of a mammalian embryo (e.g., a mouse embryo) refers to an embryo following the implanting blastocyst stage and prior to the early gastrulation stage and is characterized by an egg cylinder-shape prior to symmetry breaking. An embryo of a post implantation pre gastrulation stage can be defined as Theiler stages TS7-TS8 (see Table 2). In some embodiments, the post-implantation, pre-gastrulation stage refers to E4.5-6.5, optionally, E4.5-6, optionally E5-6.5, optionally E5-5.5. In some embodiments, the post-implantation, pre-gastrulation stage refers to E5.5.

Embryonic stage of a synthetic embryo structure generated using the methods and culture media disclosed herein can be assessed by comparing to an in vivo natural embryo counterpart at the same developmental stage by multiple ways including, but not limited to, morphology, length, weight, cell type compositions and expression of developmental marker genes (e.g., Oct4, Nanog, Sox2, Klf4, Cdx2, Gata4, Gata6, Brachyury, Otx2, Fgf5 and others described in the Examples and known in the art) using specific antibodies or primers, or transcriptional profiling, single-cell RNA sequencing and other methods as further described in the Examples section. In some embodiments, the post-implantation pre-gastrulation embryo structure resembles an E5.0-E5.5 natural embryo structure. In some embodiments, the post-implantation pre-gastrulation embryo structure and an E5.0-E5.5 natural embryo structure have similar morphology, cell type compositions, and gene expression features.

In some embodiments, the embryo structures generated using the methods and culture media described herein comprise post-implantation gastrulating embryos, As used herein, the term “gastrulation” in the context of an embryo refers to an embryo following the expanded blastocyst stage and prior to the somitogenesis stage and is characterized by the formation of the primitive streak and epithelial to mesenchymal transition forming three germinal layers. The gastrulation process is generally considered as the process through which the bilaminar embryonic disc is changed into a trilaminar disc as an intraembryonic mesoderm appears between the ectoderm and endoderm. A gastrulation stage can be an early gastrulation stage, mid-gastrulation stage, or late or advanced gastrulation stage. An early gastrulation embryo in the context of a mammalian embryo (e.g., mouse embryo) can refer to an embryo following the post-implantation pre-gastrulation stage and prior to an advanced gastrulation stage and is characterized by egg cylinder shape with the primitive streak at the posterior side. An embryo of an early gastrulation stage can be defined as Theiler stages TS8-TS10 (see Table 2). In some embodiments, an early gastrulation stage refers to E5.0-E6.5. In some embodiments, a mid-gastrulation stage refers to E6.5-E7.

In some embodiments, culturing of a post-implantation pre-gastrulation embryo structure to its early gastrulation stage is continued allowing the post-implantation embryo structure to develop into a late gastrulation stage or to complete gastrulation. As used herein, the term “late gastrulation stage” in the context of a mammalian embryo (e.g., a mouse embryo) refers to an embryo following the early gastrulation stage and prior to the early somite stage and is characterized by an egg cylinder-shaped embryo with differentiated definitive endoderm, mesoderm and ectoderm layers. An embryo of a late gastrulation stage can be defined as Theiler stages TS10-TS11 (see Table 2). In some embodiments, a late gastrulation stage can correspond to E6.5-8, optionally E6.5-7.75, optionally E7.25-8, optionally E7-8. In some embodiments, a late or advanced gastrulation stage refers to about E7.0-E7.5.

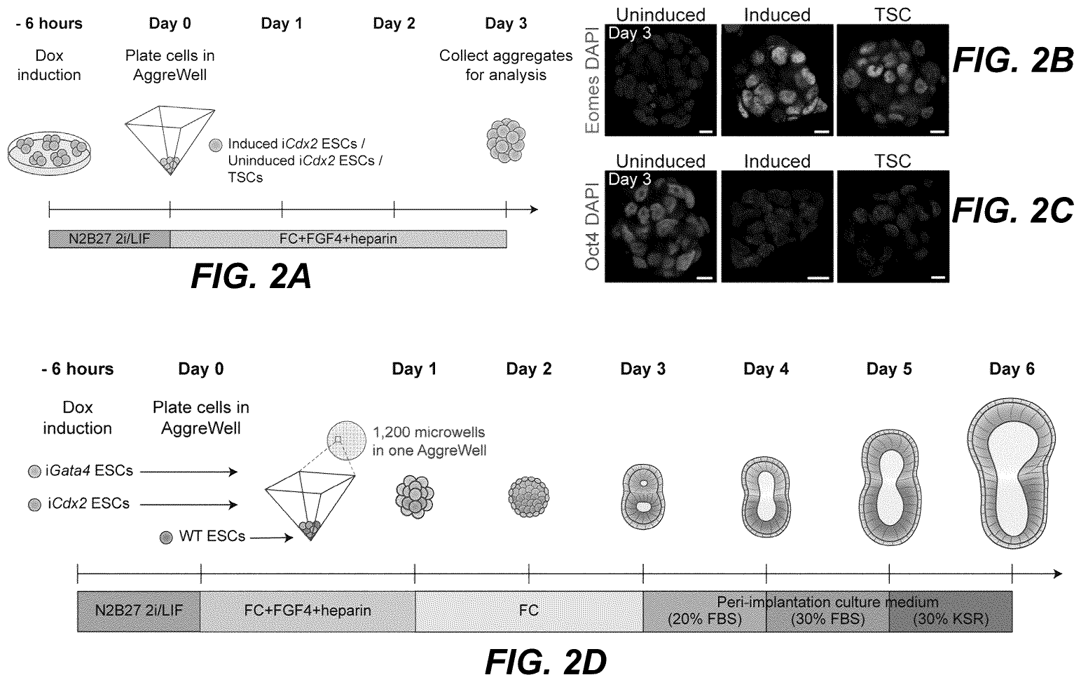

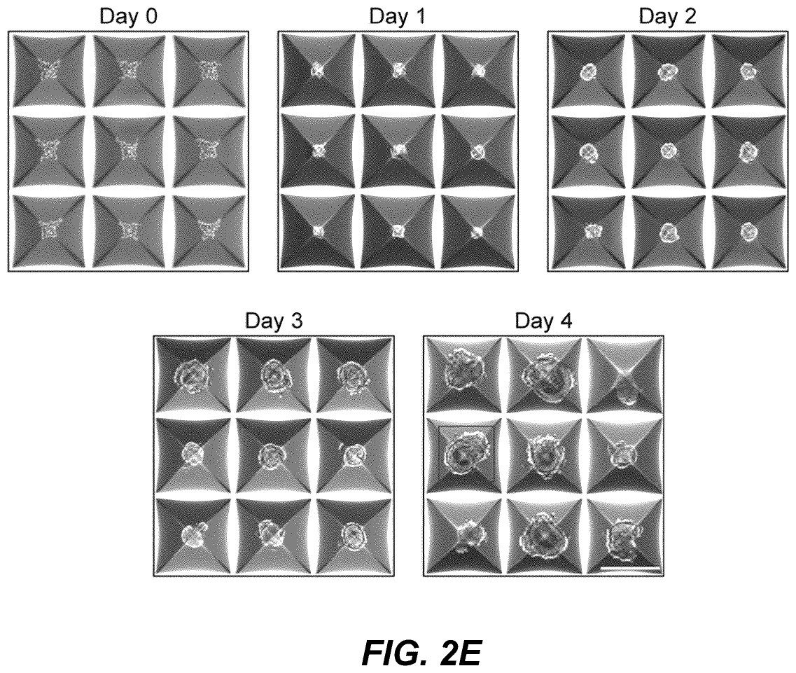

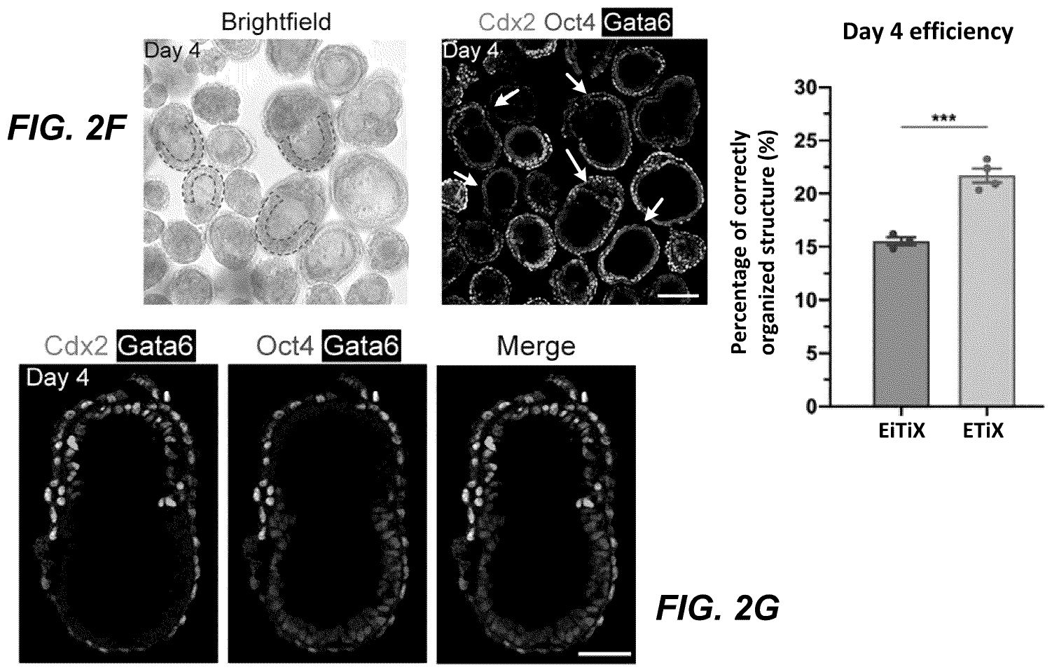

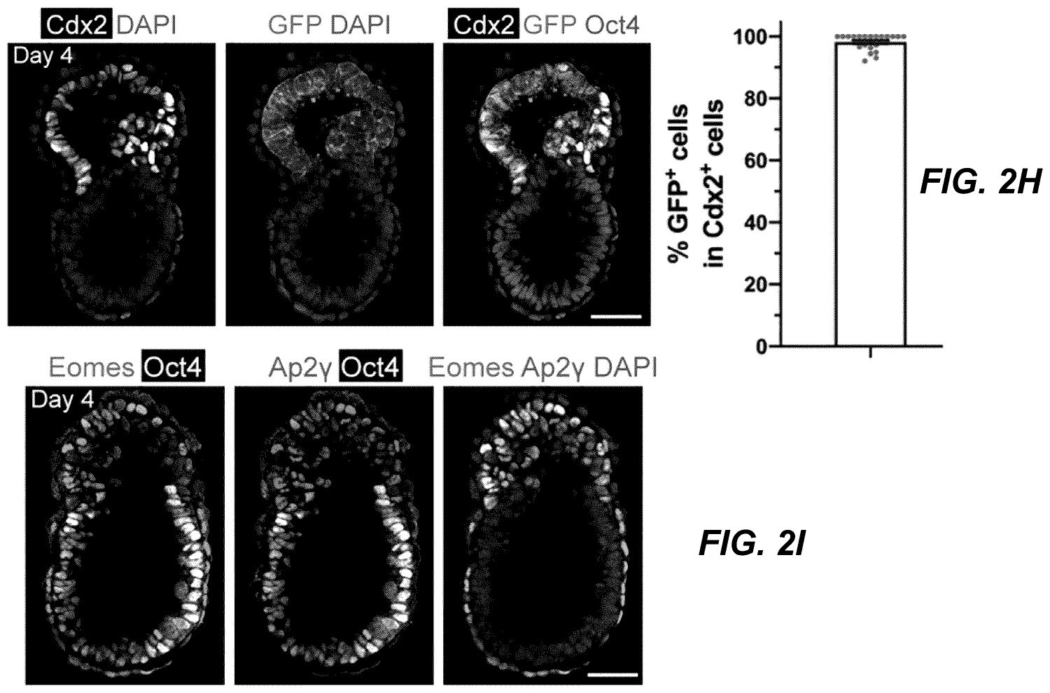

In some embodiments, the gastrulating embryo structures generated using the methods and culture media described herein resemble natural gastrulating embryos. In some embodiments, the synthetic gastrulating embryos and natural gastrulating embryos have similar morphology, cell type compositions, and gene expression features. In some embodiments, a synthetic embryo generated using the methods described herein resembles a post-implantation embryo structure (e.g., an early gastrulation stage) with established AVE and primitive streak, recapitulating anterior-posterior patterning as in natural post-implantation embryos (see, for example, FIGS. 2A-I). In some embodiments, a synthetic embryo generated using the methods described herein resembles a mid to late-gastrulation stage embryo structure. In some embodiments, co-culturing the ESCs as described herein in culture media such as in a pre-implantation culture medium (e.g., FC medium) and/or in a peri-implantation culture medium (e.g., IVC medium) can generate a post-implantation gastrulating embryo resembling an E6.0-E7.5 natural embryo structure (e.g., E6.0-E6.5, E6.5-E7.0, or E7.0-E7.5).

In some embodiments, culturing of a post-implantation embryo structure is continued allowing the post-implantation embryo structure to develop through and beyond gastrulation to neurulation. Neurulation is typically considered as an embryo developmental process which begins when the notochord induces the formation of the central nervous system by signaling the ectoderm germ layer above it to form the thick and flat neural plate. The neural plate folds in upon itself to form the neural tube that will later differentiate into the brain and the spinal cord of the central nervous system. A neurulating embryo generated herein can be an embryo resembling any of a series of morphological phases during the neurulation process. A neurulating embryo generated herein can be at the early neurulation stage, in the middle neurulation stage, or at the late neurulation stage.

In some embodiments, the neurulating embryo structure is generated by culturing a post-implantation embryo structure (e.g., a post-implantation gastrulating embryo structure) in a post-implantation culture media under a condition (e.g., a static condition and/or dynamic condition) allowing the post-implantation embryo structure to develop into a neurulating embryo structure and beyond. In some embodiments, culturing of the post-implantation gastrulating embryo structure can be for at least 3 days.

During culturing in the post-implantation culture medium, the post-implantation embryo can develop headfolds, a beating heart, allantois, and chorion structures. The post-implantation embryo can develop amnion-like and yolk-sac-like membranes that properly envelop the embryonic structures. Regions resembling blood islands, both in spatial localization and stereotypical red pigmentations, can be observed on the yolk-sac-like membrane. In some embodiments, the efficiencies of the synthetic embryos progressing from post-implantation pre-gastrulation stage to post-implantation late gastrulation stage to neurulation stage (e.g., from Day 5 to 6, Day 6 to 7, and Day 7 to 8) are between about 65% to 75%. In some embodiments, the synthetic embryo generated in the post-implantation culture medium have well defined structures resembling headfolds, heart, and tail.

In some embodiments, the neurulating embryo structures generated using the methods and culture media described herein resemble natural neurulating embryos. For example, the synthetic neurulating embryos and natural neurulating embryos can have similar morphology, cell type compositions, and gene expression features. In some embodiments, the synthetic embryo structure generated using the methods and culture media described herein resembles an E7-9 natural embryo structure, optionally an E7.5-8.5 natural embryo structure, optionally an E7.5-8.0 natural embryo structure, optionally an E8.0-8.5 natural embryo structure.

In some embodiments, the synthetic embryo structure generated herein can express neuroepithelium markers such as Sox1, Sox2, heart markers such as Myh2 and Gata4, chorion markers such as Hand1 and Keratin18, mesoderm marker, hematopoietic maker such as Runx1, and other markers such as OTX2, Foxa2, Sox17, Brachyury, Isl1 and others identifiable by a skilled person. In some embodiments, the heart markers Myh2 and Gata4 are expressed below headfolds, and a ventral view of the Gata4-expressing heart region revealed a morphology that resembles a linear heart tube. In some embodiments, the anterior region of the headfolds show robust Otx2 expression, indicating development of a forebrain. Robust expression of Sox2 and Brachyury at the region resembling a tail can be observed in some embodiments, which identifies a neuromesodermal progenitor population.

In some embodiments, the neurulating embryo structure can further develop into an embryo of somite stage or at a somitogenesis (e.g., from a late gastrulation and/or a neurulating stage to an early somite stage). As used herein, the term “somitogenesis” in the context of a mammalian embryo (e.g., mouse embryo) refers to an embryo following late gastrulation stage and prior to the early organogenesis stage and is characterized by the appearance of the first one to five somites distinguishable by bright field microscopy. An embiyo of an early somite stage can be defined as Theiler stages TS12-TS13 (see Table 2). In some embodiments, early somitogenesis refers to E7.5-9.25, optionally E7.5-8.75, optionally E8-9.25, optionally E8-9. In some embodiments, early somite stage refers to E8.5. In some embodiments, culturing a post-implantation gastrulating embryo structure in a post-implantation culture medium allows the gastrulating embryo structure to develop into a synthetic embryo at early somite stage.

In some embodiments, the synthetic embryo structure described herein comprise cells expressing markers such as NKX2-5, GATA4, and/or MYH2, and/or having gene expression signatures leading to somite formation.

In some embodiments, culturing a post-implantation gastrulating embryo structure in a post-implantation culture medium can allow the gastrulating embryo structure to develop into a synthetic embryo at an organogenesis stage and beyond. In some embodiments, the organogenesis stage is an early organogenesis stage. The term “early organogenesis” in the context of a mammalian embryo (e.g., mouse embryo) refers to an embryo following the somitogenesis stage and prior to the appearance of the heart beat stage and is characterized by formation of the neural tube and mesoderm migration. In some embodiments, the early organogenesis stage refers to E8-9, optionally E8-8.5, optionally E8.5 of a natural embryo. A synthetic embryo at the early organogenesis stage can demonstrate development of definitive endoderm, which gives rise to the gut and associated organs. The presence of foregut and hindgut pockets can be observed in a synthetic embryo at the early organogenesis stage. In some embodiments, the embryoid development described herein can proceed further beyond the early organogenesis stage in culture.

In some embodiments, the synthetic embryo generated herein can have established neuroepithelium, brain regions, a beating heart, and mesodermal populations, as well as developed extraembryonic tissues including yolk sac, chorion and amnion.

Culturing an embryo cell in vitro from stem cells (e.g., a mammalian pluripotent stem cell such as ESCs) can be effected until reaching early organogenesis or any developmental stage therein-between. In some embodiments, culturing a synthetic embryo in vitro can be continued allowing the synthetic embryo at the early organogenesis to develop onwards.

In some embodiments, the synthetic embryos generated using the methods and culture conditions described herein are mammalian embryos. In some embodiments, the mammalian embryos are non-human embryos, such as mouse embryos or rabbit embryos.

Culturing conditions mentioned above for generating synthetic embryos at different developmental stages, including media, type, pressure, oxygen concentrations and the like, are described in the sections below as well as in specific embodiments of mouse embryos in the Examples section.

Embryonic stages of the synthetic embryos described herein can be assessed compared to an in vivo or natural embryo counterpart at the same developmental stage by multiple ways including, but not limited to, morphology, length, weight, weight, expression of developmental marker genes using specific antibodies or primers, transcriptional profiling and the like, as further described hereinbelow and in the Examples section.

Morphology assessment of embryonic development can be performed by previously established morphological features such as described in Carnegie stages of development (see, for example, Table 1; Developmental stages in human embryos. R. O'Rahilly and F. Müller (eds), Carnegie Institution of Washington, Washington, D C, 1987), in Theiler stages of development (see, for example, Table 2; www.emouseatlas.org) or according to embryonic days.

In some embodiments, one or more developmental markers as described herein can be used to assess the developmental stage of a synthetic embryo structure. Numerous methods exist in the art for detecting the presence, absence, or amount of a marker gene product (e.g., mRNA and/or protein), as well as its localization in an embryo structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

In some embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also be possible to obtain cells from, e.g., the synthetic embryo cells and culture the cells in vitro, such as to obtain a larger population of cells from which RNA can be extracted. Methods for establishing cultures of non-transformed cells, i.e., primary cell cultures, are known in the art. In some embodiments, cells can be dissociated (e.g., by enzymatic or mechanical means), and isolated by methods known in the art (e.g., Fluorescence-Activated Cell Sorting, Microfluidics, etc.)

When isolating RNA from, e.g., synthetic embryo structures at various developmental stages and/or cells comprising said synthetic embryo structures, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used. Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 66,186,796; 6,379,897; 6,664,377; 6,451,536; 548,257; U.S. 20030157485). Serial Analysis of Gene Expression (SAGE) can also be performed (See for example U.S. Patent Application 20030215858). In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. Methods for constructing sequencing libraries are known in the art.

In some aspects of the disclosure the single cell sequencing is high-throughput single cell RNA sequencing. In certain embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In certain embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In certain embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

The activity or level of a lineage marker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-marker protein antibodies, such as intrabodies, may also be used for imaging purposes, for example, to detect the presence of marker protein in cells or, e.g., an embryo. Suitable labels include radioisotopes, iodine (¹²⁵I, ¹²¹I), carbon (⁴C), sulphur (³⁵S), tritium (³H), indium (¹¹²In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or may be prepared according to methods known in the art. Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

### Substrates for Culturing Synthetic Embryos

In some embodiments, wild-type and genetically modified ESCs, post-implantation embryos, gastrulating embryos, neurulating embryos and/or other synthetic embryos described herein are cultured in a substrate. In some embodiments, the method comprises transferring an embryo from one substrate to another substrate. The substrates used in the methods disclosed herein can be the same or different. For example, the mammalian pluripotent stem cells (e.g., ESCs) can be cultured in a first substrate to form an embryo structure of a first developmental stage (e.g., a post-implantation gastrulating embryo structure). The post-implantation gastrulating embryo can be transferred to a second substrate to develop into an embryo structure of a second developmental stage (e.g., a neurulating embryo). The neurulating embryo can be transferred to a third substrate to develop into a synthetic embryo of another developmental stage. The first substrate, second substrate and third substrate can be a same type or different types. In some embodiments, the first substrate, second substrate and third substrate are of different types. For example, the first substrate and the second substrate can be a microwell plate comprising inverted pyramidal microwells and the third substrate can be a rotating bottle culture unit such as a rotating incubator. An exemplary rotating incubator is the BTC rotating bottle culture unit by B.T.C. Engineering, Cullum Starr Precision Engineering Ltd.

The substrate as used herein can comprise a dish, a U-plate, a flask, or a microwell plate. The microwell plate can comprise inverted pyramidal microwells. The size (e.g., depth and/or diameter) of each of the inverted microwells can vary. Each of the inverted-pyramidal microwells can be about 400 μm or about 800 μm in size. Each of the inverted-pyramidal microwells can be about 400 μm or about 800 μm in diameter. In some embodiments, each of the inverted pyramidal microwells can be about 100, 200, 300, 400, 500, 600, 700, 800, 900 μm, 1 mM in size and/or diameter, or a number or a range between any two of these values. Each microwell (e.g., receptacle) may have a depth of about 250 μm to about 400 μm, e.g. about 300 μm to about 350 μm. Additionally or alternatively, said plurality of receptacles may have a mean depth of about 250 μm to about 400 μm, e.g. about 300 μm to about 350 μm. Especially when the receptacles are wells, they may be ordered on the substrate in an array, i.e. in a grid pattern having regular spacing in substantially orthogonal directions. Whatever the topography of the substrate, the substrate may carry one or more embryos. Where the substrate comprises one or more receptacles, each said receptacle may independently contain one or more embryos, e.g. 2, 3, 4 or 5 embryos, or more. In some embodiments, each embryo structure is located in a different respective well. In alternative embodiments, each receptacle comprises a plurality embryos, e.g. 2, 3, 4 or 5 embryos, or more.

The methods disclosed herein may be applied in culture volumes of any appropriate size. For example, the culture volume per embryo may be about 50 μl to about 10 ml, optionally about 100 μl to about 5 ml, optionally about 250 μl to about 5 ml, optionally about 1 ml to about 5 ml. The culture volume per embryo may be about 100, 150, 200, 250, 300, 350, 400, 450, 500, 550, 600, 650, 700, 750, 800, 850, 900, 950, 1000, 1500, 2000 μl or more.

### Conditions for Culturing Synthetic Embryos

The mammalian pluripotent stem cells (e.g., ESCs) and/or embryo-like structures at various developmental stages can be cultured in different culturing conditions. Culturing conditions can include type, media, pressure, oxygen concentration and the like. A culturing condition can be a static condition or a dynamic condition. In some embodiments, the pluripotent stem cells (e.g., ESCs) and/or embryos herein described are cultured in a static condition. For example, co-culturing a wild-type mammalian ESC, a mammalian ESC overexpressing CDX2 transcription factor, and a mammalian ESC overexpressing GATA transcription factor can be performed under a static condition. In some embodiments, culturing a gastrulating embryo structure in a post-implantation culture medium is performed under a static condition.

As used herein, the term “static condition” refers to a cell culture condition that is carried out without agitation of the culture. For example, the mammalian ESCs can be cultured under a first static condition in a static incubator to form a post-implantation embryo (e.g., a post-implantation gastrulating embryo) for a certain time period (e.g., 1, 2, 3, 4, 5, 6 or more days). The post-implantation gastrulating embryo can be cultured under a second static condition in a static incubator to develop into a neurulating embryo for a certain time period (e.g., 1, 2, 3 or more days). The first static condition and the second static condition can be the same or different. For example, different culture media may be used for the two (or more) static conditions. The ESCs and embryos can be cultured under static conditions for 1, 2, 3, 4, 5, 6, 7 or more days. In some embodiments, the stem cells and/or embryos are cultured under a static condition for up to 7 days. In some embodiments, the stem cells and/or embryos are cultured under a static condition up to E8.0.

In some embodiments, the methods comprise culturing in a static condition followed by a dynamic condition. Accordingly, the methods disclosed herein comprises culturing the embryos (e.g., neurulating embryos) under a dynamic condition in a culture chamber. For example, culturing a gastrulating embryo structure in a post-implantation culture medium can comprise culturing the gastrulating embryo structure under a static condition (e.g., for about 2 days) followed by culturing under a dynamic condition in a culture chamber (e.g., for at least 1 day). As used herein, the term “dynamic condition” refers to a cell culture condition that is carried out with agitation (e.g., suspension agitation), including but not limited to rotating, rolling, shaking, inverting, of the culture. Non-limiting examples of dynamic cultures or dynamic culture conditions include a roller culture (a culture on a rolling device), a shaker culture (a culture on a shaker, e.g. orbital shaker), or other dynamic cultures identifiable to a person skilled in the art.

In some embodiments, the dynamic condition comprises providing a plurality of gases to the culture chamber. The plurality of gases can comprise O₂, N₂, CO₂, H₂, H₂O, or a combination thereof. In some embodiments, the plurality of gases comprises O₂, N₂, and CO₂. In some embodiments, a mixture of gases is provided into a rotating culture chamber containing one or more embryos. In some embodiments, the dynamic condition comprises a gas pressure of about 0.5 to about 3 pounds per square inch (psi) in the culture chamber. In some embodiments, the gas pressure in the culture chamber is less than the sea level atmospheric pressure. For example, the gas pressure in the culture chamber is about 0.5 to less than 1 psi. In some embodiments, the gas mixture is delivered to the culture chamber at about 0.5 psi.

In some embodiments, the dynamic condition comprises supplying oxygen at a constant concentration to the culture chamber. For example, oxygen can be supplied to the culture chamber and maintained at a constant concentration of about 10%, 11%, 12%, 13%, 14%, 15%, 16%, 17%, 18%, 19%, 20%, 21%, 22%, 23%, 24%, 25% or higher. In some embodiments, oxygen is maintained in a constant concentration of about 21%.

In some embodiments, the oxygen is supplied to the culture chamber at an increasing concentration. The oxygen concentrations can be increased throughout the culturing starting from 5% up to 6%, 7%, 8%, 9%, 10%, 11%, 12%, 13%, 14%, 15%, 16%, 17%, 18%, 19%, 20%, 21%, 22%, 23%, 24%, 25% or higher at any suitable time interval (e.g., daily intervals). In some embodiments, the oxygen concentration is incrementally increased from about 5% to about 13% to about 18% to about 21% at daily intervals. In some embodiments, the culture chamber can have an atmosphere comprising an increasing oxygen concentration from about 5% to about 25% oxygen level, optionally from about 5% to about 13%, optionally from 13% to about 18%, optionally from about 18% to about 21%. In some embodiments, the increasing is effected by 1.5-2.5 fold or 1.5-2 fold in every step of the increasing. In some embodiments, the oxygen level in the culture chamber is less than 30%. In some embodiments, the dynamic condition used in culturing the neurulating embryos does not comprise decreasing the oxygen concentration throughout the culturing. In some embodiments, the increasing of oxygen concentration is effected every 0.5-2 days, every 0.5-1.5, every 1-2, or every 1-1.5 days of the culturing. In some embodiments, the increasing is effected every 20-28 hours of the culturing. In some embodiments, the increasing is effected at daily intervals.

In some embodiments, the dynamic condition comprises supplying oxygen and carbon dioxide at a constant concentration to the culture chamber. For example, carbon dioxide can be supplied to the culture chamber and maintained at a constant concentration of about 1%, 2%, 3%, 4%, 5%, 6%, 7% or 8%. In some embodiments, carbon dioxide is maintained in a constant concentration of about 5%. In some embodiments, embryos are cultured in a roller culture and maintained in a constant atmosphere of 21% oxygen and 5% carbon dioxide.

In some embodiments, for cultures starting at post-implantation or later stages (e.g., a neurulating state), the embryos are incubated on rotating bottles culture unit inside an incubator system, such as the BTC rotating bottle culture unit by B.T.C. Engineering, Cullum Starr Precision Engineering Ltd, during all the time of culture. The neurulating embryo can be cultured under a dynamic condition described herein for at least one day (e.g., 1, 2, 3, 4 or more days). In some embodiments, the embryos (e.g., neurulating embryos) are cultured under a dynamic condition from E7.5-E8.0 to at least E8.5 (e.g., E8.5, E9, or higher). In some embodiments, the embryo (e.g., neurulating embryos) can be cultured under a dynamic condition in a culture chamber for at least one day to develop into a synthetic embryo forming headfolds, brain and a beating heart structure and developing extraembryonic tissues including yolk sac and chorion.

### Culture Media

In some embodiments, the method comprises co-culturing mammalian pluripotent stem cells (e.g., ESCs comprising wild-type and genetically modified ESCs) in a feeder cell (FC) media, optionally passaging the ESCs in the feeder cell media at least two times (e.g., 2, 3, 4 or more times). The mammalian pluripotent stem cells can be cultured in the FC media for 1, 2, 3, or 4 days. In some embodiments, the mammalian pluripotent stem cells (e.g., ESCs) are cultured in the FC media for about 3 days.

In some embodiments, the method comprises co-culturing mammalian pluripotent stem cells (e.g., ESCs) in an in vitro culture (IVC) media, optionally following culturing the mammalian pluripotent stem cells (e.g., ESCs) in the FC media. The mammalian pluripotent stem cells (e.g., ESCs) can be cultured in the IVC media for 1, 2, 3, or 4 days. In some embodiments, the mammalian ESCs are cultured in the IVC media for about 2 days, optionally following culturing in the FC media for about 3 days.

In some embodiments, the method comprises partially replacing a quantity of a FC media (e.g., at least half of the media) with a refresh FC media or an IVC media. The replacement can occur every 20-28 hours of the culturing (e.g., every 24 hours). In some embodiments, the method comprises partially replacing a quantity of an IVC media (e.g., at least half of the media) with a refresh IVC media.

In some embodiments, co-culturing mammalian pluripotent stem cells (e.g., ESCs) in a FC media and/or an IVC media is performed under a static condition allowing the ESCs to self-assemble into a post-implantation gastrulating embryo structure.

In some embodiments, the method comprises culturing the post-implantation gastrulating embryo structure in a post-implantation culture medium under a condition allowing the gastrulating embryo structure to develop into a synthetic embryo. A post-implantation culture media is capable of supporting the development of an embryo ex utero. In some embodiments, the post-implantation culture media contains human cord serum. In some embodiments, the post-implantation culture media contains bicarbonate or HEPES.

Culturing a post-implantation gastrulating embryo structure in a post-implantation culture medium can be performed under a static or dynamic condition. For example, culturing a post-implantation gastrulating embryo structure can comprise culturing the post-implantation gastrulating embryo structure in the post-implantation culture medium under a static condition, followed by culturing in the post-implantation culture medium under a dynamic condition. The post-implantation embryo structure can be cultured in the post-implantation culture media for 3 or more days (e.g., 1, 2, 3 or more days). For example, the method can comprise culturing the gastrulating embryo structure under a static condition for a duration of about 2 days, followed by culturing the gastrulating embryo structure under the dynamic condition for a duration of at least one day. The volume of the culture media can be increased every 20-28 hours (e.g., every 24 hours) by feeding the embryos with an amount of fresh culture media (e.g., 200-500 μl per embryo).

The culture media disclosed herein, including the FC media, IVC media and the post-implantation culture medium, can comprise a basal culture medium. The basal medium may comprise water, salts, amino acids, a carbon source, vitamins, lipids and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. The basal culture medium can comprise Dulbecco's Modified Eagle Medium (DMEM), DMEM Nutrient Mixture 12 (DMEM/F12), a non-human serum or serum substitute thereof, an antibiotic, L-glutamine or an analogue thereof (e.g., GlutaMAX™), or any combination thereof.

The non-human serum or serum substitute can comprise fetal bovine serum, bovine serum albumin, rat serum, KnockOut™ Serum Replacement, or any combination thereof. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof.

In some embodiments, the culture media (e.g., FC media and IVC media) comprises a reducing agent. The reducing agent can comprise beta-mercaptoethanol (BME), N-acetyl-L-cysteine, dithiothreitol (DTT), or any combination thereof.

The concentration or amount of one or more of the components in a solution or media can vary. The amount of, e.g., the non-human serum or serum substitute thereof, antibiotic, reducing agent, and/or L-glutamine (e.g., GlutaMax™) can vary, and, in some embodiments, can be adjusted as needed by one of skill in the art. In some embodiments, the amount of non-human serum or serum substitute thereof can comprise about 0.01% to about 40% (e.g., about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the amount of antibiotic can comprise about 0.01% to about 10% (e.g., about 0.01%, 0.1%, 1%, 2%, 3%, 4%, 5%, 6%, 7%, 8%, 9%, 10% or a number or a range between any two of these values) volume per volume (% v/v), weight per volume (% w/v) or weight per weight (% w/w) of the medium. The amount of e.g., the reducing agent can vary. For example, in some embodiments, the concentration of the reducing agent in the composition can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 μM, 1 mM, or a number or a range between any two of these values). The amount of L-glutamine (e.g., GlutaMAX™) can vary. For example, in some embodiments, the concentration of L-glutamine in the culture media can be about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. Where percentages are provided for agents, ingredients and compounds, they can be % w/w, % w/v or % v/v with respect to the formulation as a whole, unless otherwise indicated.

Each component of the culture medium described herein may be present in an amount such that the culture medium is suitable for supporting the self-assembly of stem cells (e.g., ESCs) into a post-implantation embryo structure and/or further development of the post-implantation embryo structure.

In the in vitro culture medium embodiments defined herein, the culture medium may be free, substantially free or essentially free of one or more of an epidermal growth factor (EGF) receptor agonist or an analogue thereof, such as EGF or an EGF substitute; a fibroblast growth factor family (FGF) receptor agonist or an analogue thereof, such as FGF or an FGF substitute; a Leukemia Inhibitory Factor (LIF) receptor agonist or an analogue thereof, such as LIF or a LIF substitute; a Bone Morphogenic Protein (BMP) receptor agonist or an analogue thereof, such as a BMP, or a BMP substitute; a WNT receptor agonist or an analogue thereof, such as WNT or a WNT substitute. The culture medium further may be free, substantially free or essentially free of a TGFβ receptor agonist or an analogue thereof. Unless otherwise indicated, the culture medium further may be free, substantially free or essentially free of nodal, activin, stem cell factor or members of the hedgehog family of proteins.

The mammalian pluripotent stem cells described herein, e.g., the wild type and ESCs with transcription factor overexpression, can be individually cultured, prior to the co-culturing described herein, in a suitable culture media suitable for stem cell and pluripotent stem cell proliferation as will be understood by a person skilled in the art. For example, the ESCs can be cultured in a culture medium free of serum or substantially free of serum or essentially free of serum. The culture medium may comprise a serum replacement medium. Such serum replacement media are commercially available under the trade names KSR (KnockOut™ Serum Replacement, Invitrogen, 10828-010) and N2B27 (e.g., Invitrogen, ME100137L1). The serum replacement medium may be included in the culture medium at about 5% to about 60%, about 10% to about 50%, about 15% to about 45%, or about 20% to about 40%.

The culture medium described herein may contain other components, or analogues thereof. As used herein, the term “analogue” can refer to a biologically active analogue of any of the components of the culture medium. Such an analogue may be natural or synthetic.

The specific biologically active ligands and compounds used in the media defined herein, such as insulin, progesterone, etc. are used for illustrative purposes. However, one of skill in the art will readily recognize that analogues of such ligands and compounds may equally be used as alternatives, provided that they retain the relevant biological activity. One of skill in the art will be able to identify, in a routine manner, other biologically active compounds that are suitable for use as substitutes. For instance, these may be naturally occurring compounds or compounds which can be made by synthetic or semi-synthetic methods.

FC Media

In some embodiments, a FC media can further comprise an effective amount of sodium pyruvate, such as, for example, at a concentration of about 0.05 mM to about 20 mM (e.g., about 0.05, 0.1, 0.2, 0.3, 0.4, 0.5, 1, 1.5, 2, 2.5, 3, 3.5, 4, 4.5, 5, 6, 7, 8, 9, 10, 11, 12, 13, 14, 15, 16, 17, 18, 19, 20 mM or a number or a range between any two of these values). In some embodiments, the FC media comprises about 1 mM sodium pyruvate.

The FC media can also comprise an effective amount of an amino acid selected from the group comprising L-glycine, L-alanine, L-asparagine, L-aspartic acid, L-glutamic acid, L-proline and L-serine. The non-essential amino acids can have an effective amount of, for example, about 0.1% to about 2% (e.g., about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, or a number or a range between any two of these values) volume per volume (% v/v), weight per volume (% w/v) or weight per weight (% w/w) of the medium. In some embodiments, the FC media comprises about 1% non-essential amino acids. Non-essential amino acids may be included in the culture medium, for example, comprising glycine (about 1 mg/ml to about 25 mg/ml or about 5 mg/ml to about 10 mg/ml e.g., about 7.5 mg/ml), L-alanine (about 1 mg/ml to about 25 mg/ml or about 5 mg/ml to about 10 mg/ml e.g., about 9 mg/ml), L-asparagine (about 5 mg/ml to about 30 mg/ml or about 10 mg/ml to about 15 mg/ml e.g., about 13.2 mg/ml), L-aspartic acid (about 5 mg/ml to about 30 mg/ml or about 10 mg/ml to about 15 mg/ml e.g., about 13 mg/ml), L-glutamic acid (about 5 mg/ml to about 50 mg/ml or about 10 mg/ml to about 20 mg/ml e.g., about 15 mg/ml), L-proline (about 5 mg/ml to about 30 mg/ml or about 10 mg/ml to about 15 mg/ml e.g., about 11 mg/ml) and/or L-serine (about 5 mg/ml to about 30 mg/ml or about 10 mg/ml to about 15 mg/ml e.g., about 11 mg/ml). In some embodiments, culture medium may comprise L-glycine at a concentration of about 7.5 mg/ml, L-alanine at a concentration of about 9 mg/ml, L-asparagine at a concentration of about 13 mg/ml, L-aspartic acid at a concentration of about 13 mg/ml, L-glutamic acid at a concentration of about 14.5 mg/ml, L-proline at a concentration of about 11.5 mg/ml and L-serine at a concentration of about 10.5 mg/ml.

The non-human serum in the FC media can vary. In some embodiments, the FC media can comprise a non-human serum (e.g., fetal bovine serum) at about 5% to about 40% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the FC media comprises about 15% to about 20% non-human serum (e.g., fetal bovine serum). In some embodiments, the FC media comprises about 15% fetal bovine serum. In some embodiments, the FC media comprises about 20% fetal bovine serum.

The FC media can comprise an effective amount of L-glutamine or an analogue thereof. L-glutamine may be included in the culture medium at a concentration of about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. In some embodiments, L-glutamine is included in the FC medium at a concentration of about 2 mM.

The FC media can comprise an effective amount of a reducing agent. In some embodiments, the concentration of the reducing agent in the FC media can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 μM, 1 mM, or a number or a range between any two of these values). In some embodiments, the reducing agent is included in the FC media at a concentration of about 0.1 mM. In some embodiments, the FC media comprises β-mercaptoethanol (BME) at a concentration of about 0.1 mM.

In some embodiment, the FC media comprises an effective amount of an inhibitor of rho-associated protein kinase (ROCK) (also referred to herein as ROCK inhibitor). Exemplary ROCK inhibitors include, but are not limited to N-[(1S)-2-Hydroxy-1-phenylethyl]-N′-[4-(4-pyridinyl)phenyl]-urea (AS1892802), fasudil hydrochloride (also known as HA 1077), -[3-[[2-(4-Amino-1,2,5-oxadiazol-3-yl)-I-ethyl-1H-imidazo[4,5-c]pyridin-6-yl]oxy]phenyl]-4-[2-(4-morpholinyl)ethoxy]benzamide (GSK269962), 4-[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (S)-(+)-2-Methyl-1-[(4-methyl-5-isoquinolinyl)sulfonyl]-hexahydro-1H-1,4-diazepine dihydrochloride (H 1152 dihydrochloride), (S)-(+)-4-Glycyl-2-methyl-1-[(4-methyl-5-isoquinolinyl)sulfonyl]-hexahydro-1H-1,4-diazepine dihydrochloride (glycyl-H 1152 dihydrochloride), N-[(3-Hydroxyphenyl)methyl]-N′-[4-(4-pyridinyl)-2-thiazolyl]urea dihydrochloride (RKI 1447 dihydrochloride), (3S)-1-[[2-(4-Amino-1,2,5-oxadiazol-3-yl)-1-ethyl-1H-imidazo[4,5-c]pyridin-7-yl]carbonyl]-3-pyrrolidinamine dihydrochloride (SB772077B dihydrochloride), N-[2-[2-(Dimethylamino)ethoxy]-4-(1H-pyrazol-4-yl)phenyl-2,3-dihydro-1,4-benzodioxin-2-carboxamide dihydrochloride (SR 3677 dihydrochloride), and trans-4-[(R)-1-Aminoethyl]-N-4-pyridinylcyclohexanecarboxamide dihydrochloride (Y-27632 dihydrochloride), N-Benzyl-[2-(pyrimidin-4-yl)amino]thiazole-4-carboxamide (Thiazovivin), Rock Inhibitor, a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody commercially available from Santa Cruz Biotechnology selected from the group consisting of Rock-1 (B 1), Rock-1 (C-19), Rock-1 (H-11), Rock-1 (G-6), Rock-1 (H-85), Rock-1 (K-18), Rock-2 (C-20), Rock-2 (D-2), Rock-2 (D-11), Rock-2 (N-19), Rock-2 (H-85), Rock-2 (30-J); a ROCK CRISPR/Cas9 knockout plasmid selected from the group consisting of Rock-1 CRISPR/Cas9 KO plasmid (h), Rock-2 CRISPR/Cas9 KO plasmid (h), Rock-1 CRISPR/Cas9 KO plasmid (m), Rock-2 CRISPR/Cas9 KO plasmid (m); a ROCK siRNA, shRNA plasmid and/or shRNA lentiviral particle gene silencer selected from the group consisting of Rock-1 siRNA (h): sc-29473, Rock-1 siRNA (m): sc-36432, Rock-1 siRNA (r): sc-72179, Rock-2 siRNA (h): sc-29474, Rock-2 siRNA (m): sc-36433, Rock-2 siRNA (r): sc-108088. In some embodiments, the ROCK inhibitor comprises Y-27632.

The FC media can comprise an effective amount of ROCK inhibitor, such as, for example, at a concentration of about 0.1 nM to about 100 nM (e.g., about 0.1, 0.2, 0.3, 0.4, 0.5, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mM or a number or a range between any two of these values). In some embodiments, the FC media comprises a ROCK inhibitor at a concentration of about 7.5 nM. In some embodiments, the FC media comprises about 1 nM to about 100 nM Y-27632 (e.g., about 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nM or a number or a range between any two of these values). In some embodiments, the FC media comprises about 7.5 nM Y-27632. In some embodiments, the FC media does not comprise a ROCK inhibitor.

In some embodiments, the FC media comprises DMEM, fetal bovine serum, sodium pyruvate, GlutaMax™, MEM non-essential amino acids, 2-mercaptoethanol, penicillin and/or streptomycin, or any combination thereof.

In some embodiments, the FC media comprises DMEM, about 15% fetal bovine serum, about 1 mM sodium pyruvate, about 2 mM GlutaMax, about 1% MEM non-essential amino acids, about 0.1 mM 2-mercaptoethanol, about 1% penicillin and/or streptomycin, or any combination thereof.

In some embodiments, the FC medium comprises Dulbecco's modified essential medium (Gibco 41966052), about 15% fetal bovine serum (Cambridge Stem Cell Institute), about 1 mM sodium pyruvate (Gibco 11360039), about 2 mM GlutaMAX (Gibco 35050038), about 1% MEM non-essential amino acids (Gibco 11140035), about 0.1 mM 2-mercaptoethanol (Gibco 31350010) and about 1% penicillin/streptomycin (Gibco 15140122).

In some embodiments, the FC media further comprises an anticoagulant, optionally heparin, a fibroblast growth factor (FGF), optionally FGF2 and/or FGF4, or any combination thereof. For example, the FC media can be supplemented with heparin and FGF4 on or prior to the first day of the ESC co-culturing. In some embodiments, the FC media can be supplemented with about 1 μg/mL heparin and 25 ng/mL FGF4.

IVC Media

The IVC media contains a basal medium described herein. The basal medium can comprise water, salts, amino acids, a carbon source, vitamins, lipids and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. Basal media are commercially available, for example, under the trade names Advanced DMEM/F12 (Gibco, 12634-010) and CMRL-1066 (Invitrogen or Sigma). The basal culture medium can comprise Dulbecco's Modified Eagle Medium (DMEM), DMEM Nutrient Mixture 12 (DMEM/F12), Roswell Park Memorial Institute (RPMI) medium 1640, Neurobasal®, Neurobasal® A, Connaught Medical Research Laboratory 1066 (CMRL-1066), or any combination thereof.

An IVC media can further comprise (a) insulin, an insulin analogue, or an insulin receptor agonist; (b) estrogen, an estrogen analogue, or an estrogen receptor agonist; and (c) progesterone, a progesterone analogue, or a progesterone receptor agonist.

The amount of the insulin, estrogen, progesterone, or analogues or receptor agonists thereof present in the IVC media can vary. For example, in some embodiments, the IVC media can comprise about 1 ng/ml to about 100 mg/ml (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 ng/ml, 1 μg/ml, 10, 20, 30, 40, 50, 60, 70, 80, 90, 100 μg/ml, 1 mg/ml, 10, 20, 30, 40, 50, 60, 70, 80, 90, 100 mg/ml or a number or a range between any two of these values) of one or more hormones (e.g., progesterone) and/or one or more growth factors (e.g., insulin or an insulin-like growth factor). In some embodiments, the IVC media can comprise about 0.5 nM to about 1 mM (e.g., about 0.5, 1, 1.5, 2, 2.5, 3, 3.5, 4, 4.5 5 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900 nM, 0.5 mM, 1 mM, or a number or a range between any two of these values) of a hormone (e.g., estrogen) and/or insulin or an insulin-like growth factor.

In some embodiments, the insulin receptor agonist is selected from the group comprising IGF-I, IGF-II, analogues thereof, or any combination thereof. The estrogen receptor agonist can be selected from the group comprising β-estradiol, estrone, estriol and estetrol, or any analogue thereof. The IVC media can comprise transferrin, sodium selenium, ethanolamine, or any analogue thereof. The IVE media can comprise Insulin-Transferrin-Selenium-Ethanolamine (ITS-X). In some embodiments, the IVC media further comprises an agonist of the activin type 1 or type 2 receptors. The IVC media can comprise a reducing agent. In some embodiments, the reducing agent can comprise N-acetyl-L-cysteine, dithiothreitol (DTT), β-mercaptoethanol (BME), or any combination thereof.

The IVC media can comprise a non-human serum, the concentration of which can vary in different embodiments. In some embodiments, the IVC media can comprise a non-human serum (e.g., fetal bovine serum) at about 5% to about 40% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the IVC media comprises about 20% to about 30% non-human serum (e.g., fetal bovine serum). In some embodiments, the IVC media comprises about 20% fetal bovine serum. In some embodiments, the IVC media comprises about 30% fetal bovine serum. In some embodiments, culturing the mammalian pluripotent stem cell and the extra-embryonic stem cell in the IVC media comprises increasing serum concentrations, optionally increasing the serum concentration from about 20% to about 30%.

The IVC media can comprise L-glutamine. L-glutamine may be included in the culture medium at a concentration of about 0.1 mM to about 40 mM, about 0.2 mM to about 20 mM, about 0.5 mM to about 10 mM, about 1 mM to about 5 mM or about 1.5 mM to about 2.5 mM e.g., about 2 mM. In some embodiments, L-glutamine is included in the culture medium at a concentration of about 2 mM.

The IVC media can comprise an effective amount of a reducing agent. In some embodiments, the concentration of the reducing agent in the IVC media can be about 0.1 μM to about 1 mM (e.g., about 0.1, 0.2, 0.3, 0.4, 0.5, 0.6, 0.7, 0.8, 0.9, 1, 10, 100, 200, 300, 400, 500, 600, 700, 800, 900 μM, 1 mM, or a number or a range between any two of these values). In some embodiments, the reducing agent is included in the IVC media at a concentration of about 25 μM. In some embodiments, the IVC media comprises N-acetyl-L-cysteine at a concentration of about 25 μM.

Penicillin may be included in the culture medium at a concentration of about 1 unit/ml to about 500 units/ml, about 2 units/ml to about 250 units/ml, about 5 units/ml to about 100 units/ml, about 10 units/ml to about 50 units/ml, or about 20 units/ml to about 30 units/ml e.g., about 25 units/ml. Streptomycin may be included in the culture medium at a concentration of about 1 μg/ml to about 500 μg/ml, about 2 μg/ml to about 250 μg/ml, about 5 μg/ml to about 100 μg/ml, about 10 μg/ml to about 50 μg/ml, 25 or about 20 μg/ml to about 30 μg/ml e.g., about 25 μg/ml. The culture medium can comprise penicillin at a concentration of about 25 units/ml and/or streptomycin at a concentration of about 25 μg/ml.

The culture medium may comprise a basal medium, as defined above, (e.g., Advanced DMEM/F12) supplemented with, an insulin receptor agonist, e.g., Insulin (e.g., about 2 mg/ml to about 25 mg/ml), Transferrin (e.g., about 1 mg/ml to about 10 mg/ml), Selenium e.g., sodium selenite (e.g., about 0.001 mg/ml to about 0.01 mg/ml), Ethanolamine (e.g., about 0.5 mg/ml to about 10 mg/ml), an estrogen receptor agonist e.g., estradiol (e.g., about 5 nM to about 10 nM), a progesterone receptor agonist e.g., Progesterone (e.g., about 50 ng/ml to about 500 ng/ml) and a reducing agent e.g., N-acetyl-L-cysteine (e.g., about 17.5 μM to about 40 μM). In some embodiments, the IVC media is free or substantially free of sodium pyruvate.

In some embodiments, the IVC media comprises DMEM/F12, fetal bovine serum, GlutaMax, ITS-X, β-estrodiol, progesterone, N-acetyl-L-cysteine, penicillin and/or streptomycin, or any combination thereof.

In some embodiments, the IVC media comprises DMEM/F12, about 20% fetal bovine serum, about 2 mM GlutaMax, about 1×ITS-X, about 8 nM β-estrodiol, about 200 ng/ml progesterone, about 25 μM N-acetyl-L-cysteine, about 1% penicillin and/or streptomycin, or any combination thereof.

Post-Implantation Media

In some embodiments, the post-implantation gastrulating embryo is cultured in a post-implantation culture medium capable of supporting the development of embryos ex utero. The method described herein comprises culturing the post-implantation gastrulating embryo structure in a post-implantation culture medium under a condition allowing the post-implantation gastrulating embryo structure to develop into a synthetic embryo. The condition can comprise a static cell culture condition (e.g., for about 2 days) followed by a dynamic cell culture condition (e.g., for at least 1 day).

In some embodiments, the post-implantation culture medium comprises a basal medium described herein. The basal medium can comprise Dulbecco's Modified Eagle Medium (DMEM) or DMEM Nutrient Mixture 12 (DMEM/F12), non-human serum (e.g., rat and/or bovine serum), human cord serum, L-glutamine or an analogue thereof (e.g., GlutaMAX™), antibiotics, or any combination thereof.

The DMEM or DMEM/F12 can be present in the media at about 5% to about 40% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the DMEM or DMEM/F12 is present in the media at about 25%. In some embodiments, the post-implantation culture medium comprises DMEM at about 25%.

The post-implantation culture medium comprises an effective amount of non-human serum at about 5% to about 60% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, or a number or a range between any two of these values) volume per volume (% v/v), weight per volume (% w/v) or weight per weight (% w/w) of the medium. In some embodiments, the post-implantation culture medium comprises about 50% non-human serum. In some embodiments, the post-implantation culture medium comprises about 50% rat serum.

The human cord serum in the post-implantation culture medium can vary. In some embodiments, the post-implantation culture medium comprises an effective amount of human cord serum at about 5% to about 40% (e.g., 5%, 10%, 11%, 12%, 13%, 14%, 15%, 16%, 17%, 18%, 19%, 20%, 21%, 22%, 23%, 24%, 25%, 26%, 27%, 28%, 30%, 31%, 32%, 33%, 34%, 35%, 36%, 37%, 38%, 39%, 40%, or a number or a range between any two of these values) volume per volume (% v/v), weight per volume (% w/v) or weight per weight (% w/w) of the medium. In some embodiments, the post-implantation culture medium comprises about 25% human cord serum.

In some embodiments, the post-implantation culture medium comprises about 25% DMEM, about 50% rat serum, and about 25% human cord serum.

In some embodiments, the post-implantation culture medium further comprises an effective amount of bicarbonate. The bicarbonate can be present in the post-implantation media at a concentration from about 0.1 mM to about 30 mM, optionally from 1 mM to about 20 mM.

In some embodiments, the post-implantation culture medium comprises an effective amount of HEPES. The HEPES can be present in the post-implantation culture medium at a concentration from about 0.1 mM to about 30 mM, optionally from 1 mM to about 20 mM.

The post-implantation culture medium can comprise an effective amount of L-glutamine or an analogue thereof (e.g., GlutaMAX™). L-glutamine may be included in the culture medium at a concentration of about 0.1 mM to about 40 mM, about 0.2 mM to about 20 mM, about 0.5 mM to about 10 mM, about 1 mM to about 5 mM or about 1.5 mM to about 2.5 mM e.g., about 2 mM.

The antibiotics can be include in the post-implantation culture medium at a concentration of about 1 unit/ml to about 500 units/ml. For example, penicillin may be included in the culture medium at a concentration of about 1 unit/ml to about 500 units/ml, about 2 units/ml to about 250 units/ml, about 5 units/ml to about 100 units/ml, about 10 units/ml to about 50 units/ml, or about 20 units/ml to about 30 units/ml e.g., about 25 units/ml. Streptomycin may be included in the culture medium at a concentration of about 1 μg/ml to about 500 μg/ml, about 2 μg/ml to about 250 μg/ml, about 5 μg/ml to about 100 μg/ml, about 10 μg/ml to about 50 μg/ml, 25 or about 20 μg/ml to about 30 μg/ml e.g., about 25 μg/ml. The post-implantation culture medium can comprise penicillin at a concentration of about 100 units/ml and/or streptomycin at a concentration of about 100 μg/ml.

In some embodiments, the post-implantation culture medium comprises DMEM, rat serum, human cord serum, GlutaMax, penicillin and/or streptomycin, HEPES, or any combination thereof.

In some embodiments, the post-implantation culture medium comprises about 25% DMEM, about 50% rat serum, and about 25% human cord serum. The post-implantation culture medium is further supplied with 1× Glutamax (GIBCO, 35050061), 100 units/ml penicillin and 100 μg/ml streptomycin and 11 mM HEPES (GIBCO 15630056).

In some embodiments, culturing the post-implantation embryo comprises supplying the post-implantation culture medium with glucose. The glucose can be provided in the post-implantation culture medium or in the basal cultural medium in an constant concentration or in increasing concentrations throughput the culturing. In some embodiments, the glucose is provided at a constant concentration at about 3 mg/ml. In some embodiments, the glucose concentration can be increased from at least about 3 mg/ml up to about 4-5 mg/ml. In some embodiments, the glucose is supplied to the post-implantation culture medium from about 3 mg/ml up to about 3.5 mg/ml. The glucose can be supplied for at least once (e.g., one, two, three or more times) at a concentration of at least 3 mg/ml (e.g., 3, 3.5, 4, 4.5, 5, or higher). The post-implantation culture medium can be supplied with a glucose of at least 3 mg/ml at about day three of culturing in the post-implantation culture medium.

In some embodiments, the glucose is provided in the post-implantation culture medium in increasing concentrations throughout the culturing. In some embodiments, the glucose is provided in the post-implantation culture medium in a constant concentration followed by increasing concentrations. In some embodiments, the glucose is provided in the post-implantation culture medium in a constant concentration throughout a static condition followed by increasing concentrations throughout a dynamic condition. In some embodiments, the glucose can be provided in the post-implantation culture medium at a constant concentration under a static condition followed by increasing concentrations in the same or different static concentration. In some embodiments, the glucose is not provided in the post-implantation culture medium when culturing under a static condition and subsequently supplemented to the post-implantation culture medium at a constant concentration when culturing under a dynamic condition. For example, culturing the post-implantation embryo can comprise culturing, under a static condition, the gastrulating embryo structure in a culture medium that does not contain glucose for about two days, followed by culturing, under a dynamic condition, the post-implantation embryo structure in a culture medium comprising about 3 mg/ml glucose for about one day.

### An Embryo Culturing System

In some embodiments, cultures starting at post-implantation or later stages such as a neurulating stage can be incubated in an embryo culturing device. Accordingly, provided herein also includes an embryo culturing device or system for incubating a synthetic embryo structure described herein in vitro. In some embodiments, the embryo culturing system can comprise a rotating incubator comprising a culture medium described herein (e.g., a post-implantation culture medium), a plurality of gas cylinders or gas sources, and a gas mixer configured to receive gases from the plurality of gas cylinders. In some embodiments, the device also comprises a gas and pressure controller for controlling and monitoring the content percentage, pressure and/or humidity of the gases in the system. In some embodiments, the gas mixer and a gas and pressure controller can be comprised in a gas control system. The gases from the gas cylinders are delivered into the gas mixer, in which the gases are mixed to a desired ratio, pressure and/or humidity level. The gas mixture generated from the gas mixer is then fed into the rotating incubator (e.g., rotating bottles). The gas mixture can be humidified before being fed into the rotating incubator. The pressures for each gas entering the gas mixer, exiting the gas mixer and entering the rotating incubator can vary in different embodiments. One or more of the components described here may be commercially available.

An embryo culturing device or system described herein comprises a plurality of gas cylinders. A gas cylinder is a pressure vessel for storage and containment of gases, optionally at above atmospheric pressure (e.g., above 1 psi). The gas cylinder may be connected to one or more gas sources, each of which contains a type of gas (e.g., O₂, CO₂, or N₂). The gas cylinder may be connected to a gas and pressure controller configured to manipulate and monitor the gases in the systems, such as for controlling the pressure and concentration of the gases. For example, in regular air there is about 21% oxygen, and when lower levels of oxygen are required inside the culturing system, then nitrogen gas is delivered in order to reduce the levels of oxygen in the system. In some embodiments, when higher concentrations of oxygen are required, nitrogen gases are stopped, and pure oxygen is provided. In some embodiments, the system is configured to provide any combination of mixture of gases. In some embodiments, a plurality of gas cylinders described herein comprises an O₂ gas cylinder, a CO₂ gas cylinder, and a N₂ gas cylinder. The gases may be accumulated at a pressure pump until a predetermined level of pressure is reached before they are delivered into the gas mixer. In some embodiments, the pressure pump is configured to provide mixed gases at a pressure of from about 1 psi to about 15 psi, optionally from 2 psi to 14 psi, or from 3 psi to 13 psi, from 2 psi to 12 psi, 3 psi to 11 psi, 4 psi to 10 psi, 5 psi to 11 psi, 6 psi to 12 psi, 7 psi to 13 psi, 9 psi to 14 psi, or 10 psi to 15 psi. In some embodiments, the mixed gases are injected to the gas mixer at an input pressure of about 10 psi.

An embryo culturing device or system also comprises a gas mixer. A gas mixer can be part of a gas control system that ensures complete and uniform mixing of different gases required to provide a necessary environment in the containers of an incubator. The size of the gas mixer can vary in different embodiments. In some embodiments, the gas mixer comprises an internal volume of from about 250,000 cm³ to about 260,000 cm³.

In some embodiments, the gas mixer provides two streams of any mix of oxygen and carbon dioxide gas without the use of premixed gas cylinders. The flow rate for each stream is independently controlled for gas efficiency to provide a reproducible rate at which the gas is delivered to the cell culture chamber and/or diffused into the cell culture media. By independently monitoring and controlling oxygen and carbon dioxide concentrations of the streams, the gas control system can monitor and proportionally mix oxygen, carbon dioxide and nitrogen gases into any oxygen and carbon dioxide concentration in real time. In some embodiments, the gas mixer comprises gas sensors configured to monitor the concentrations, content percentages, and/or pressures of the gases inside the gas mixer. For example, the gas mixer can comprise two sensors, an O₂ sensor and a CO₂ sensor, for monitoring the content percentage of O₂ and CO₂, respectively. For example, after calibrating the sensors, the desired concentrations for O₂ (0.1-99.9%) and CO₂ (0.1-20.0%) are entered into their respective controllers. The closed-loop dynamic gas control system will then automatically monitor and adjust the output stream of gas to the pre-programmed gas ratios in the controllers. This technique can maintain the output mixed gas concentration to within 0.2% of the selected setpoint levels. In some embodiments, the gas mixer comprises O₂ at a content percentage from 5% up to 6%, 7%, 8%, 9%, 10%, 11%, 12%, 13%, 14%, 15%, 16%, 17%, 18%, 19%, 20%, 21%, 22%, 23%, 24%, 25% or higher. In some embodiments, the gas mixer comprises CO₂ at a content percentage from 1% up to 2%, 3%, 4%, 5%, 6%, 7%, 8%, 9%, 10% or higher. In some embodiments, the gas mixer comprises about 21% O₂ and about 5% CO₂. In some embodiments, the gas mixer can comprise a humidifier comprising a container with at least one liquid, such as water. The mixed gases at a desired pressure, content percentages, and a humidity level are then delivered to the rotating incubator. In some embodiments, the mixed gases is delivered to the rotating incubator at a pressure less than 1 psi, such as about 0.5 psi. An exemplar gas mixer is the OxyStreamer from Biospherix.

An embryo culturing device or system herein described also comprises a rotating incubator that can be used in a dynamic (e.g., rotating) culture method described above. In some embodiments, the rotating incubator comprises a rotating bottle culture unit. A rotating culturing method provides a continuous flow of oxygenating gas to culture in rotating bottles. In some embodiments, culture bottles in the rotating bottle culture unit are plugged into a hollow rotating drum. Oxygenating gas flows along the axis and is distributed to the culture bottles by a baffle plate within the drum. In some embodiments, the system maintains a stable pH, when compared to other systems with sealed culture bottles. In some embodiments, the rotator is supplied complete with gas filter, bubbler and leads by the manufacturer. In some embodiments, a rotating incubator, such as the BTC Precision Incubator from B.T.C. Engineering,—Cullum Starr Precision Engineering Ltd—UK, uses a thyristor-controlled heater and high flow-rate fan to give a highly stable and uniform temperature throughout the easily accessible working volume. In some embodiments, the incubator has a working volume 370×350×200 mm high which is accessed through a hinged top. In some embodiments, the heater element is rated at 750 Watts. In some embodiments, Bung (Hole) BTC 06 is used to seal the bottles and Bung (Solid) BTC 07 is used to seal the drum. An exemplary rotating bottle culture unit is described and illustrated in www.cullumstarr.com/btc-engineering/rotating-bottle-culture-unit.

In some embodiments, the mixed gases are delivered into a bottle that can be partially filled with a liquid such as water, thereby creating bubbles in the liquid. The bubble bottle allows a user to visualize that the system is delivering mixed gases by assessing the flow rate of the bubbles. The bubble rate (e.g., bubbles/sec) can indicate the speed of gas flowing through the bottles. In some embodiments, the flow rate at the exit of the rotating bottle system is about 8-10 bubbles/sec. In some embodiments, the liquid-filled bottle can work as a humidifier for the gases. Alternatively or in addition, the mixed gases can optionally delivered into an additional humidifier.

In some embodiments, the mixed gases are delivered into a rotating drum (e.g., from a humidifier), which comprises containers containing embryos and culture medium necessary for the growth and/or maintenance of the embryos. The rotating drum can be configured to rotate at velocity of from about 1 rpm to about 100 rpm. In some embodiments, the rotating bottles can be rolled at 1-10 rpm, about 2-8 rpm, 3-6 rpm, or about 3 rpm. Medium in the rotating bottles (e.g., the post-implantation medium) can be changed by taking out each container and carefully changing the medium according to known techniques Medium in the rotating bottles can be changed or refreshed every 24 hours with minimum disturbance to the embryos.

### Applications

Provided herein also includes a synthetic embryo obtainable by the in vitro method described herein for use in a method of diagnosing, preventing or treating a disease in a patient in need thereof. For example, embryo cells obtainable from the present invention may be used in stem cell therapies, such as treatments for cancers, replacement tissue, reconstructive surgery, tissue repair, wound healing, bone marrow transplantation, stroke, baldness, blindness, deafness, diabetes, heart disease, bowel disease, arthritis, skeletal injury, teeth replacement, neuronal disease and any other condition where replacement cells or tissues may be advantageous. The cells may also be utilized for screening therapeutic compounds for efficacy and safety, as would be understood by a person of skill in the art.

In some embodiments, the synthetic embryo structure for use in a method of diagnosing, preventing or treating a disease in a patient in need thereof as described herein may be used for transplantation into the patient. It is envisaged that in certain embodiments, the pluripotent stem cell used to obtain the embryo may have been obtained from the patient originally, thus reducing the likelihood of rejection by the patient's immune system. Thus, a pluripotent stem cell, for example an embryonic stem cell, obtained from a subject may be cultured using the methods described herein to provide material for transplantation back into that subject to prevent or treat a condition. For example, the embryo may be used to grow replacement organs or tissues for the subject to regain function of such organs or tissues in the subject following loss of function through degeneration, aging and/or disease.

Disclosed herein also includes a method of providing a transgenic non-human animal, comprising gestating an embryo derived from a cell cultured using an in vitro method described herein. Such transgenic non-human animals may be useful in drug screening or in the study of disease. For example, model animals may be produced to study specific conditions. It is envisaged that the methods provided herein could be used to more efficiently develop transgenic and chimeric embryos (which currently relies for example, on the labor-intensive process of harvesting blastocysts and manually replacing the inner cell mass).

Disclosed herein include methods for investigating the effect of a test agent on embryonic development. In some embodiments, the method comprises: a) generating a synthetic embryo using the method described herein; b) contacting the synthetic embryo with a test agent; and c) determining the effect of the test agent on the synthetic embryo. In some embodiments, the determining comprises comparing a phenotype or a genotype of the synthetic embryo in the presence of the test agent with the phenotype or genotype of the synthetic embryo in the absence of the test agent. The method can comprise contacting the mammalian pluripotent stem cell with the test agent during or following step (a) and prior to step (b), during or following step (b) and prior to step (c), or during or following step (c).

The method can comprise determining the subsequent effect on formation of a synthetic embryo at various developmental stages. The determining can be performed using any method known in the art. For example, the method can comprise recording one or more images of the embryo structure.

Disclosed herein include methods for investigating mechanisms involved in embryogenesis. In some embodiments, the method comprises any of the in vitro methods for generating a synthetic embryo structure at various developmental stages described herein. Investigating mechanism involved in embryogenesis can comprise any method known in the art. For example, said investigating can comprise investigating the effect of a test agent on embryonic development as described above. In some embodiments, investigating mechanisms involved in embryogenesis can comprise determining the effect of genetic perturbation(s) in the embryo structure.

The method may comprise recording a plurality of images of the synthetic embryo structure. The plurality of images may be recorded over a pre-determined period of time, thus illustrating the development of the embryonic structure over time. The imaging apparatus may comprise microscopy apparatus, suitable recording apparatus, and optionally image processing apparatus.

Typically, fluorescent markers, such as fluorescent dyes or fluorescent marker proteins, are used in the imaging of embryonic development. Such markers may be added to the culture system. For example, fluorescent dyes may be added to visualize particular molecules or cellular structures. For example, DAPI may be used to stain DNA or MitoTracker (Invitrogen) may be used to stain the mitochondria. Additionally or alternatively, the embryo structure may produce such fluorescent markers endogenously, e.g., it may contain one or more cells which express a fluorescent marker protein. Such cells may have been genetically modified in order to confer the ability to express such a marker protein. Thus, fluorescence imaging apparatus may be particularly suitable for the methods described. The imaging apparatus may thus comprise a fluorescence microscope, such as a confocal microscope, that can include but is not limited to wide field, scanning and spinning disc confocal, and light sheet microscope.

Confocal microscopes image a single point of a specimen at any given time but allow generation of two dimensional or three dimensional images by scanning different points in a specimen in a regular raster to provide image data which can be assembled into a two or three dimensional image. For example, scanning a specimen in a single plane enables generation of a two dimensional image of a slice through the specimen. A plurality or “stack” of such two dimensional images can be combined to yield a three dimensional image. Spinning disc confocal microscopy provides added advantages over confocal laser scanning microscopy. Additionally, light sheet microscopy can also provide good imaging of embryonic development.

Disclosed herein also includes a method of elucidating the role of a gene in embryo development, the method comprising obtaining a pluripotent stem cell and/or an extra-embryonic stem cell where the gene has been modified or knocked out and culturing the pluripotent stem cell and the extra-embryonic stem cell using the in vitro method described herein. Thus, the methods may aid in the development of treatments for conditions relating to embryo development, such as fertility treatment.

Disclosed herein also includes a method of imaging an embryo during development comprising culturing a mammalian pluripotent stem and an extra-embryonic stem cell or a mammalian synthetic embryo structure using the methods described herein, and recording an image of said embryo using an imaging apparatus. The image may be a two dimensional or three dimensional image. A plurality of images may be recorded of the same embryo. An imaging apparatus can comprise microscopy apparatus and suitable recording apparatus. An imaging apparatus may further comprise image processing apparatus. Additionally, an imaging apparatus may further comprise a fluorescent microscope. Additionally, or alternatively, an imaging apparatus may further comprise a confocal microscope.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1-5 described below.

### Cell Lines and Culture Conditions

The ESC lines used included: CAG-GFP mouse ESCs (ESCs with constitutive membrane GFP expression, derived from CAG-GFP reporter mice); CAG-GFP tetO-Cdx2 mouse ESCs (ESCs overexpressing Cdx2 upon Dox induction, generated in-house using methods described below); CAG-GFP tetO-Gata4 mouse ESCs (ESCs overexpressing Gata4 upon Dox induction generated as previously reported); Cer1-GFP tetoO-Gata4 mouse ESCs (ESCs with GFP expression under Cer1 promoter which overexpress Gata4 upon Dox induction, generated as previously reported); CD1 mouse ESCs; CD1 tetO-Gata4 mouse ESCs (ESCs overexpressing Gata4 upon Dox induction generated as previously reported); Confetti TSCs; mT/mG mouse ESCs (ESCs expressing membrane tdTomato, derived from mT/mG mice); and Sox2-Venus/Brachyury-mCherry/Oct4-Venus mouse ESCs.

Cell lines and other reagent or resources used are listed below in Table 3.

TABLE 3
KEY RESOURCES

| REAGENT or RESOURCE | SOURCE | IDENTIFIER |
|---|---|---|
| Antibodies | | |
| Goat polyclonal anti-AP-2 gamma | R & D Systems | Cat# AF5059; |
| | | RRID: AB_2255891 |
| Mouse monoclonal anti-AP-2gamma | Santa Cruz | Cat# sc-12762; |
| | Biotechnology | RRID: AB_667770 |
| Goat polyclonal anti-Brachyury | R & D Systems | Cat# AF2085; |
| | | RRID: AB_2200235 |
| Mouse monoclonal anti-Cdx2 | BioGenex | Cat# MU392-UC; |
| | | RRID: AB_2335627 |
| Rabbit monoclonal anti-Cdx2 | Abcam | Cat# ab76541; |
| | | RRID: AB_1523334 |
| Rat monoclonal anti-Cerberus 1 | R & D Systems | Cat# MAB1986; |
| | | RRID: AB_2275974 |
| Goat polyclonal anti-Dkk1 | R & D Systems | Cat# AF1096; |
| | | RRID: AB_354597 |
| Rat monoclonal anti-E-Cadherin | Thermo Fisher | Cat# 13-1900; |
| | Scientific | RRID: AB_2533005 |
| Rabbit polyclonal anti-TBR2/Eomes | Abcam | Cat# ab23345; |
| | | RRID: AB_778267 |
| Rabbit monoclonal anti-FoxA2/HNF3 | Cell Signaling | Cat# 8186; |
| | Technology | RRID: AB_10891055 |
| Rabbit polyclonal anti-GATA-4 | Santa Cruz | Cat# sc-9053; |
| | Biotechnology | RRID: AB_2247396 |
| Goat polyclonal anti-GATA-6 | R & D Systems | Cat# AF1700; |
| | | RRID: AB_2108901 |
| Rat monoclonal anti-GFP | Nacalai Tesque | Cat# GF090R; |
| | | RRID: AB_2314545 |
| Mouse monoclonal anti-eHAND | Santa Cruz | Cat# sc-390376 |
| | Biotechnology | |
| Rabbit polyclonal anti-Keratin18 | Sigma-Aldrich | Cat# SAB4501665; |
| | | RRID: AB_10746153 |
| Goat polyclonal anti-Lefty | R & D Systems | Cat# AF746; |
| | | RRID: AB_355566 |
| Mouse monoclonal anti-Myh2 | R & D Systems | Cat# MAB4470; |
| | | RRID: AB_1293549 |
| Mouse monoclonal anti-N-Cadherin | BD Biosciences | Cat# 610,920; |
| | | RRID: AB_2077527 |
| Goat polyclonal anti-Nkx2.5 | R & D Systems | Cat# AF2444; |
| | | RRID: AB_355269 |
| Mouse monoclonal anti-Oct-3/4 | Santa Cruz | Cat# sc-5279; |
| | Biotechnology | RRID: AB_628051 |
| Goat polyclonal anti-Otx2 | R & D Systems | Cat# AF1979; |
| | | RRID: AB_2157172 |
| Rabbit polyclonal anti-RFP | Rockland | Cat# 600-401-379; |
| | | RRID: AB_2209751 |
| Rabbit monoclonal anti-RUNX1/AML1 | Abcam | Cat# ab92336; |
| | | RRID: AB_2049267 |
| Rabbit polyclonal anti-Sox1 | Cell Signaling | Cat# 4194; |
| | Technology | RRID: AB_1904140 |
| Mouse monoclonal anti-Sox2 | Santa Cruz | Cat# sc-365823; |
| | Biotechnology | RRID: AB_10842165 |
| Rat monoclonal anti-Sox2 | Thermo Fisher | Cat# 14-9811-82; |
| | Scientific | RRID: AB_11219471 |
| Goat polyclonal anti-Sox17 | R & D Systems | Cat# AF1924; |
| | | RRID: AB_355060 |
| Donkey anti-Mouse IgG (H + L) Highly | Thermo Fisher | Cat# A-21202; |
| Cross-Adsorbed Secondary Antibody, | Scientific | RRID: AB_141607 |
| Alexa Fluor 488 | | |
| Donkey anti-Rabbit IgG (H + L) Highly | Thermo Fisher | Cat# A-21206; |
| Cross-Adsorbed Secondary Antibody, | Scientific | RRID: AB_2535792 |
| Alexa Fluor 488 | | |
| Donkey anti-Goat IgG (H + L) Cross- | Thermo Fisher | Cat# A-11055; |
| Adsorbed Secondary Antibody, Alexa | Scientific | RRID: AB_2534102 |
| Fluor 488 | | |
| Donkey anti-Mouse IgG (H + L) Highly | Thermo Fisher | Cat# A10037; |
| Cross-Adsorbed Secondary Antibody, | Scientific | RRID: AB_2534013 |
| Alexa Fluor 568 | | |
| Donkey anti-Rabbit IgG (H + L) Highly | Thermo Fisher | Cat# A10042; |
| Cross-Adsorbed Secondary Antibody, | Scientific | RRID: AB_2534017 |
| Alexa Fluor 568 | | |
| Donkey anti-Goat IgG (H + L) Cross- | Thermo Fisher | Cat# A-11057; |
| Adsorbed Secondary Antibody, Alexa | Scientific | RRID: AB_2534104 |
| Fluor 568 | | |
| Donkey anti-Mouse IgG (H + L) Highly | Thermo Fisher | Cat# A-31571; |
| Cross-Adsorbed Secondary Antibody, | Scientific | RRID: AB_162542 |
| Alexa Fluor 647 | | |
| Donkey anti-Rabbit IgG (H + L) Highly | Thermo Fisher | Cat# A-31573; |
| Cross-Adsorbed Secondary Antibody, | Scientific | RRID: AB_2536183 |
| Alexa Fluor 647 | | |
| Donkey anti-Goat IgG (H + L) Cross- | Thermo Fisher | Cat# A-21447; |
| Adsorbed Secondary Antibody, Alexa | Scientific | RRID: AB_2535864 |
| Fluor 647 | | |
| Donkey Anti-Rat IgG H & L (Alexa | Abcam | Cat# ab150155; |
| Fluor ® 647) preadsorbed antibody | | RRID: AB_2813835 |
| Bacterial and virus strains | | |
| 5-alpha competent E. coli | New England | C2987I |
| | Biolabs | |
| Chemicals, peptides, and recombinant | | |
| proteins | | |
| β-estradiol | Sigma-Aldrich | Cat# E8875-1G |
| CHIR99021 | Cambridge Stem | N/A |
| | Cell Institute | |
| Doxycycline | Sigma-Aldrich | Cat# D9891-5G |
| Fgf2 | Cambridge Stem | N/A |
| | Cell Institute | |
| Recombinant Mouse FGF-4 (aa 67-202) | R & D Systems | Cat# 7486-F4-025 |
| Heparin | Sigma-Aldrich | Cat# H3149-25KU |
| Insulin-transferrin-selenium- | ThermoFisher | Cat# 51,500-056 |
| ethanolamine | Scientific | |
| Leukemia inhibitory factor | Cambridge Stem | N/A |
| | Cell Institute | |
| N-acetyl-L-cysteine | Sigma-Aldrich | Cat# A7250 |
| PD0325901 | Cambridge Stem | N/A |
| | Cell Institute | |
| Progesterone | Sigma-Aldrich | Cat# P8783-1G |
| Y-27632 | STEMCELL | Cat# 72,304 |
| | Technologies | |
| Critical commercial assays | | |
| Gateway ™ BP Clonase ™ II Enzyme mix | Invitrogen | Cat# 11,789-100 |
| Gateway ™ LR Clonase ™ II Enzyme mix | Invitrogen | Cat# 11,791-100 |
| Lipofectamine ™ 3000 Transfection | Invitrogen | Cat# L3000001 |
| Reagent | | |
| RNeasy Mini Kit | Qiagen | Cat# 74,104 |
| SYBR Green PCR Master Mix | Applied Biosystems | Cat# 4,368,708 |
| Deposited data | | |
| Single-cell RNA sequencing data | This manuscript | Array Express: E- |
| | | MTAB-12140 |
| Code for analyzing single-cell RNA | This manuscript | https://doi.org/ |
| sequencing data | | 10.5281/ |
| | | zenodo.7021607 |
| Experimental models: Cell lines | | |
| Mouse: CAG-GFP ESCs | Rhee et al. (2006) | N/A |
| Mouse: CAG-GFP/tetO-mCherry/tetO- | This manuscript | N/A |
| Cdx2 ESCs | | |
| Mouse: CAG-GFP/tetO-mCherry/tetO- | Amadei et al., 2021 | N/A |
| Gata4 ESCs | | |
| Mouse: Cerl-GFP/tetO-Gata4 ESCs | Amadei et al., 2021 | N/A |
| Mouse: CD1 ESCs | Prof Jennifer | N/A |
| | Nichols (Stem | |
| | Cell Institute, | |
| | University of | |
| | Cambridge, UK) | |
| Mouse: CD1/tetO-Gata4 ESCs | Amadei et al. | N/A |
| | (2022) | |
| Mouse: Confetti TSCs | Prof Jennifer | N/A |
| | Nichols (Stem | |
| | Cell Institute, | |
| | University of | |
| | Cambridge, UK) | |
| Mouse: mT/mG mouse ESCs | Muzumdar et al. | N/A |
| | (2007) | |
| Mouse: Sox2-Venus/Brachyury- | Dr Jesse Veenvliet | N/A |
| mCherry/Oct4-Venus mouse ESCs | and Prof Bernhard | |
| | G. Herrmann (Max | |
| | Planck Institute | |
| | of Molecular | |
| | Cell Biology and | |
| | Genetics) | |
| Experimental models: | | |
| Organisms/strains | | |
| Mouse: CD-1 | Charles River | N/A |
| | Laboratories | |
| Oligonucleotides | | |
| PCR primer: Cdx2-attB forward: | This manuscript | N/A |
| GGGGACAAGTTTGTACAAAAAAGC | | |
| AGGCTTAATGTACGTGAGCTACCTT | | |
| CTGGAC (SEQ ID NO: 1) | | |
| PCR primer: Cdx2-attB reverse: | This manuscript | N/A |
| GGGGACCACTTTGTACAAGAAAGC | | |
| TGGGTTTCACTGGGTGACAGTGGA | | |
| GTTTAAAAC (SEQ ID NO: 2) | | |
| qPCR primer: Cdx2 forward: | Blij et al. (2015) | N/A |
| AAACCTGTGCGAGTGGATG | | |
| (SEQ ID NO: 3) | | |
| qPCR primer: Cdx2 reverse: | Blij et al. (2015) | N/A |
| TCTGTGTACACCACCCGGTA | | |
| (SEQ ID NO: 4) | | |
| qPCR primer: Elf5 forward: | This manuscript | N/A |
| ATTTCTACAGTCCGCTGGTGC | | |
| (SEQ ID NO: 5) | | |
| qPCR primer: Elf5 reverse: | This manuscript | N/A |
| ACATCACCGTGAAGACAAGTGG | | |
| (SEQ ID NO: 6) | | |
| qPCR primer: Gapdh forward: | Amadei et al. | N/A |
| CGTATTGGGCGCCTGGTCAC | (2021) | |
| (SEQ ID NO: 7) | | |
| qPCR primer: Gapdh reverse: | Amadei et al. | N/A |
| ATGATGACCCTTTTGGCTCC | (2021) | |
| (SEQ ID NO: 8) | | |
| qPCR primer: Gata3 forward: | This manuscript | N/A |
| TGTCTGCGAACACTGAGCTG | | |
| (SEQ ID NO: 9) | | |
| qPCR primer: Gata3 reverse: | This manuscript | N/A |
| CGATCACCTGAGTAGCAAGGAG | | |
| (SEQ ID NO: 10) | | |
| Recombinant DNA | | |
| PB-tetO-hygro | Dr José Silva | N/A |
| | (Stem Cell | |
| | Institute, | |
| | University of | |
| | Cambridge, UK) | |
| pBAse | Dr José Silva | N/A |
| | (Stem Cell | |
| | Institute, | |
| | University of | |
| | Cambridge, UK) | |
| rtTA-zeocyin | Dr José Silva | N/A |
| | (Stem Cell | |
| | Institute, | |
| | University of | |
| | Cambridge, UK) | |
| Software and algorithms | | |
| Fiji | Schindelin et | https:// |
| | al. (2012) | ImageJ.net/ |
| | | Fiji |
| metacell_0.3.7 | Baran et al. | https:// |
| | (2019) | github.com/ |
| | | tanaylab/ |
| | | metacell |
| Metacells 0.9.0-dev.1 | Ben-Kiki et | https:// |
| | al. (2022) | github.com/ |
| | | tanaylab/ |
| | | metacells#id3 |
| Prism 8 | GraphPad | https:// |
| | | www.graphpad.com/ |
| | | scientific- |
| | | software/prism/ |
| RStudio | RStudio | https:// |
| | | www.rstudio.com/ |
| Smart Denoise | Gurdon | N/A |
| | Institute | |
| Other | | |
| AggreWell400 | STEMCELL | Cat# 34,415 |
| | Technologies | |
| Anti-Adherence Rinsing Solution | STEMCELL | Cat# 07,010 |
| | Technologies | |
| FACSAria III | BD Biosciences | N/A |
| Human cord serum | Cambridge Blood | N/A |
| | and Stem | |
| | Cell Biobank | |
| Knockout serum replacement | Gibco | Cat# 10828010 |
| Leica SP5 | Leica | N/A |
| | Microsystems | |
| Leica SP8 | Leica | N/A |
| | Microsystems | |
| M-MuLV Reverse Transcriptase | New England | Cat# M0253S |
| | Biolabs | |
| Non-adherent 6 well multi-well plate | Greiner Bio-One | Cat# 657,185 |
| 48-well multi-well plate | Greiner Bio-One | Cat# 677,102 |
| Rat serum (CD female) | Charles River | N/A |
| | Laboratories | |
| StepOnePlus ™ Real-Time PCR System | Applied | N/A |
| | Biosystems | |
| TRIzol Reagent | Invitrogen | Cat# 15596026 |

All cell lines were cultured and passaged as previously reported. Briefly, all cell lines were cultured at 37° C., 5% CO₂ and 21% O₂. All ESC lines were cultured on gelatin-coated plates in N2B27 consisting of 50% DMEM/F12 (Gibco 21,331-020), 50% Neurobasal A (Gibco 10,888-022), 1% B27 (Gibco A1895601), 0.5% N2 (made in-house), 0.1 mM beta-mercaptoethanol (Gibco 31,350-010), 1% penicillin/streptomycin (Gibco 15,140-122) and 2 mM Glutamax (Thermo Fisher Scientific 35,050-038), supplemented with 3 μM CHIR99021 (Cambridge Stem Cell Institute), 1 μM PD0325901 (Cambridge Stem Cell Institute) and 10 ng/ml mouse leukemia inhibitory factor (Cambridge Stem Cell Institute). TSCs were cultured on mitotically inactivated mouse embryonic fibroblasts (MEFs, Insight Biotechnology, ASF-1201) in FC (feeder cell) media supplemented with 1 μg mL⁻¹ heparin (Sigma-Aldrich H3149-25KU), 25 ng mL⁻¹ Fgf4 (R&D Systems 7486-F4-025) and 25 ng mL⁻¹ Fgf2 (Cambridge Stem Cell Institute). FC media is a DMEM-based media (Gibco 41,966-029) with 15% heat-inactivated FBS (Gibco 10,270-098), 0.1 mM beta-mercaptoethanol (Gibco 31,350-010), 1% penicillin/streptomycin (Gibco 15,140-122), 2 mM Glutamax (Thermo Fisher Scientific 35,050-038), 1% NEAA (Gibco 11,140-035) and 1 mM sodium pyruvate (Gibco 11,360-039). Cell lines were routinely passaged every 3-4 days by treating with trypsin-EDTA (Gibco 25,300,054) for 4 min, followed by inactivation with double volume of FC media. ESC cell pellet was washed with PBS before resuspending in N2B27 2i/LIF for replating. TSC cell pellet was washed with PBS, resuspended in FC media and added to gelatin-coated well to deplete MEFs for 20 min. TSC cell suspension was then collected and replated onto MEFs. Mycoplasma tests were carried out every two weeks to exclude contamination. Genotyping was not performed to determine the sex of the cell lines.

### Mouse Model and Recovery of Mouse Embryos

CD-1 mice were maintained, adhering to national and international guidelines. Experiments were performed under the regulation of the Animals (Scientific Procedures) Act 1986 Amendment Regulations 2012 and were reviewed by the University of Cambridge Animal Welfare and Ethical Review Body (AWERB). Natural mating was performed with six-week-old CD-1 females and mouse embryos were recovered at embryonic days E6.5 by dissecting from the deciduae in M2 medium, as previously described.

### Plasmids and Transfecion

Cdx2 DNA flanked by attB sites was PCR-amplified from TSC cDNA using Cdx2-attB primers. The sequences of the primers were: Cdx2-attB forward PCR primer (GGGGACAAGTTTGTACAAAAAAGCAGGCTTAATGTACGTGAGCTACCTTCTGGAC) (SEQ ID NO: 1); and Cdx2-attB reverse PCR primer (GGGGACCACTTTGTACAAGAAAGCTGGGTTTCACTGGGTGACAGTGGAGTTTAAAAC) (SEQ ID NO: 2). Using Gateway technology (Thermo Fisher Scientific), it was then cloned into PB-tetO-hygromycin according to the manufacturer's instructions and the resulting plasmid, PB-tetO-hygro-Cdx2 was verified by Sanger sequencing. PB-tetO-hygro-Cdx2 was then transfected into 12,000 CAG-GFP ESCs together with pBAse and rtTA-zeocyin (0.25 μg each) using Lipofectamine 3000 Transfection Reagent (Invitrogen L3000001). Antibiotic selection was performed for 7 days with hygromycin (1:250; Gibco 10,687,010) and zeocyin (1:1000; InvivoGen ant-zn-1), followed by clonal expansion.

RNA Exraction and qRT-PCR

RNA was extracted from cell pellets using either Trizol Reagent (Invitrogen 15,596-026) or RNeasy Mini Kit (Qiagen 74,104). It was subsequently reverse transcribed into cDNA with M-MuLV reverse transcriptase (New England Biolabs M0253S). qRT-PCR was carried out using SYBR Green PCR Master Mix (Applied Biosystems 4,368,708) and StepOnePlus Real-Time PCR System (Applied Biosystems). ΔΔCt method was used to calculate fold change using GAPDH as endogenous control. Sequences of qPCR primers for Cdr2 and Gapdh were taken from published papers while other qPCR primers were designed and validated in this study. The sequences of the qPCR primers were:

Cdx2 forward:
(SEQ ID NO: 3)
AAACCTGTGCGAGTGGATG
Cdx2 reverse:
(SEQ ID NO: 4)
TCTGTGTACACCACCCGGTA
Elf5 forward:
(SEQ ID NO: 5)
ATTTCTACAGTCCGCTGGTGC
Elf5 reverse:
(SEQ ID NO: 6)
ACATCACCGTGAAGACAAGTGG
Gapdh forward:
(SEQ ID NO: 7)
CGTATTGGGCGCCTGGTCAC
Gapdh reverse:
(SEQ ID NO: 8)
ATGATGACCCTTTTGGCTCC
Gata3 forward:
(SEQ ID NO: 9)
TGTCTGCGAACACTGAGCTG
Gata3 reverse:
(SEQ ID NO: 10)
CGATCACCTGAGTAGCAAGGAG.

### Formation of Cell Aggregates and EiTiX-Embryoids

Cell aggregates and EiTiX-embryoids were generated largely following the previously described method with some modifications.

To generate cell aggregates, 1 mg/mL doxycycline (Sigma-Aldrich D9891-5G) was added to iCdx2 ESCs for 6 h. Then, each AggreWell (STEMCELL Technologies 34,415) was treated with 500 μL anti-adherence rinsing solution (STEMCELL Technologies 07,010) and the AggreWell plate was centrifuged at 2000 g for 5 min to remove bubbles in microwells. Anti-adherence rinsing solution was kept in the AggreWell for 20 min. ESCs were then dissociated using the above method (cell lines and culture conditions) and cell density in the cell suspension was determined using a hemocytometer. For cell aggregates, 38,400 6-h induced iCdx2 ESCs, 19,200 uninduced iCdx2 ESCs, and 19,200 TSCs were needed per AggreWell. After determining the volume of cell suspension needed, cell suspension was centrifuged at 1,000 rpm for 4 min before resuspending in 1 mL FC media supplemented with 1 μg mL⁻¹ heparin (Sigma-Aldrich H3149-25KU), 25 ng mL⁻¹ Fgf4 (R&D Systems 7486-F4-025) and 7.5 nM Y27632 (STEMCELL Technologies 72,304). The anti-adherence rinsing solution was aspirated from AggreWell and each AggreWell was washed twice with 1 mL PBS before adding 500 μL FC media with heparin and Fgf4 (FCF4H media) per well. Cell suspension was then added drop-wise onto AggreWell and the plate was centrifuged at 100 g for 3 min. In each AggreWell, 1 mL of media was replaced with 1 mL fresh FCF4H media on Day 1 and Day 2.

To generate EiTiX-embryoids, 1 mg/mL doxycycline (Sigma-Aldrich D9891-5G) was added to both iCdx2 ESCs and iGata4 ESCs for 6 h. AggreWell was treated same as the above for generating cell aggregates. 6-h induced iCdx2 ESCs, 6-h induced iGata4 ESCs and WT ESCs were dissociated and cell density was determined. For each AggreWell, 38,400 6-h induced iCdx2 ESCs, 6,000 6-h induced iGata4 ESCs, and 6,000 WT ESCs were needed and the cell suspensions were pooled together to be centrifuged at 1,000 rpm for 4 min. The resulting cell pellet was resuspended in 1 mL FC media supplemented with 1 μg mL⁻¹ heparin, 25 ng mL⁻¹ Fgf4 and 10 nM ROCK inhibitor. The anti-adherence rinsing solution was aspirated from AggreWell and each AggreWell was washed twice with 1 mL PBS before adding 500 μL FC media with heparin and Fgf4 per well. Cell suspension was then added drop-wise onto AggreWell and the plate was centrifuged at 100 g for 3 min. On Day 1, 1 mL of media was replaced with 1 mL fresh FC media and media change was repeated once to ensure removal of Y27632. On Day 2, 1 mL of media was replaced with 1 mL fresh FC media. On Day 3, 1.2 mL media was removed and 1.5 mL peri-implantation media (20% FBS) was added per well. Peri-implantation media (also known as IVC media) contained 80% advanced DMEM/F12 (Gibco 12,634-010) supplemented with 20% FBS, 2 mM GlutaMax, 1% penicillin-streptomycin, 1× insulin-transferrin-selenium-ethanolamine (ThermoFisher Scientific 51,500-056), 8 nM β-estradiol (Sigma-Aldrich E8875-1G), 200 ng/mL progesterone (Sigma-Aldrich P8783-1G) and 25 mM N-acetyl-L-cysteine (Sigma-Aldrich A7250). On Day 4, structures were selected under dissection microscope (see below for inclusion criteria of EiTiX embryos) and up to 50 structures were transferred to each well of non-adherent 6 well multi-well plate (Greiner Bio-One 657,185), which contained 5 mL peri-implantation media (with 30% FBS). On Day 5, structures were transferred to peri-implantation media (with 30% knockout serum replacement, Gibco 10,828,010). Peri-implantation media was equilibrated in the incubator for 20 min in advance.

### Culture of EiTiX-Embryoids in Post-Implantation Culture Medium

Post-implantation culture medium was prepared as previously described. It comprises 25% DMEM (Gibco 11,054 (using HCO₃ buffer) or Gibco 11,880 (using HEPES)) with Ix Glutamax (Gibco 35,050,061), 100 units/mL penicillin/streptomycin (ThermoFisher 15,140,122) and 11 mM HEPES (Gibco 15,630,056 added to DMEM-Gibco 11,880), plus 50% CD female rat serum (Charles River Laboratories) and 25% human cord serum (Cambridge Blood and Stem Cell Biobank). Rat serum and human cord serum were thawed at room temperature and heat-inactivated for 30 min at 56° C. After preparation, post-implantation culture medium was filter-sterilized and equilibrated at 37° C. for 1 h. Each of the selected Day 5 EiTiX-embryoids was transferred to one well of a 48-well multi-well plate for suspension culture (Greiner Bio-One 677,102), with 250 μL post-implantation culture medium per well. On Day 6, 100 μL medium was removed and 250 μL fresh post-implantation culture medium was added per well. On Day 7, EiTiX-embryoids were transferred to a rotating bottle culture chamber apparatus. Up to 3 EiTiX-embryoids were cultured in the same rotating bottle that contained 2 mL post-implantation medium supplemented with 3.0 mg/mL of D-Glucose (Sigma G8644).

### Immunofluorescence

Samples were fixed with 4% paraformaldehyde for 20 min at room temperature and then washed three times with PBST (0.1% Tween 20 in PBS). Samples were permeabilized in 0.1 M glycine and 0.3% Triton X-100 in PBS for 30 min at room temperature. After washing with PBST for three times, primary antibodies diluted in blocking buffer (10% FBS and 0.1% Tween 20 in PBS) were added and the samples incubated overnight at 4° C. Primary antibodies were removed the following day, and samples were washed three times with PBST, before adding secondary antibodies and DAPI. After incubating overnight at 4° C., samples were washed three times with PBST and mounted in a glass-bottom dish for imaging.

### Whole Mount In Situ Hybridization

Whole mount in situ hybridization was performed following a published protocol. Briefly, EiTiX-embryoids were fixed with 4% paraformaldehyde at 4° C. overnight and were dehydrated with methanol series. After rehydrating with inverted methanol series, Day 5 and Day 6 EiTiX-embryoids were treated with proteinase K for 3 and 4 min respectively and were subsequently washed with PBST and refixed in 4% paraformaldehyde/0.2% glutaraldehyde. Then, EiTiX-embryoids were washed in 50 and 100% hybridization buffer and denatured Bmp4 probe were added for overnight hybridization at 70° C. Probe was removed by washing in hybridization buffer, SSC, MA buffer, PBS and PBST. Next, EiTiX-embryoids were incubated in antibody buffer and preblocked antidigoxigenin antibodies were added and incubated overnight at 4° C. EiTiX-embryoids were then washed in PBST/0.1% BSA, PBST, and API buffer. API buffer with BM purple was added for color development and the reaction was stopped by washing in STOP solution.

### Dissociation of EiTrX-Embryoids for MARS-Seq

Individual Day 6 EiTiX-embryoids were dissected into four pieces with needles in PBS, which were then dissociated with 70 μL TrypLE Express Enzyme (Gibco 12,604,021) at 37° C. for 15 min, pipetting up and down every 5 min. Dissociation was stopped by adding 500 μL FC media supplemented with Y27632 (1:2,000) and DAPI (1:2,000). Day 8 samples were dissociated with 200 μL TrypLE Express Enzyme and 800 μL media was added to stop the dissociation. The cell suspension was subsequently filtered through a 40 μm cell strainer (Merck CLS431750) and further diluted with 2 mL FC media. It was then sorted by FACSAria III (BD Biosciences) using index sorting into 384-well plates.

### MARS-Seq Library Preparation

Single-cell cDNA libraries were prepared as previously described following the MARS-seq protocol. MARS-seq libraries were processed using NextSeq 500 or NovaSeq 6000. The output reads were processed following the MARS-seq2.0 protocol with the same specifications as previously reported, using STAR aligner for sequence alignment. Here, 8832 wells were processed. To analyze Day 8 EiTiX embryoids, FACS index sorting was used to record fluorescence per cell in addition to structure identity per well. GFP positive cells were distinguished using the green channel bimodal distribution.

### Inclusion Criteria of EiTiX-Embryoids

All EiTiX-embryoids were collected from AggreWell on Day 4 and their morphologies were examined under a dissection microscope. Structures were selected for analyses or further culture if 1) there were two distinct cellular compartments enclosed by a thin outer cell layer, and 2) there was a clear epithelialized ES compartment with a central lumen. For Day 5 and Day 6 EiTiX-embryoids, structures that were elongated and had a thick epithelial cell layer in the ES compartment that resembled the EPI in natural mouse embryos were selected. On Day 7, EiTiX-embryoids that had developed headfolds structure within a yolk sac-like membrane were selected.

### Atlas Projection and Cell Type Annotation of EiTiX-Embryoids

To identify marker genes for metacell construction, all the genes displaying a minimal variance over mean (T_vm=0.1) and coverage threshold (Tₜₒₜ=50, Tₜₒₚ₃=3) were selected. These genes were clustered into 137 clusters based on their gene-gene correlation overall the UMI mat. Gene-cluster enriched with stress- and cell-cycle-related genes were manually removed (n=625) leaving 807 feature genes. The final metacell object (Kₘ=100, minimal metacell size=30 cells) contained 60 metacells comprised of 7076 cells (2184 from Day 6 EiTiX structures and 4892 from Day 8 EiTiX-embryoids) with a 5362 median UMIs per cell. Metacells were annotated with cell types by projection on the gastrulation wild-type (WT) atlas, as previously reported.

### Natural Embryo Matching

For each EiTiX-embryoid, a best-matching natural embryo was inferred based on the similarity (Euclidean distance) between their cell state compositions. Only natural embryos with at least 161 embryonic cells and 29 ExE cells were included (threshold fits the calculated median number of ExE cells per EiTiX-embryoid). For each EiTiX-embryoid, the three closest natural embryos in the matching natural cohort were included, counting each natural embryo only once. Natural embryos were temporally ordered as previously reported.

Mean Differential Expression Among Transcriptional States from EiTiX-Embryoids

For each EiTiX Day, bulk (average) gene expression profiles per cell type were computed and compared with the corresponding natural embryo expression profiles from the matching natural embryos (log 2 absolute expression). For each cell type, the number of included natural embryo cells was down sampled to the corresponding number of cells from EiTiX-embryoids compared. Cell-cycle and stress-related genes were not included in that comparison. Highlighted cell-type-specific genes for each included cell type were defined as being on average at least 2-fold enriched in the metacells from this cell type relative to the global average among all natural embryo Metacells and additional known cell type markers were added.

### Image Acquisition, Processing and Analysis

Leica SP5 and SP8 confocal microscopes (Leica Microsystems) with either a 40× oil objective or a 25× water objective were used to acquire immunofluorescence images. A 405 nm diode laser, 488 nm argon laser, 543 nm HeNe laser and 633 nm HeNe laser (Alexa Fluor 647) were used to excite the fluorophores. Fiji and Smart Denoise (Gurdon Institute) were used for image processing and analysis.

### Quantification of the Extent of Anterior Visceral Endoderm Anterior Localization

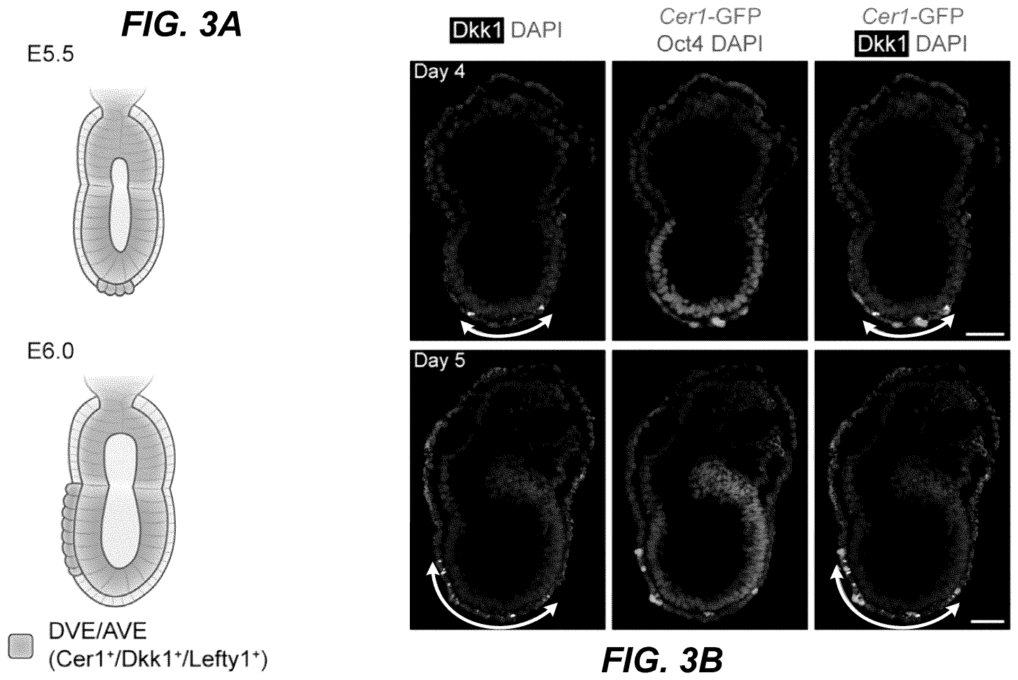

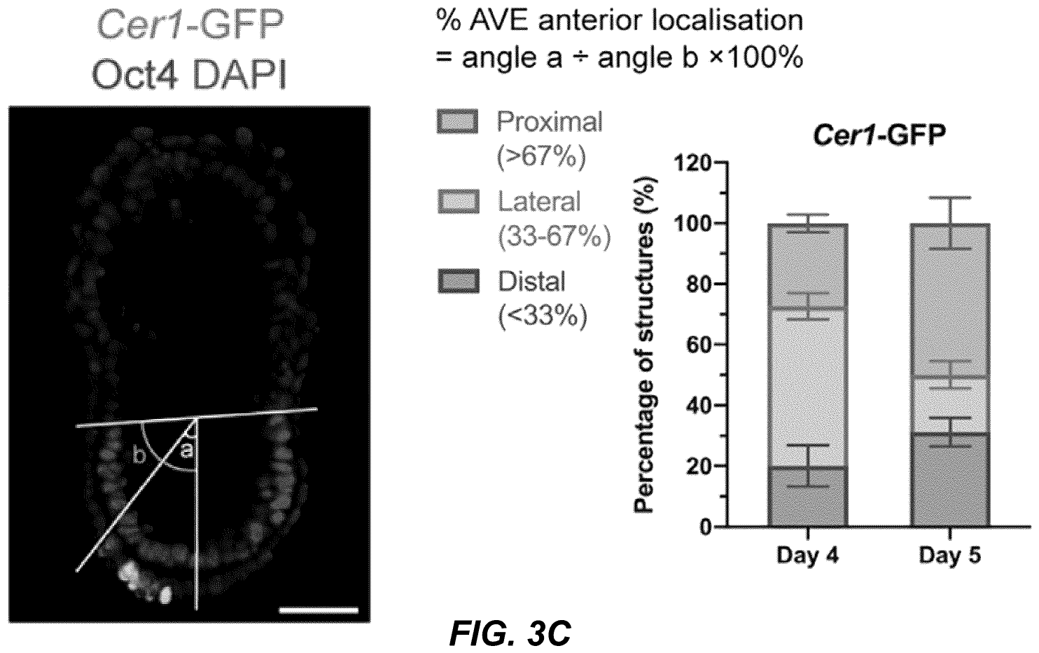

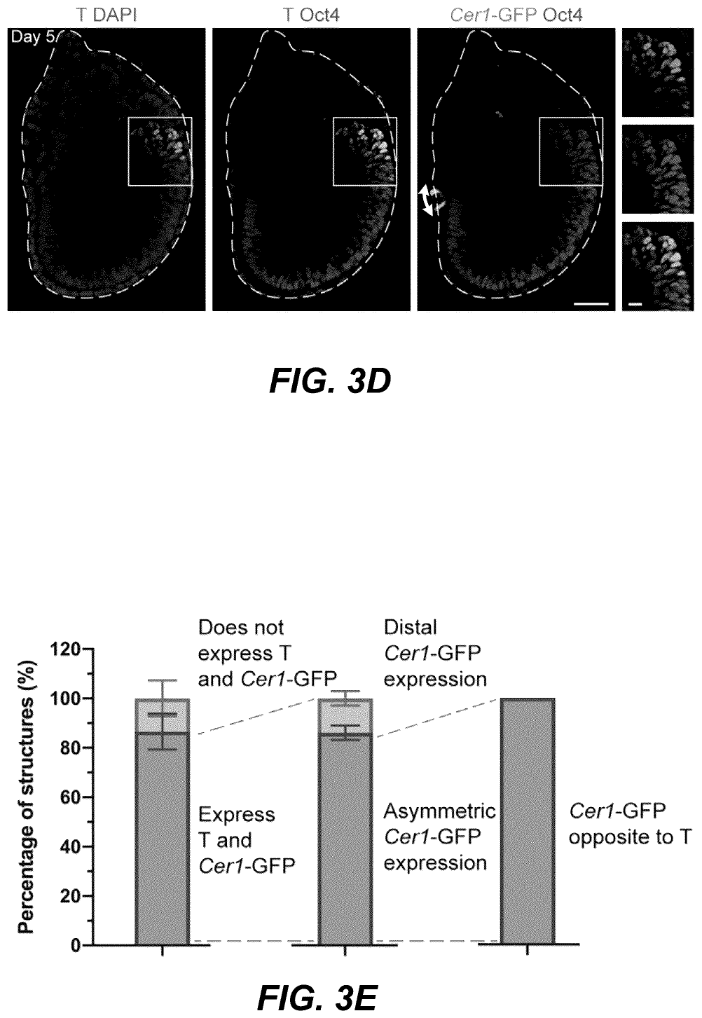

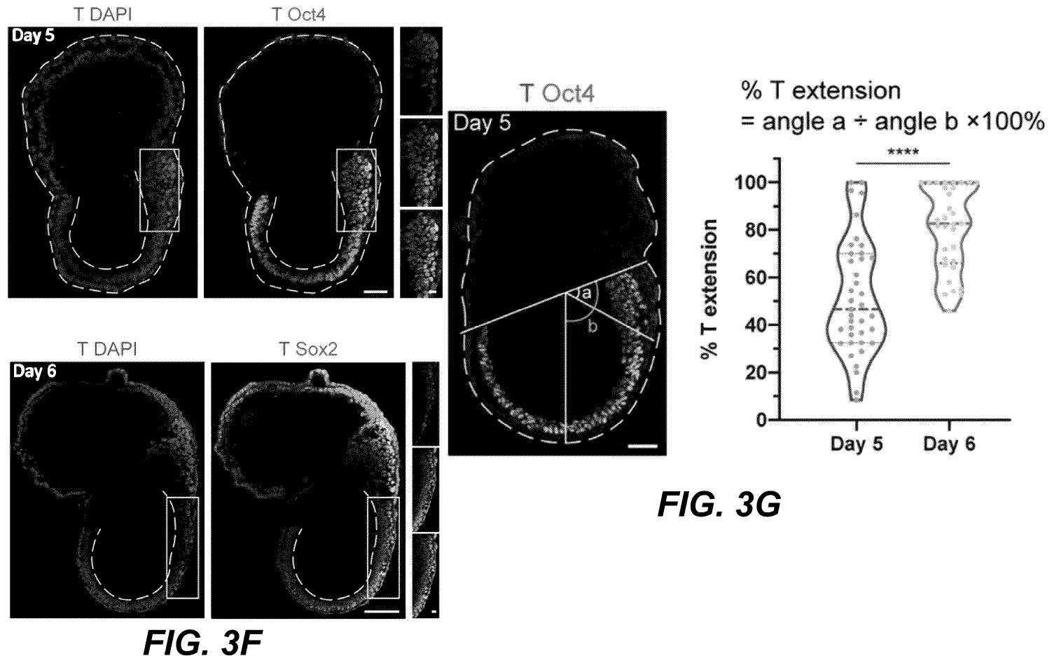

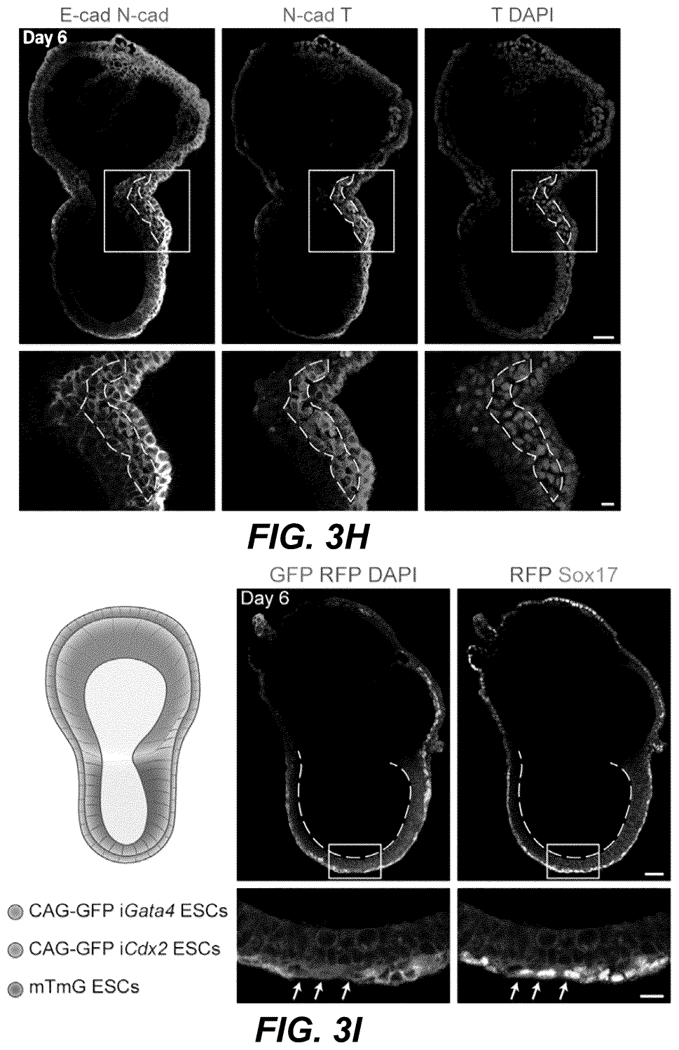

The angle between the distal tip and the most anterior cell expressing AVE marker was termed angle a while the angle between the distal tip and the boundary of Oct4-positive domain was termed angle b (FIG. 3C). Percentage of AVE migration was obtained by dividing angle a by angle b and multiplying by 100%. It was then classified as proximal (>67%), lateral (33%-67%) and distal (<33%).

### Quantification of the Extent of Brachyury (7) Extension

The angle between the posterior boundary of Oct4-positive domain and the most anterior T-positive cell was termed angle a while the angle between the posterior boundary of Oct4-positive domain and the distal tip The percentage of T extension was obtained by dividing angle a by angle b (FIG. 3G) and multiplying by 100%.

### Statistics

Statistical analyses were performed using GraphPad Prism 8 and quantitative data were presented as mean f SEM or as violin plots with median and quartiles. Student's t test was used to determine statistical significance between two samples while one-way ANOVA followed by Bonferroni's multiple comparisons test was used to determine statistical significance between more than two groups. Sample size and number of experimental replicates were indicated in figure legends.

### Example 1

### Cdx2-Induced ESCs Self-Assemble with Gata4-Induced ESCs and ESCs into Post-Implantation-Like Mouse Embryoids

This example investigates the possibility of replacing TSCs with Cdx2-expressing ESCs in generating synthetic embryo structures.

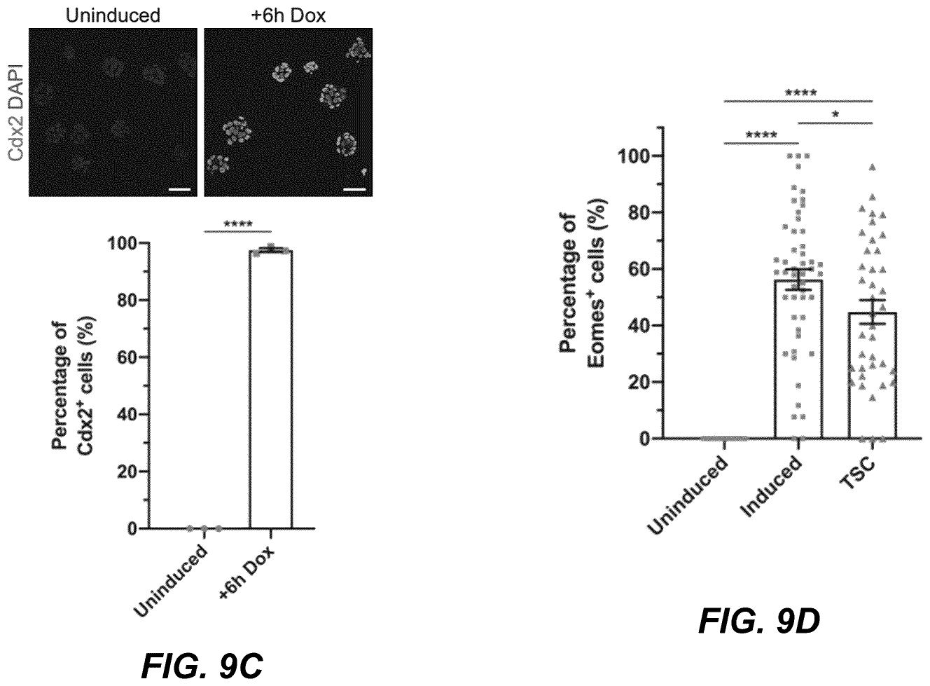

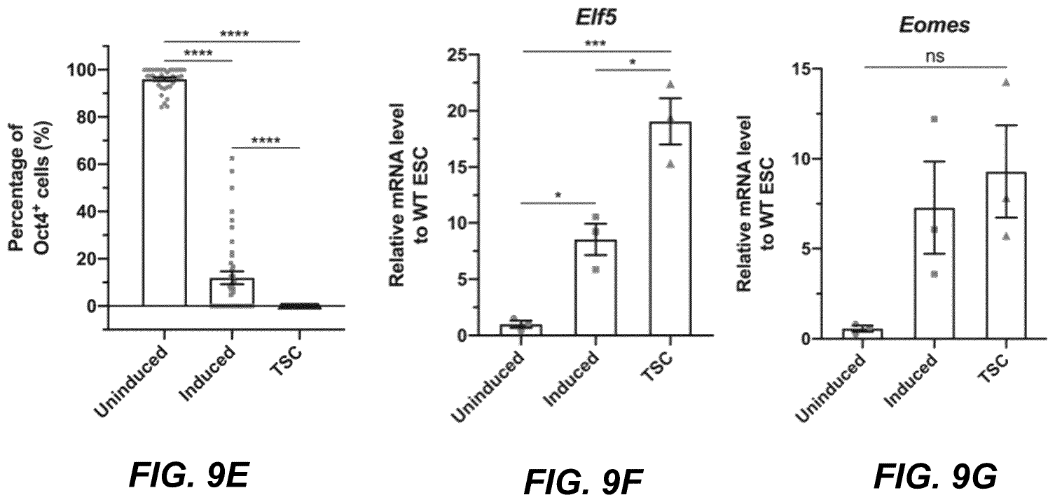

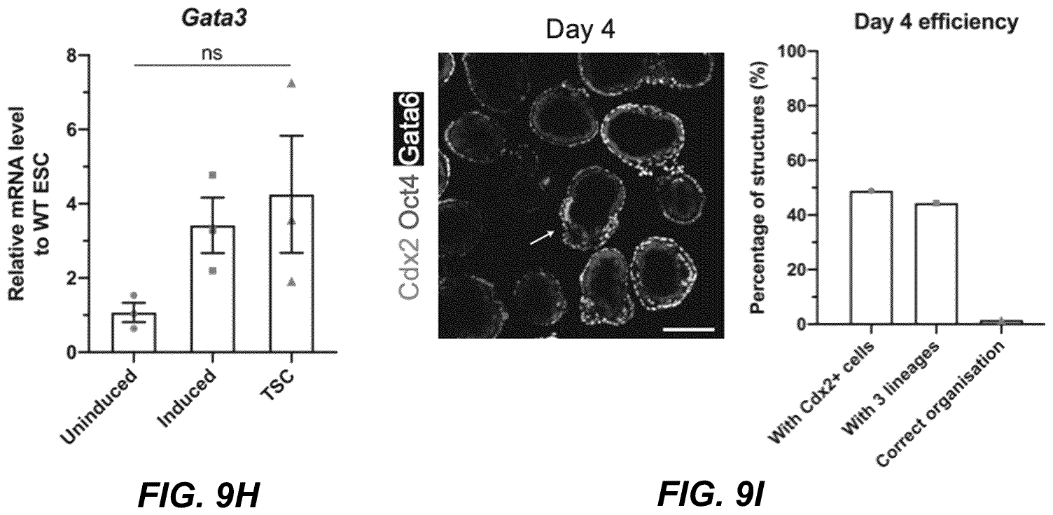

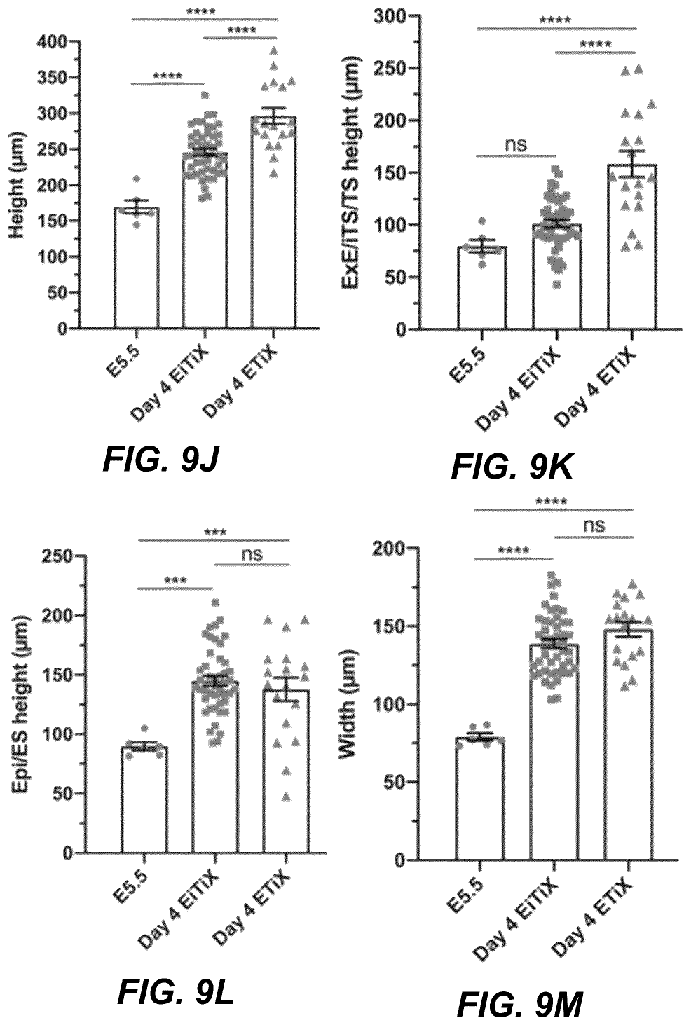

Cdx2 is a key transcription factor driving TE development and its overexpression leads ESCs to transdifferentiate into TSC-like cells. To determine whether Cdx2-expressing ESCs could replace TSCs in generating ETiX-embryoids (formerly termed iETX-embryoids), a transgenic ESC line carrying a doxycycline (Dox)-inducible Cdx2 gene was generated. The resulting clones of iCdx2-ESCs showed a 100- to 200-fold increase in Cdx2 mRNA expression after 6 h of Dox induction (FIG. 9A). From the four clones tested, the clone with the highest level of Cdx2 overexpression was selected for subsequent experiments. This clone showed a substantial upregulation of both Cdx2 mRNA (FIG. 9B) and protein, as detected by qRT-PCR and immunofluorescence, respectively, after 6 h of induction (FIG. 9C). To assess the long-term effect of Cdx2 overexpression on cell fate, three different types of cell aggregates: induced iCdx2 ESCs, uninduced iCdx2 ESCs, or TSCs (FIG. 2A) were compared. After 3 days, a significant upregulation of the TSC marker Eomes and downregulation of the ESC marker Oct4 were observed in the aggregates of induced iCdx2 ESCs (FIG. 2B, FIG. 2C, FIG. 9D and FIG. 9E). Transcripts of the TSC markers Elf5, Eomes, and Gata3 were also upregulated in the induced iCdx2 ESC aggregates (FIG. 9F-FIG. 9H). Together, these findings suggest that upon Cdx2 overexpression, iCdx2 ESCs lose their ESC identity and acquire TSC-like cell fate.

Then, whether induced iCdx2 ESCs could replace TSCs in generating embryoids when aggregated with wild-type (WT) ESCs and Gata4 induced ESCs was investigated. To this end, a previously described protocol was adapted by inducing expression of Cdx2 and Gata4 by treating both iCdx2- and iGata4-ESC lines for 6 h with Dox before combining them with WT ESCs in AggreWell plates (FIG. 2D). Over the course of 4 days, drastic morphological changes of the resulting cell aggregates were observed such that by Day 4 structures that resembled post-implantation embryos, which naturally comprise EPI and ExE compartments surrounded by visceral endoderm (VE) (FIG. 2E), could be observed. The random nature of the interactions of the three cell types results in a variety of structures (FIG. 9I). Thus, the efficiency of correct structure formation was optimized by adding Fgf4 and heparin during the first 24 h after plating and by doubling the number of iCdx2 ESCs seeded from 16 to 32 per microwell (FIG. 2D and FIG. 2F). The efficiency of correct structure formation on Day 4 was 15.5%, which is slightly lower than that of the ETiX-embryoid system, reflecting differences between using iCdx2 ESCs and TSCs (FIG. 2F). By setting up several AggreWells that each contain 1,200 microwells at the start of the experiment, around 350 well-developed embryo-like structures per experiment would be routinely obtained on Day 4 (FIG. 2G), which exceeded the capacity to transfer all of them to post-implantation culture conditions due to the costs of serum for culture media.

When the expression of the constitutive membrane GFP marker of iCdx2 ESCs was examined, it was found that iCdx2 ESCs had given rise to the Cdx2-positive cells that correctly localized within the ExE-like compartment (FIG. 2H). The expression of other ExE markers including Ap2γ and Eomes in the putative ExE compartment was also detected, suggesting that downstream TSC markers were also upregulated after Cdx2 overexpression (FIG. 2I). Finally, the dimensions of EiTiX-embryoids, E5.5 mouse embryos, and Day 4 ETiX-embryoids were compared, revealing that EiTiX-embryoids were most similar to E5.5 embryos (FIG. 9J-FIG. 9M). Together, the results have shown that iCdx2 ESCs can replace TSCs to generate post-implantation embryo-like structures expressing canonical lineage markers. Since TSCs were replaced by iCdx2 ESCs, the structures were termed “EiTiX-embryoids.”

### Example 2

### EiTiX-Embryoids Establish an Anterior-Posterior Axis and Undergo Gastrulation

This example is carried out to investigate whether EiTiX-embryoids could recapitulate key events of post-implantation development.

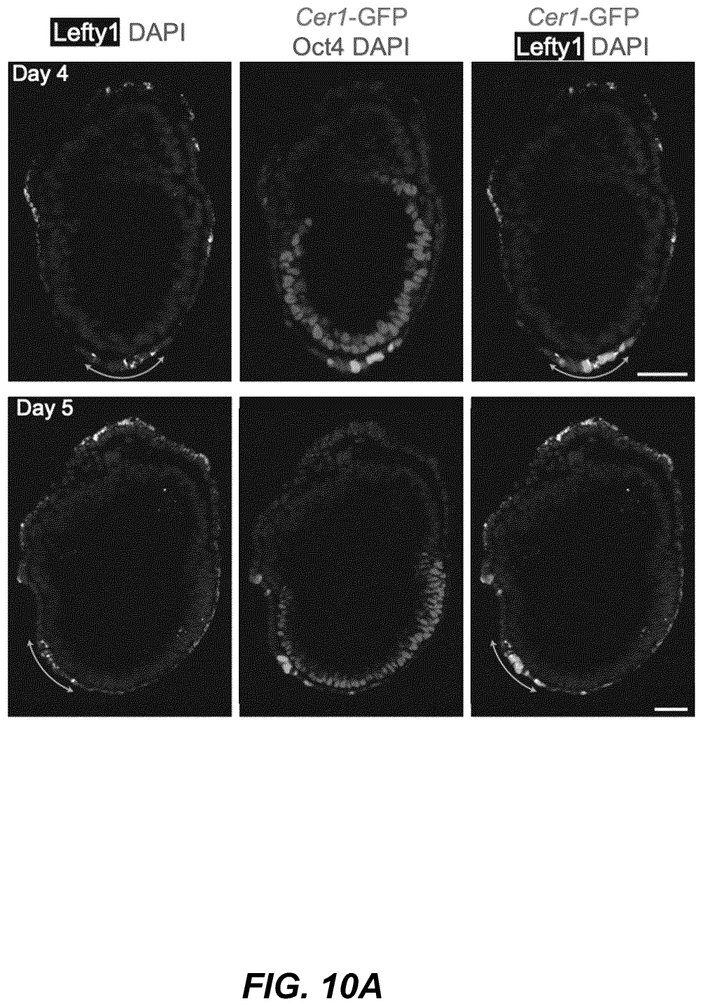

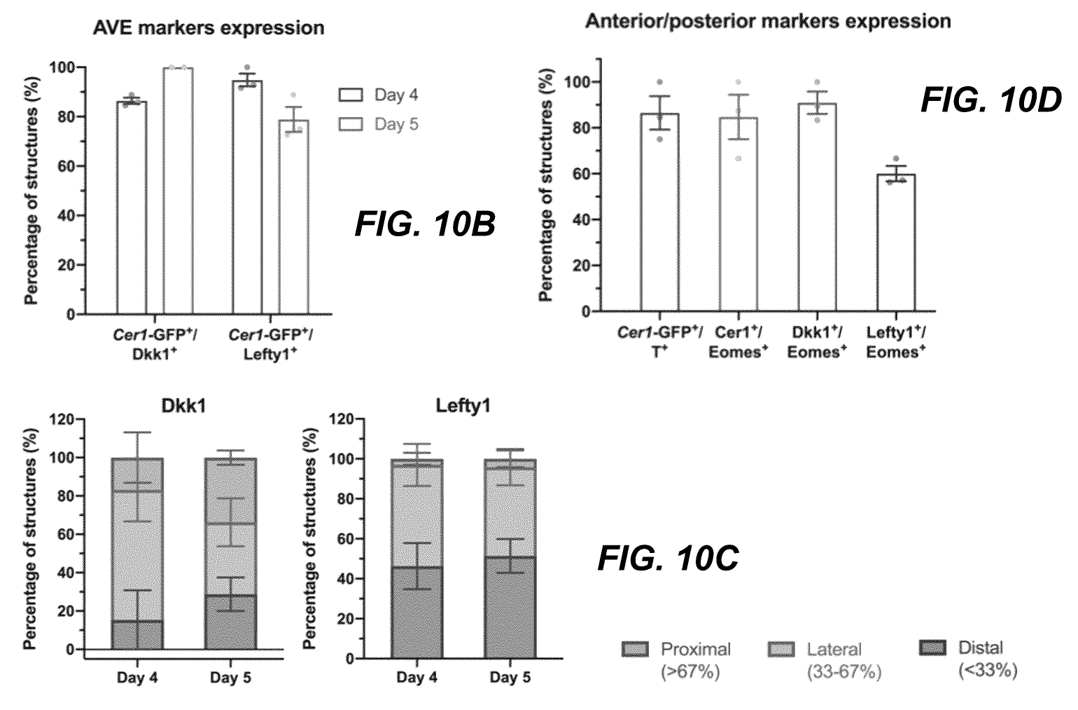

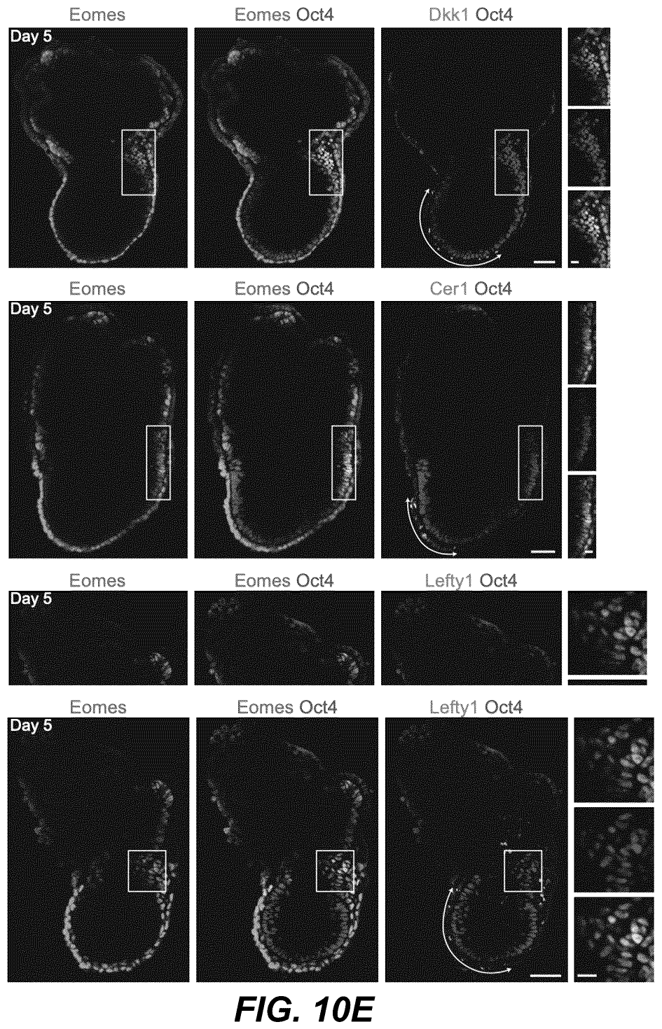

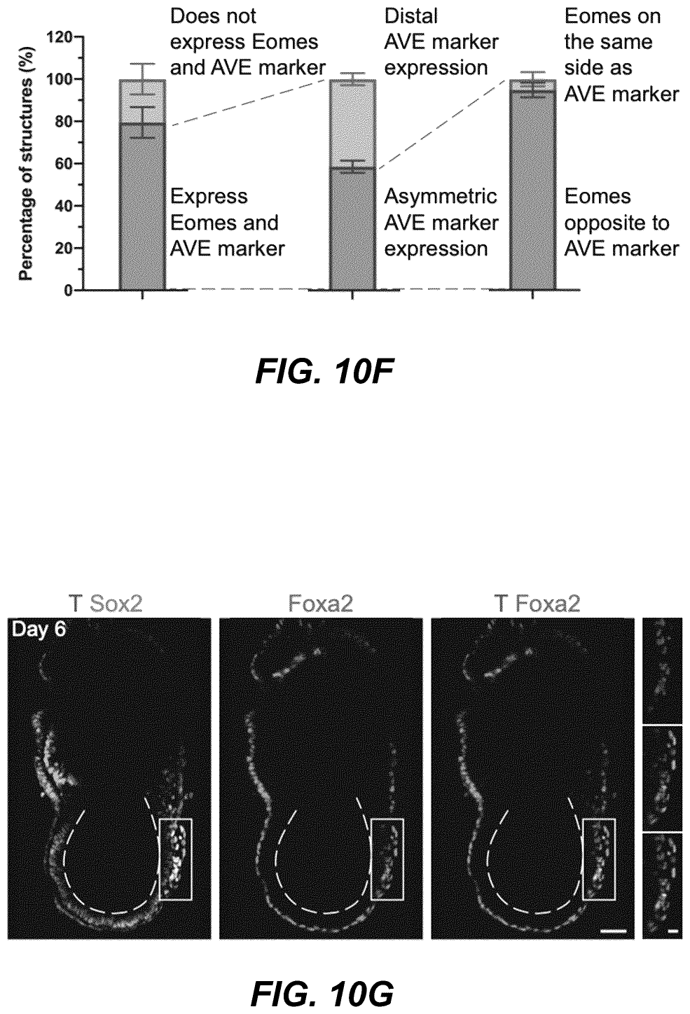

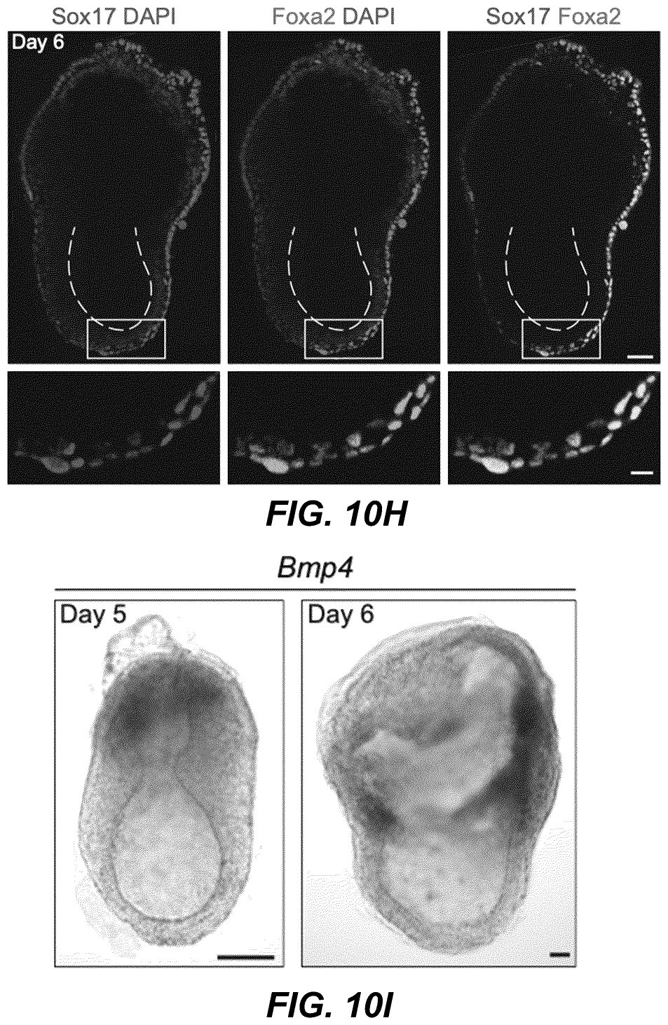

First, whether the critical anterior signaling center can be formed was determined, which breaks mouse embryo symmetry and establishes the anterior-posterior axis. This center first appears as the distal visceral endoderm (DVE) at the distal tip of the egg cylinder before migrating to the anterior side of the egg cylinder to become the anterior visceral endoderm (AVE), which is characterized by the expression of Cer1, Lefty1, and Dkk1 (FIG. 3A). To this end, EiTiX-embryoids were formed using iGata4 ESCs with a Cer1-GFP reporter and the co-expression of GFP with Dkk1 or Lefty1 was observed in EiTiX-embryoids on Day 4 and Day 5 (FIG. 3, FIG. 10A and FIG. 10B). To follow the development of the Cer1-GFP-positive domain, the extent of AVE anterior migration was determined. Moreover, the measurements were binned into three groups: “proximal,” >67% migration; “lateral,” 33%-67% migration, and “distal,”<33% migration. Anterior migration of the AVE was evident from the higher proportion of Day 5 EiTiX-embryoids with proximal Cer1-GFP and Dkk1 expression lower than that of Day 4 EiTiX-embryoids, while the distribution of Lefty1-positive domain remained similar (FIG. 3C and FIG. 10C).

In the natural post-implantation embryo, the AVE is critical to restrict primitive streak formation to the posterior EPI through the secretion of Nodal and Wnt inhibitors. Therefore, additional experiments were performed to determine whether these events could be recapitulated in EiTiX-embryoids and the expression of Brachyury (T), a primitive streak marker was analyzed, in relation to the Cer1-GFP domain on Day 5. It was found that 86.7% of Day 5 EiTiX-embryoids expressed Cer1-GFP and T, of which 86% showed opposed Cer1-GFP and T expression (FIG. 3D and FIG. 3E). Similarly, 94.7% of structures with asymmetric AVE expression of Cer1, Dkk1, or Lefty1 showed expression of the primitive streak marker Eomes on the opposite side (FIG. 10D-FIG. 10F). Thus, EiTiX-embryoids correctly established both the AVE and primitive streak, recapitulating anterior-posterior patterning as in natural post-implantation embryos.

After establishment of the anterior-posterior axis and onset of gastrulation in the posterior EPI, the primitive streak extends to the distal end of the egg cylinder. Accordingly, as EiTiX-embryoids developed, T- and Oct4-positive cells could be detected at the posterior end of the EPI-like compartment on Day 5 that had extended to the distal-most part of the egg cylinder on Day 6 (FIG. 3F). To quantify the percentile extension of this T-positive domain, the angle between the posterior boundary of the Oct4-positive domain and the most anterior T-positive cells (FIG. 3G, angle a) as well as the angle subtended by the Oct4-positive domain boundary and the distal tip (angle b) were measured, where the percentage of angle a divided by angle b indicates the percentile extension. This showed that the degree of extension approached its fullest extent at Day 6 (FIG. 3G). As cells egress from the EPI to form the primitive streak, they undergo epithelial-to-mesenchymal transition, downregulating E-cadherin and upregulating N-cadherin. A T-positive domain that had robust N-cadherin expression was observed. However, unlike surrounding cells, cells in the T-positive domain did not express E-cadherin in 66.7% of EiTiX-embryoids on Day 6 (FIG. 3H).

As development progresses, the primitive streak undergoes further specification to produce a range of cell types, including axial mesendoderm and definitive endoderm. In Day 6 EiTiX-embryoids, the presence of T- and Foxa2-positive cells identifying axial mesendoderm (93.8%), as well as Foxa2- and Sox17-positive cells identifying definitive endoderm (88.2%) could be detect (FIG. 10G and FIG. 10H). In the natural mouse embryo, the EPI-derived definitive endoderm gradually displaces and intercalates with the VE, which covers the egg cylinder. To visualize whether such endoderm intercalation takes place in EiTiX-embryoids, ESCs expressing membrane tdTomato (mTmG ESCs) was used to generate the EPI-like compartment and iGata4 ESCs expressing membrane GFP (CAG-GFP iGata4 ESCs) was used to generate the VE-like layer in the embryoids. A discontinuous GFP-positive cell layer interspersed with RFP-positive cells was observed (FIG. 3I, 66.7%). These cells also expressed Sox17, which is a critical factor for endoderm specification and for the egression of definitive endoderm cells into the VE. Thus, the intercalation of definitive endoderm into the VE was recapitulated in the EiTiX-embryoids.

This example also investigates whether the ExE-like compartment in EiTiX-embryoid could recapitulate functions of ExE in natural mouse embryos. Bmp4 is a crucial signaling molecule expressed specifically in the ExE, which is important for patterning the embryos through cross-talk with embryonic tissues and specifying mesoderm and primordial germ cells (PGCs). Bmp4 expression was assessed by in situ hybridization and robust Bmp4 expression could be detected in the ExE-like compartment in both Day 5 and Day 6 EiTiX-embryoids (FIG. 10I). Notably, a stronger Bmp4 signal was observed in the posterior ExE-like compartment, in line with the natural mouse embryo where Bmp4 expression is gradually restricted to the posterior. Since Bmp4 is necessary for PGC specification, whether PGC progenitors were present in EiTiX-embryoids was also looked into, but PGC progenitors could not be reliably detected in Day 6 EiTiX-embryoids, which might reflect insufficient Bmp signaling.

### Example 3

### Day 6 EiTiX-Embryoids Capture Major Cell Types of Gastrulation

This example is performed to analyze the overall cell type composition of these gastrulating EiTiX-embryoids in comparison to natural embryos, following the findings in Example 2 that Day 6 EiTiX-embryoids could capture numerous processes of gastrulation.

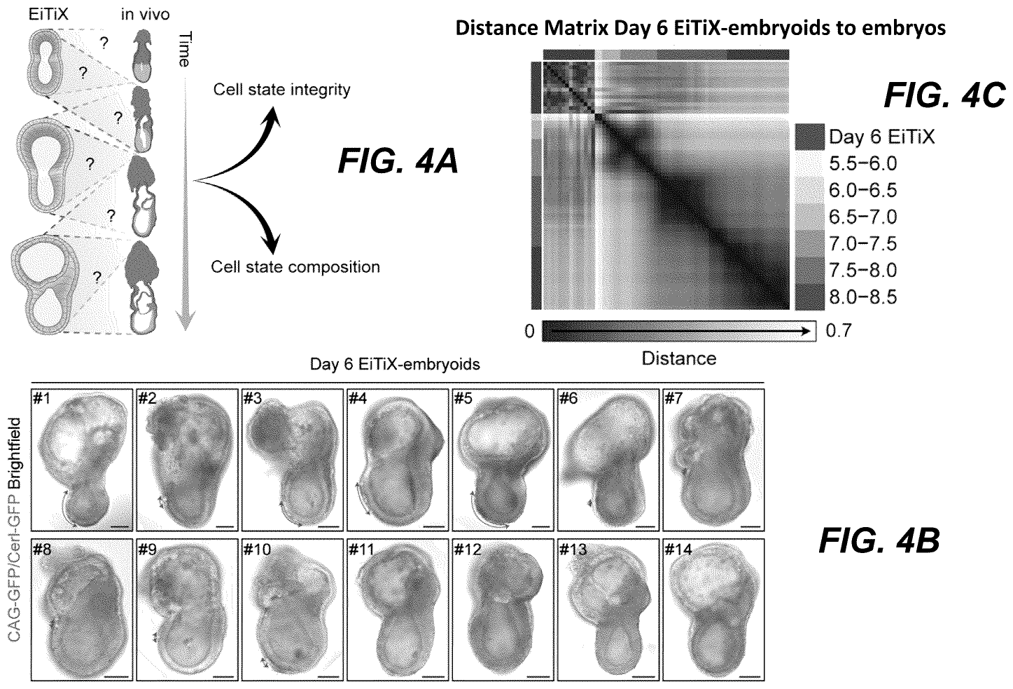

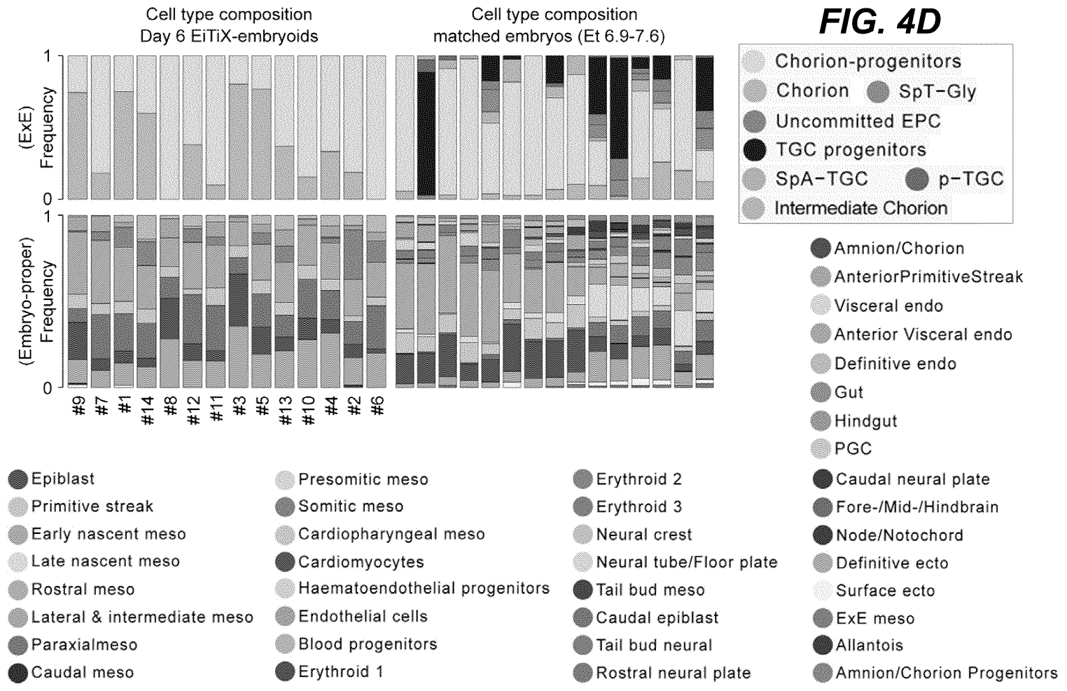

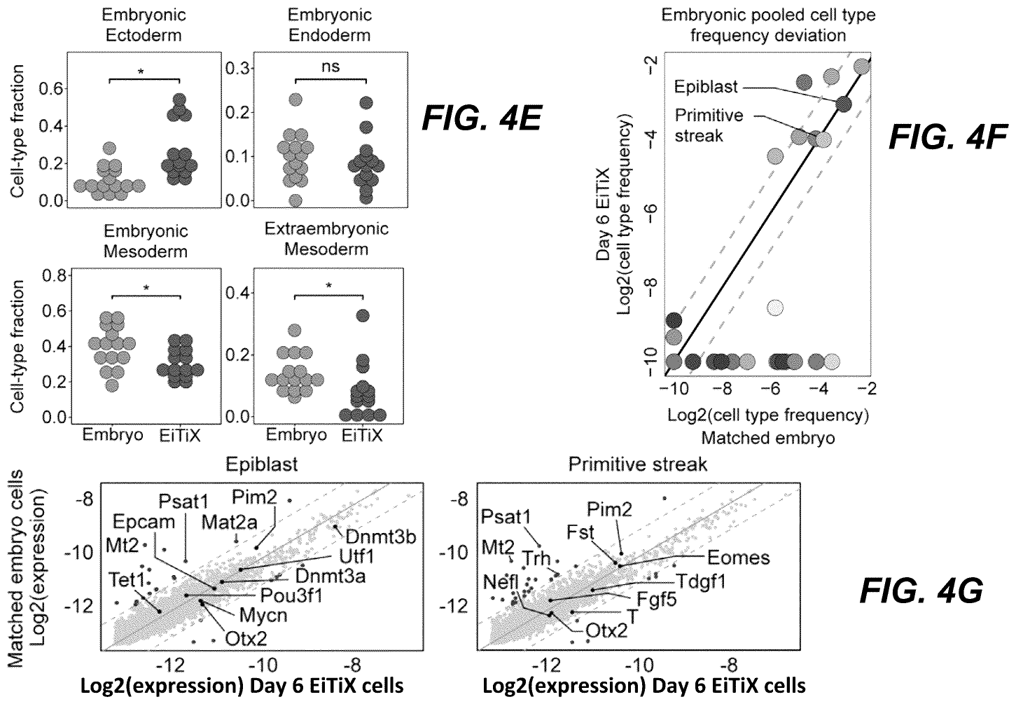

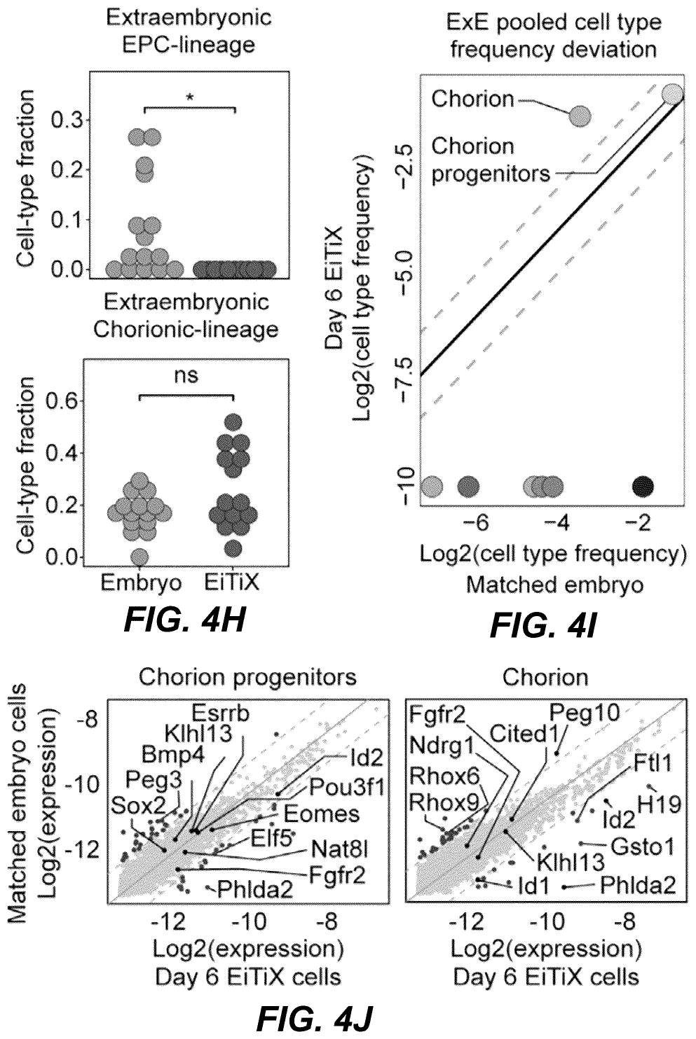

A recently established time-resolved model of mouse gastrulation consisting of ˜68,000 single cells derived from 287 individually processed embryos spanning egg cylinder stage to early somitogenesis (FIG. 4A) was utilized, which (1) enables a quantitative evaluation of transcriptional states; and (2) describes the natural flux of embryonic and extraembryonic lineage differentiation, thus allowing analysis of cell state composition within individual structures. Day 6 EiTiX-embryoids were generated by combining CAG-GFP, unlabeled WT ESCs, and iGata4 ESCs carrying the Cer1-GFP reporter. GFP signals confirmed the appearance of the ExE-like compartment and the AVE-like domain in EiTiX-embryoids (FIG. 4B). Next, single-cell RNA sequencing (scRNA-seq) was performed on 14 individual EiTiX-embryoids using MARS-seq by index-sorting into barcoded 384-well plate as previously reported.

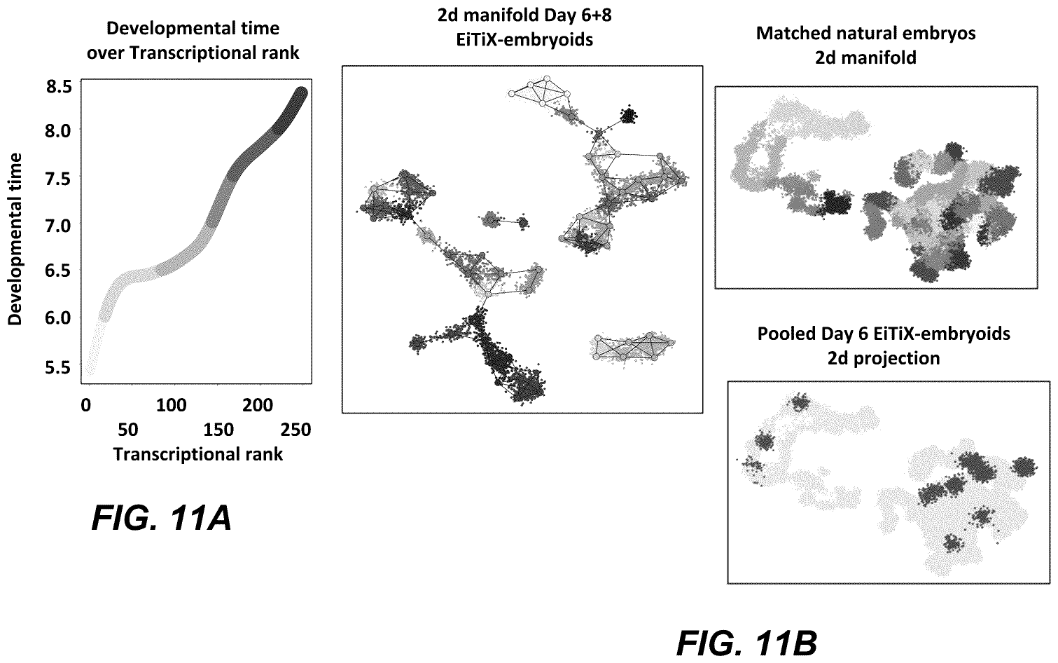

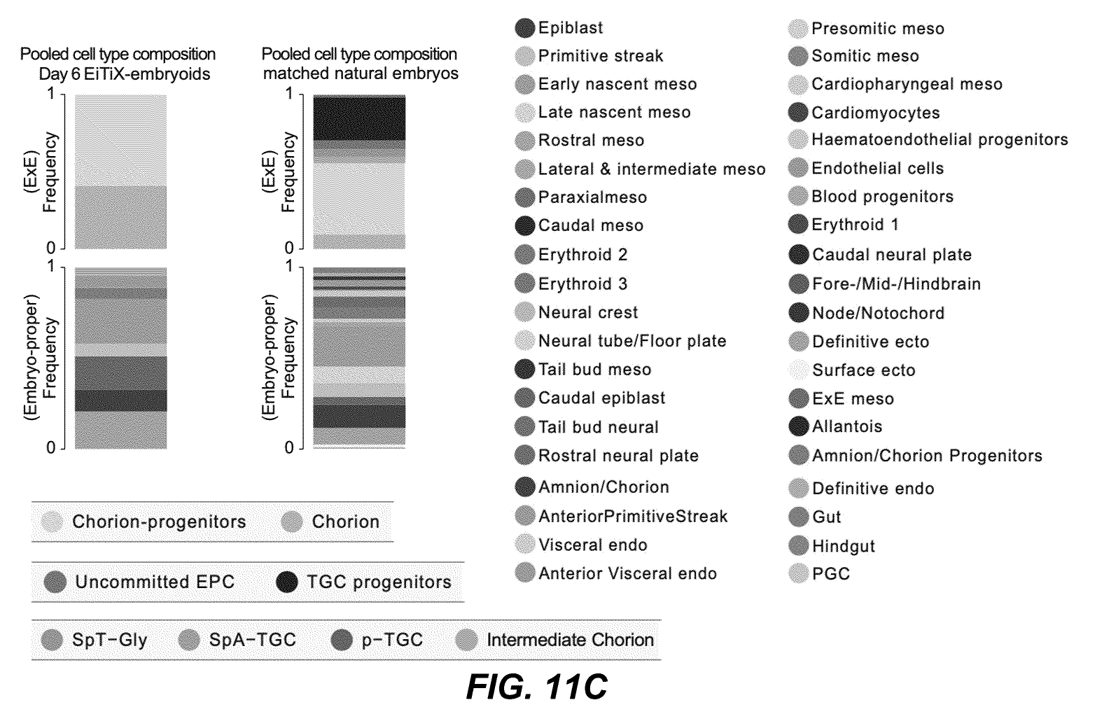

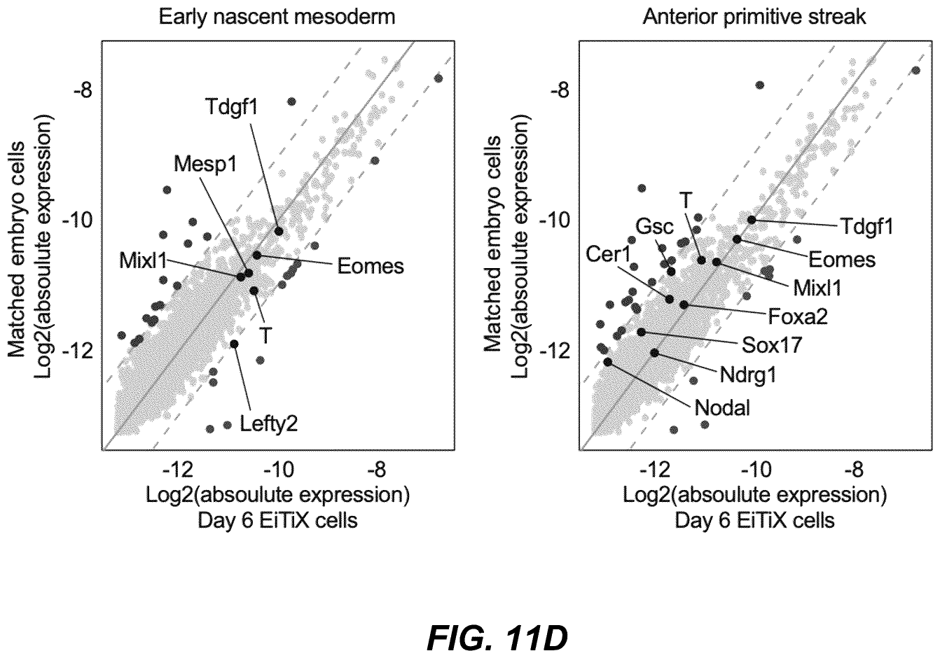

A strategy for ranking embryos by K-nn similarities among their single-cell profiles identified high similarity between individual Day 6 EiTiX-embryoids. In agreement with the morphological assessment of these embryoids, their overall transcriptional ranking was found to be most similar to E6.5-7.5 gastrulation stages (FIG. 4C and FIG. 11A). Next, a transcriptional manifold of EiTiX-embryoids was constructed and annotated. Remarkably, robust mapping to unmodified embryonic and extraembryonic cell states were found and non-coherent transcriptional programs were not detected in the metacells of Day 6 EiTiX-embryoids, suggesting that they conserve the transcriptional programs of the corresponding cell states in natural embryos (FIG. 11B). The study focused first on embryonic cell state compositions. A high degree of similarity was observed among the 14 Day 6 EiTiX-embryoids, despite variable morphologies (FIG. 4D). However, when compared to natural embryos from corresponding time bins, deviations from the natural program were found. First, some of the lineages were not in synchrony. Specifically, while the relatively high proportions of EPI and embryonic nascent mesoderm coincides with the cell state composition of natural early to mid-gastrulating embryos (˜E6.5-7.0), the specification of extraembryonic mesoderm derivatives (i.e. chorion/amnion progenitors), caudal EPI, and definitive ectoderm frequencies resembles the composition of advanced gastrulating natural embryo stages (˜E7.0-7.5, FIG. 4D-FIG. 4F). Yet, the transcriptional profile of properly represented cell states was nearly identical to that of comparable cell states in natural embryos (FIG. 4G, FIG. 11C and FIG. 11D).

EPC lineage cells were absent from Day 6 EiTiX-embryoids compared to respective natural embryos. In contrast, the chorion lineage, comprising both chorion progenitors and their differentiated progenies, was largely intact (FIG. 4D, FIG. 4H, FIG. 4I and FIG. 11C). Gene expression analysis showed both programs to be overall highly similar to the natural embryos (FIG. 4J). It also revealed down-regulation of bona-fide chorion genes Rhox6 and Rhox9, together with upregulation of Id2, consistent with a lack of proximal signals emanating from the EPC compartment. Taken together, this analysis identified remarkably similar transcriptional states between Day 6 EiTiX-embryoids and their natural counterparts, but it also revealed pausing in mesoderm differentiation and over-accumulation of posterior cell types, most likely reflecting alterations in synchronicity between the mesoderm and ectoderm lineages. Therefore, whether further cultures of embryoids would enhance the synchronicity of lineage development was investigated next.

### Example 4

### EiTiX-Embryoids Develop to Late Headfold Stages with Heart and Chorion Development

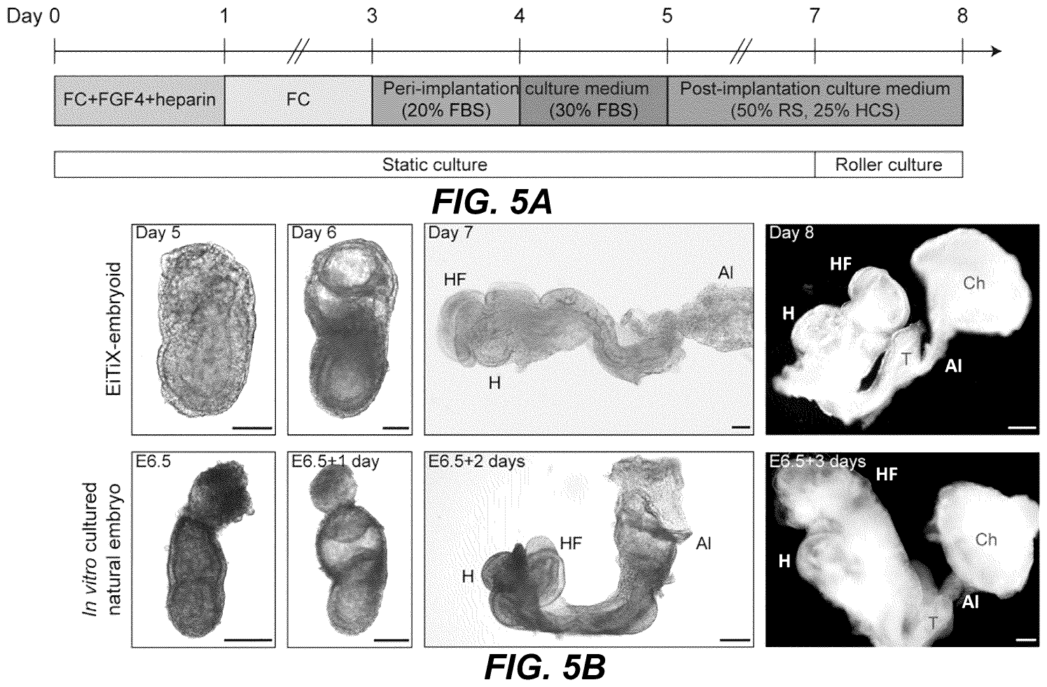

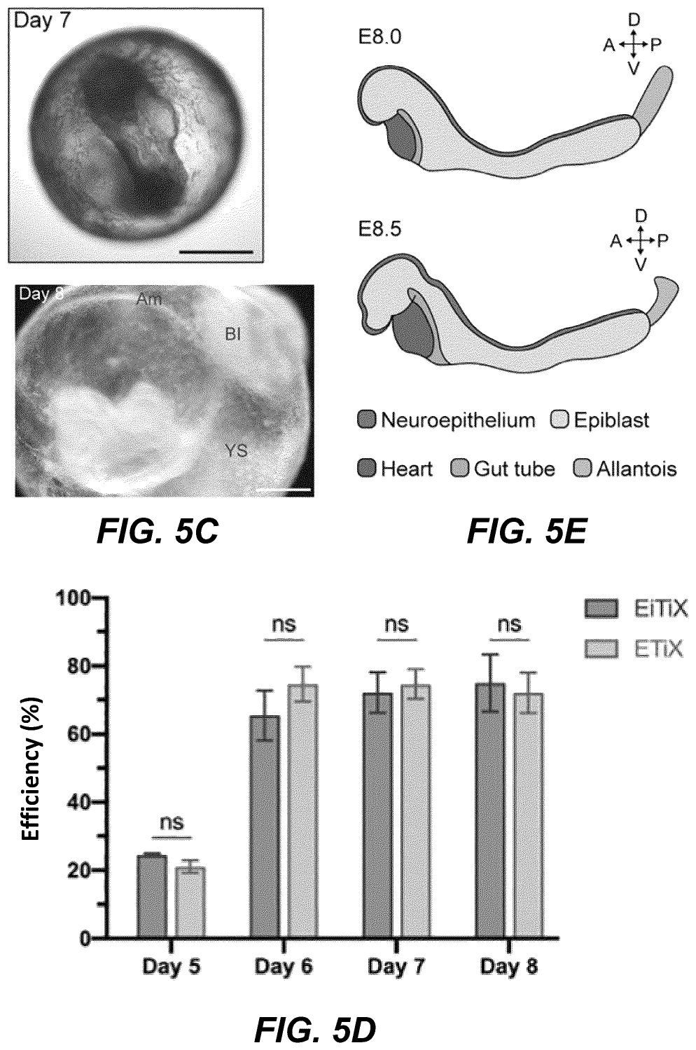

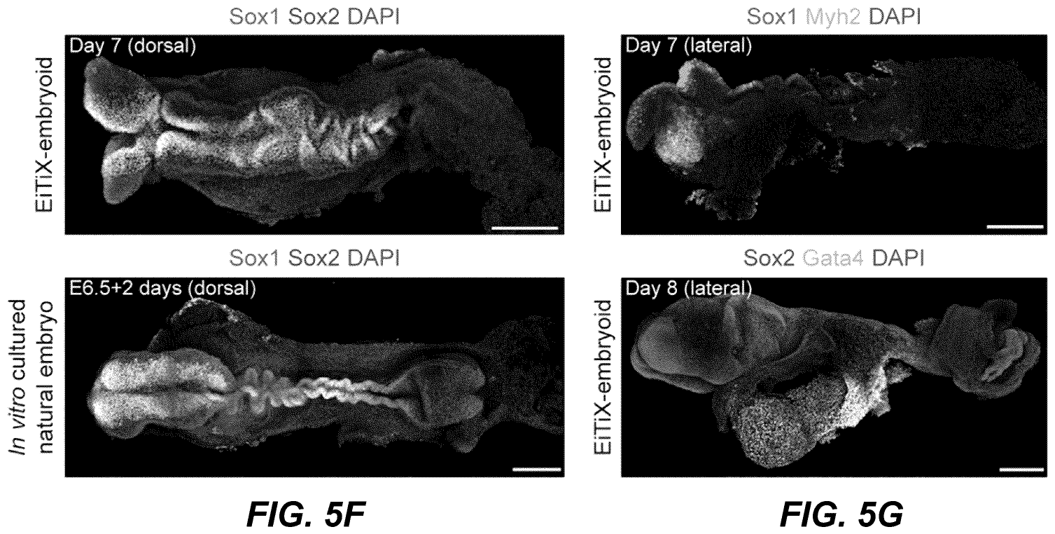

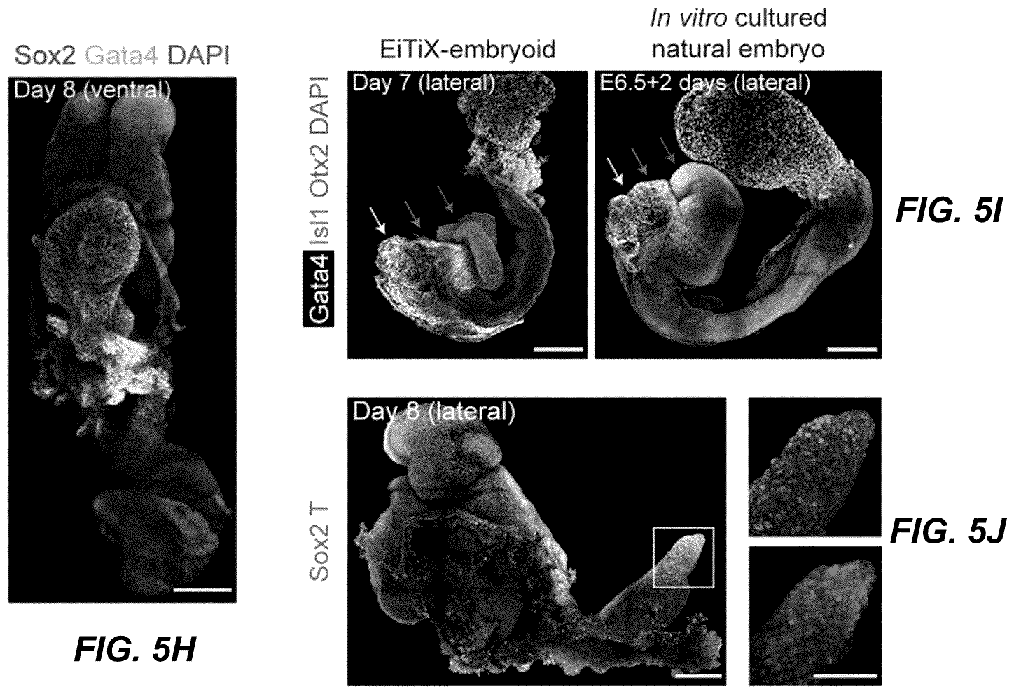

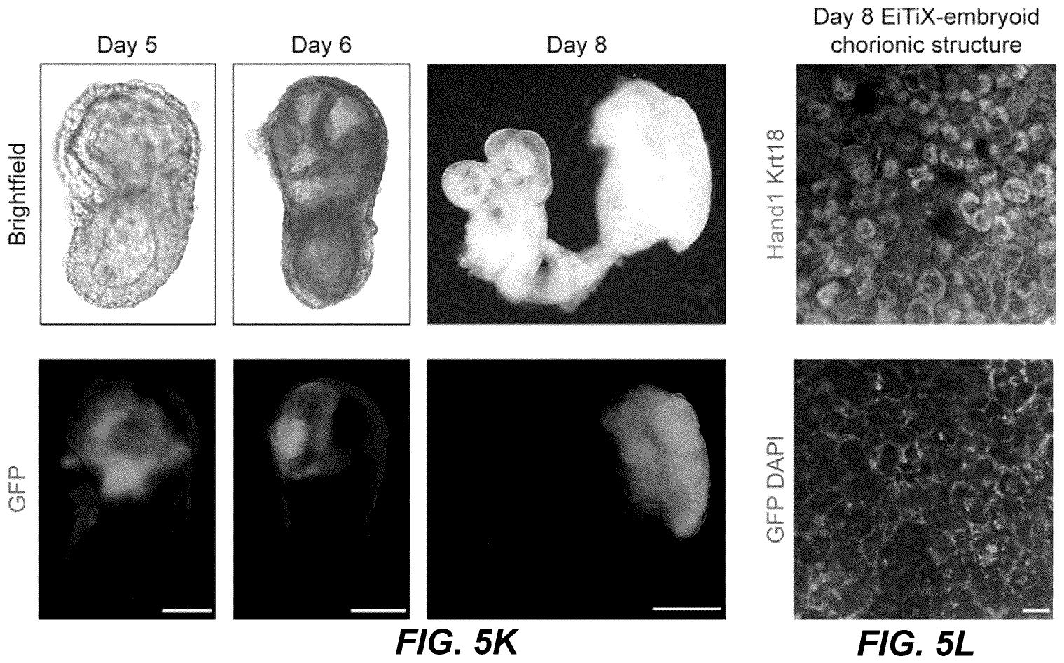

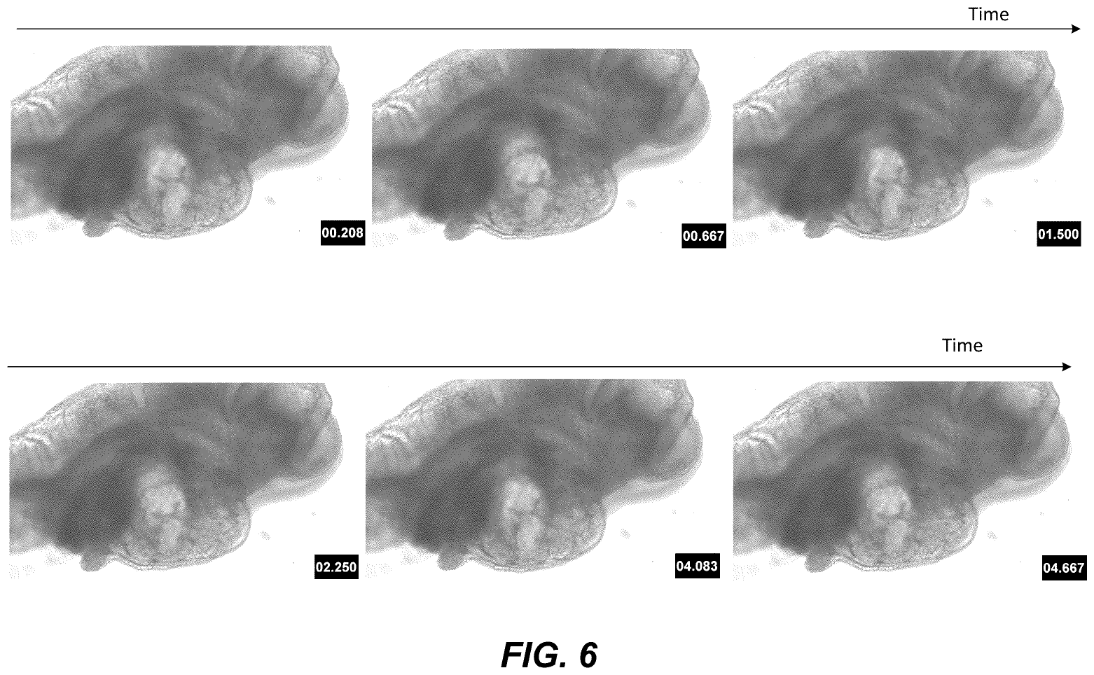

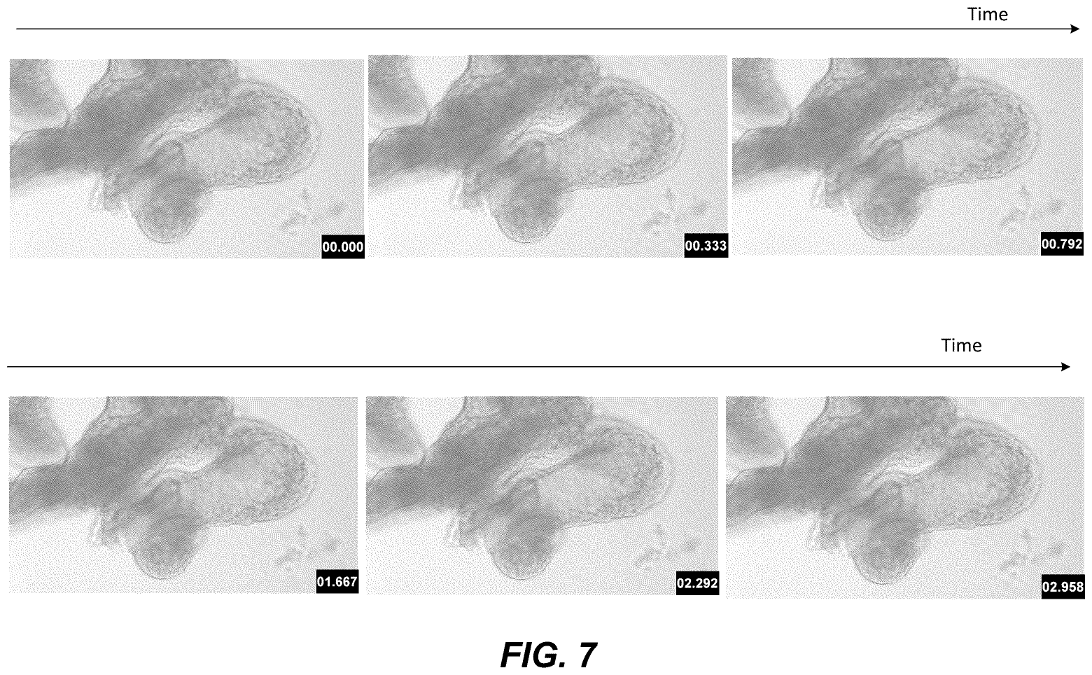

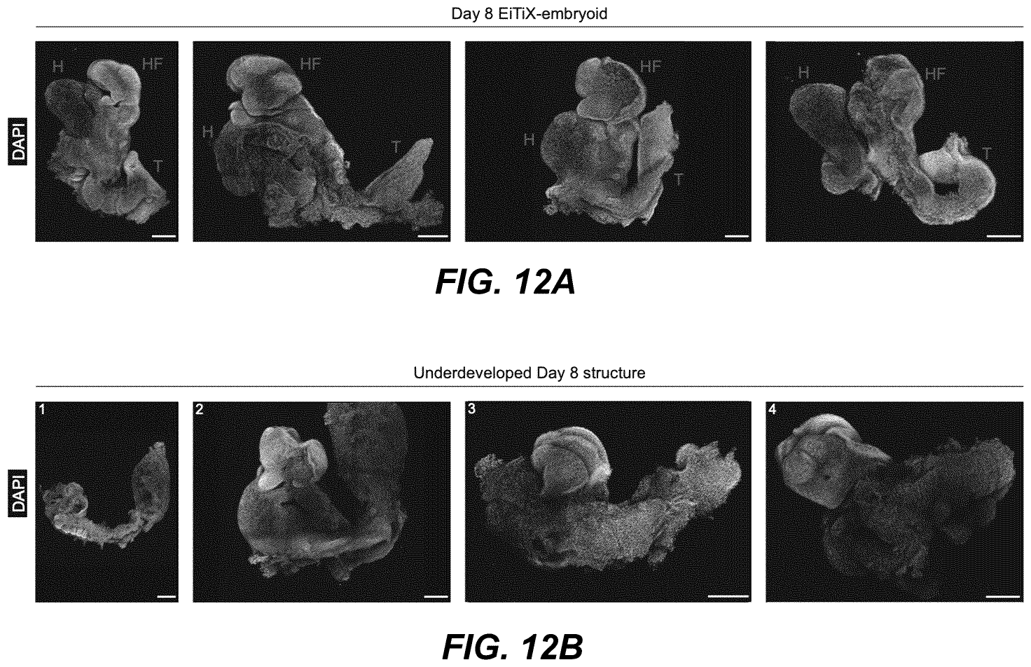

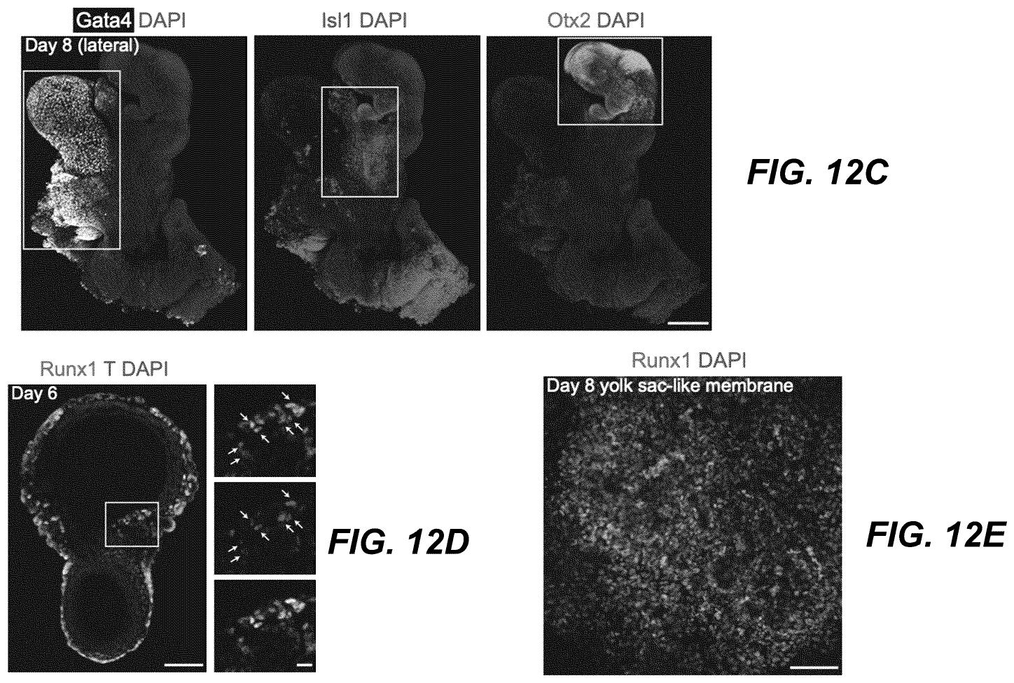

This example is carried out to assess the full developmental potential of EiTiX-embryoids. EiTiX-embryoids were transferred from peri-implantation to post-implantation culture medium (FIG. 5A). It was found that EiTiX-embryoids developed to establish headfolds, a beating heart, allantois, and chorion structures over the next 3 days in culture and they shared highly similar morphologies with E8.5 natural embryos cultured in vitro (FIG. 5B, FIG. 6 and FIG. 7). EiTiX-embryoids also developed amnion-like and yolk-sac-like membranes that properly enveloped the embryonic structures. Moreover, regions resembling blood islands, both in spatial localization and stereotypical red pigmentation, could be observed on the yolk-sac-like membrane (FIG. 5C). The efficiencies of EiTiX-embryoids progressing from Day 5 to 6, Day 6 to 7, and Day 7 to 8 were between 65.4% and 75%, which were comparable to that of ETiX-embryoids (FIG. 5D). Successfully developed Day 8 EiTiX-embryoids had well defined structures resembling headfolds, heart, and tail (FIG. 12A). The most commonly observed phenotypes of underdeveloped Day 8 EiTiX-embryoids include stunted overall development and impaired axial elongation to generate posterior structures (FIG. 12B).

Similar to natural E8.0 embryos and E6.5 embryos cultured in vitro for 2 days, the neuroepithelium markers Sox1 and Sox2 were expressed along the anterior-posterior axis of Day 7 EiTiX-embryoids (FIG. 5E and FIG. 5F), indicative of neurulation. Interestingly, twisting of the neural-tube-like region was observed in both Day 7 EiTiX-embryoid and in vitro cultured embryo, suggesting that this could be a defect of in vitro culture. The heart markers Myh2 and Gata4 were expressed below the headfolds (FIG. 5G), and a ventral view of the Gata4-expressing heart region revealed a morphology that resembled the linear heart tube (FIG. 5I). Importantly, the anterior region of the headfolds showed robust Otx2 expression, indicating development of the forebrain (FIG. 5I and FIG. 12C). The expression of Islet1 (Isl1), a pharyngeal mesoderm marker, as also detected between the Gata4-expressing heart region and Otx2-expressing forebrain region, recapitulating the expression pattern in the in vitro cultured embryos (FIG. 5I and FIG. 12C). At the posterior end of the body axis, robust co-expression of Sox2 and T was observed at the region resembling the tail, which identified the neuromesodermal progenitor population (FIG. 5J).

As the neurulating EiTiX-embryoids arise from ESC and two different types of induced extraembryonic ESC types, the extent of extraembryonic tissue development was determined. Chorion, an ExE-derived tissue that forms part of the placenta, and chorion progenitors were detected in Day 6 EiTiX-embryoids by scRNA-seq (FIG. 4D) and could observe a region that resembled the chorion in Day 8 EiTiX-embryoids (FIG. 5K). To ask whether this region was derived from iCdx2 ESCs, Day 8 EiTiX-embryoids were generated using iCdx2 ESCs constitutively expressing membrane-associated GFP combined with unlabeled WT and iGata4 ESCs. Membrane-associated GFP was observed in the ExE region in Day 5 and Day 6 EiTiX-embryoids. On Day 8, the membrane GFP was exclusively found in the region resembling the chorion (FIG. 5K). Further examination of this latter region showed co-expression of the chorion markers Hand1 and Keratin18 (FIG. 5L).

Another crucial extraembryonic cell type is the extraembryonic mesoderm, which contributes to amnion and yolk sac in the natural mouse embryo. In Day 6 EiTiX-embryoids, cells expressing both T, a mesoderm marker, and Runx1, a hematopoietic marker that is enriched in extraembryonic mesoderm could be observed in the region resembling the proximal EPI (FIG. 12D), suggesting the emergence of extraembryonic mesoderm. Runx1 is also a key transcription factor driving definitive hematopoiesis and is expressed in blood islands. As blood islands had been observed on the yolk-sac-like membrane in Day 8 EiTiX-embryoids (FIG. 5C), the yolk-sac-like membrane was dissected out and robust Runx1 expression could be detected, indicative of extraembryonic hematopoiesis (FIG. 12E).

Taken together, these results indicate that EiTiX-embryoids have the remarkable ability to develop to headfold stages. They not only gave rise to advanced embryonic structures such as neuroepithelium, a beating heart, and mesodermal populations, but importantly, also developed extraembryonic tissues including yolk sac and chorion. The tracking of membrane-GFP-positive iCdx2 ESCs further confirmed that iCdx2 ESCs could effectively develop into chorion, an ExE-derived tissue, demonstrating that an embryoid could be generated with embryonic and extraembryonic tissues entirely from ESCs.

### Example 5

### Cell State and Composition Analysis of Neurulating Embryoids Using scRNA-Seq

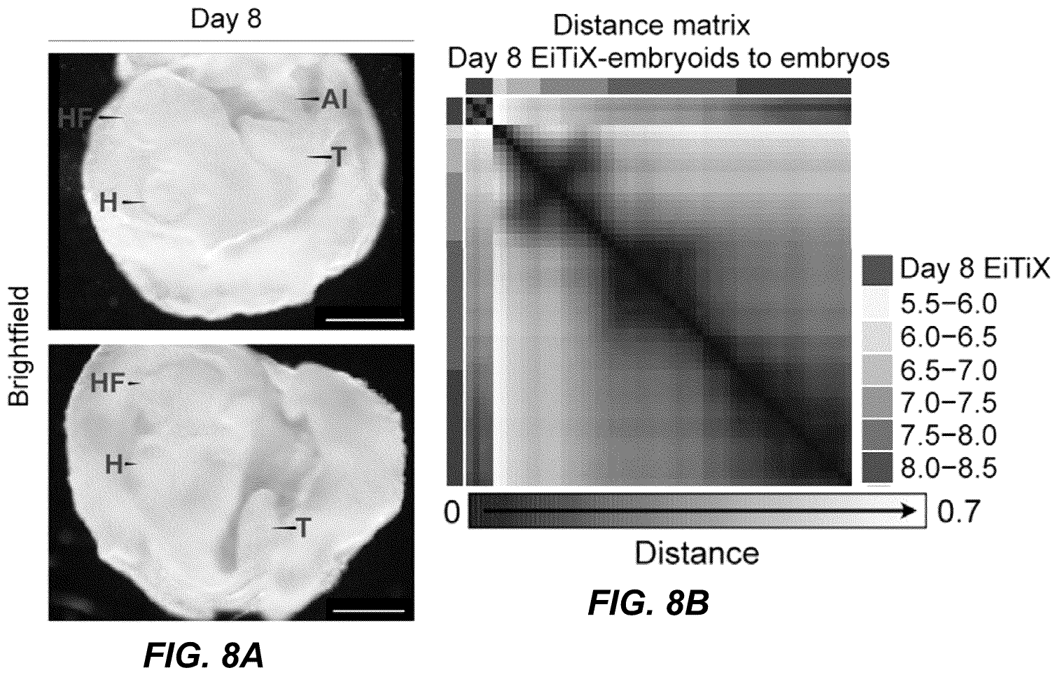

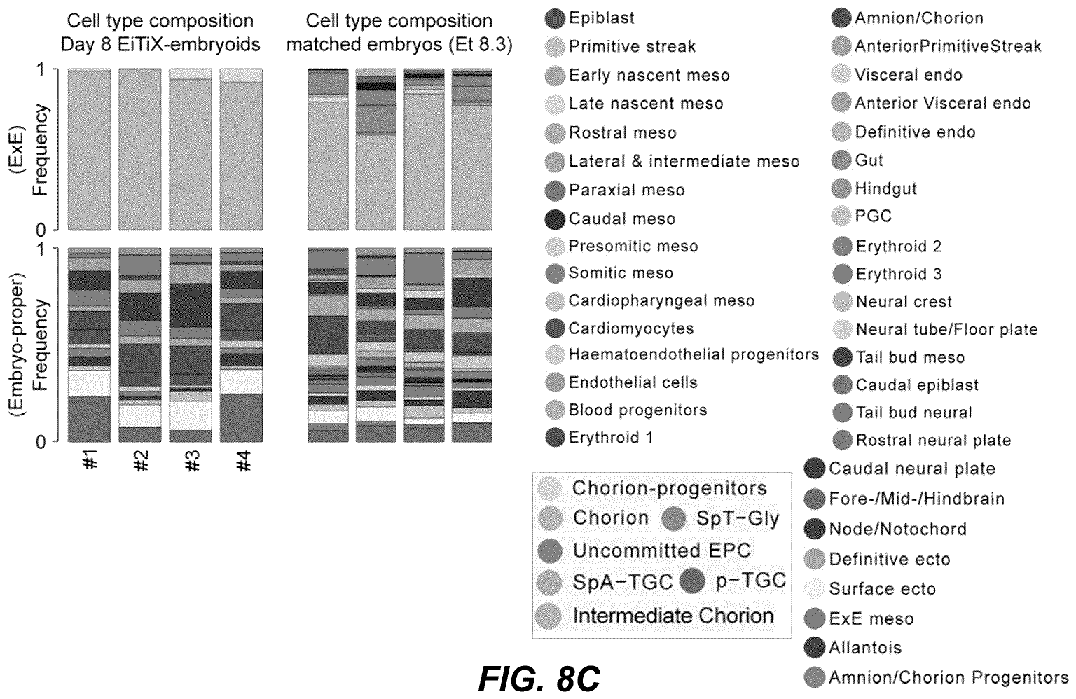

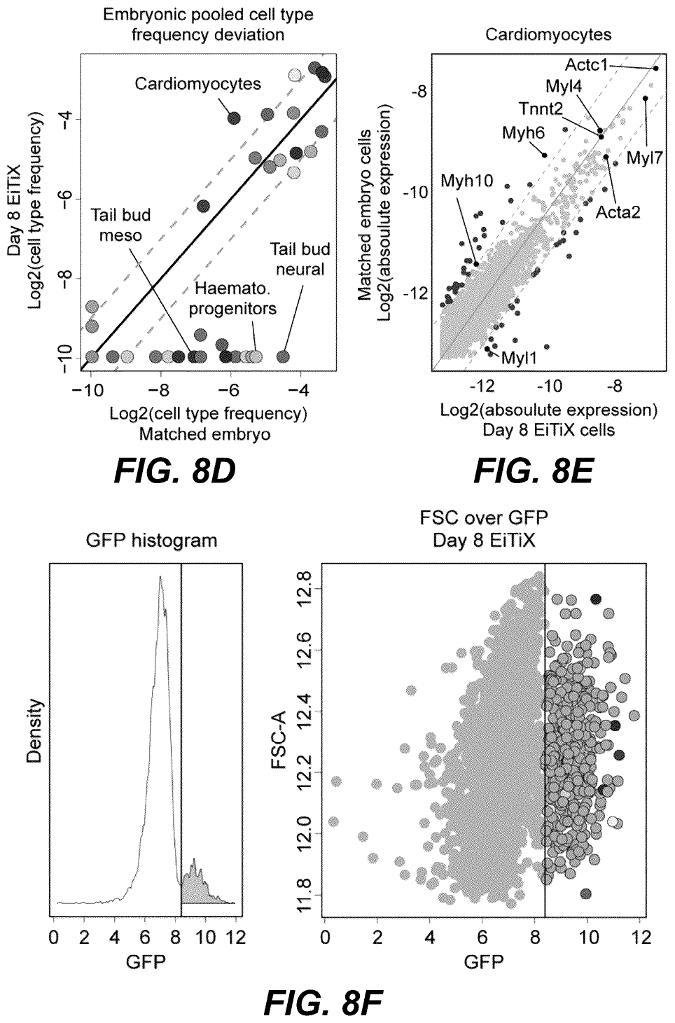

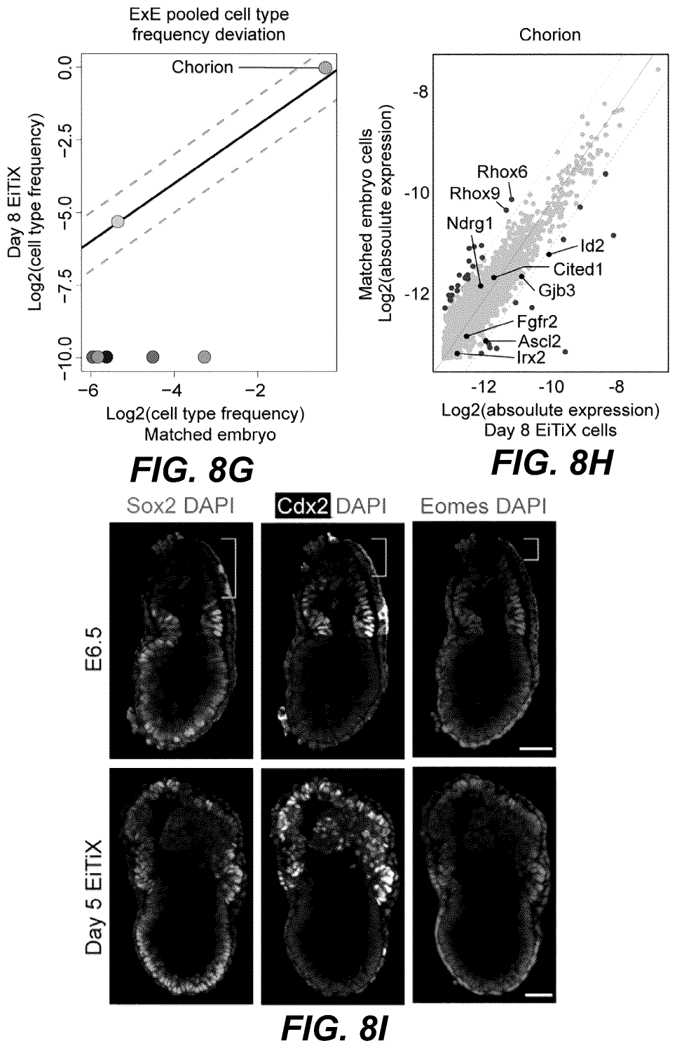

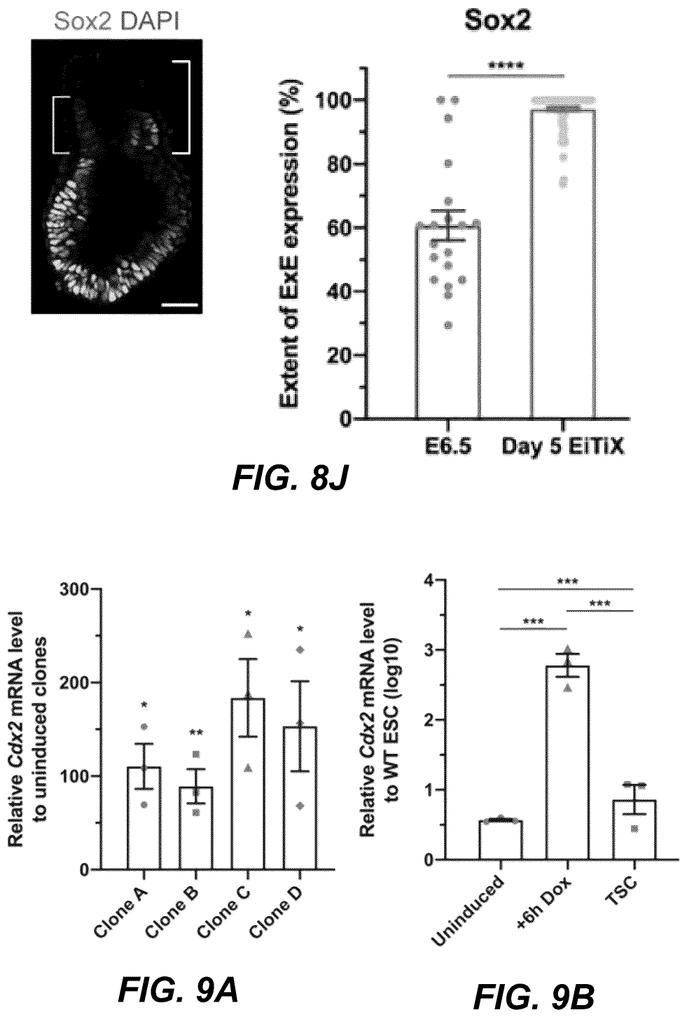

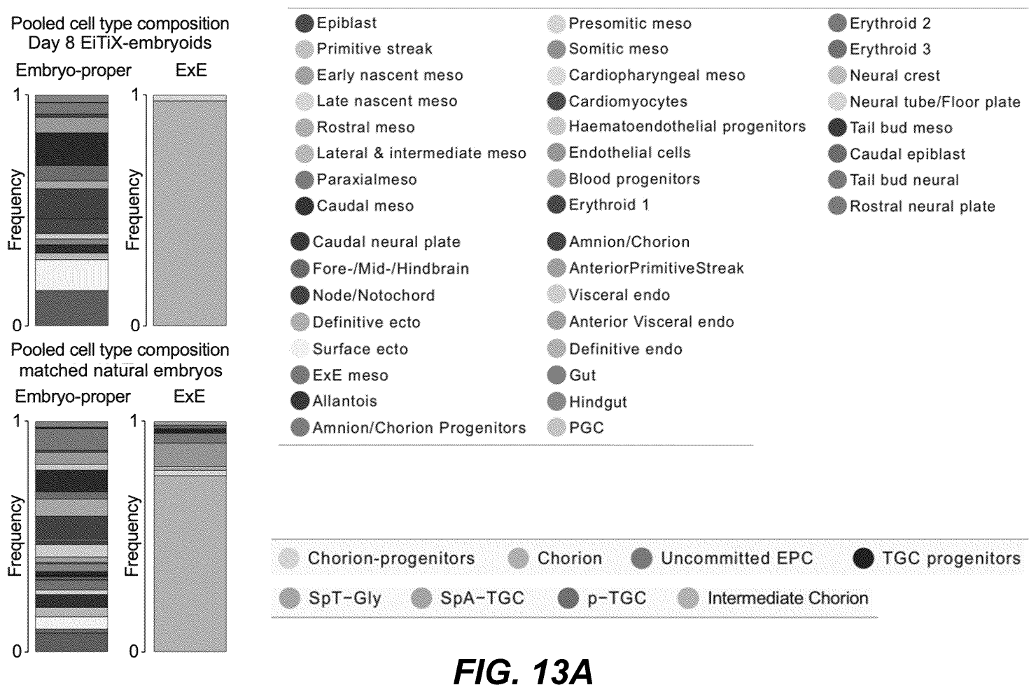

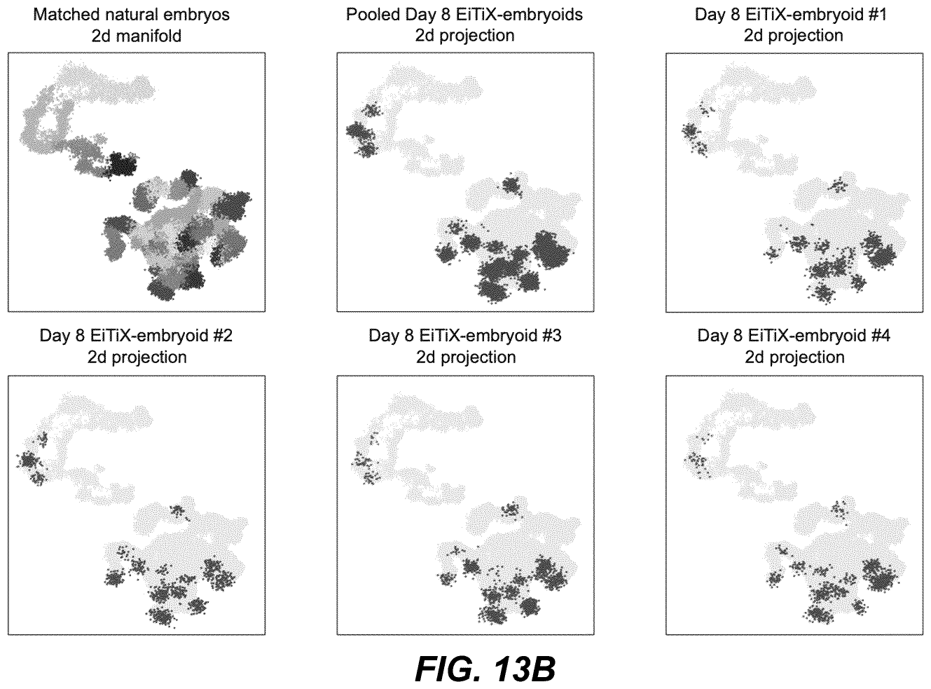

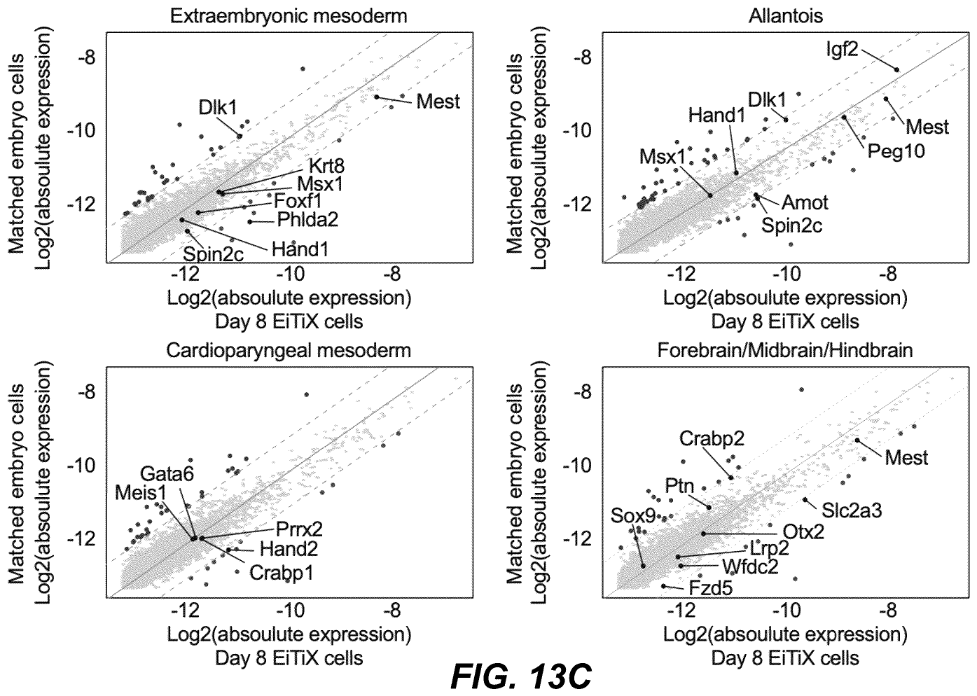

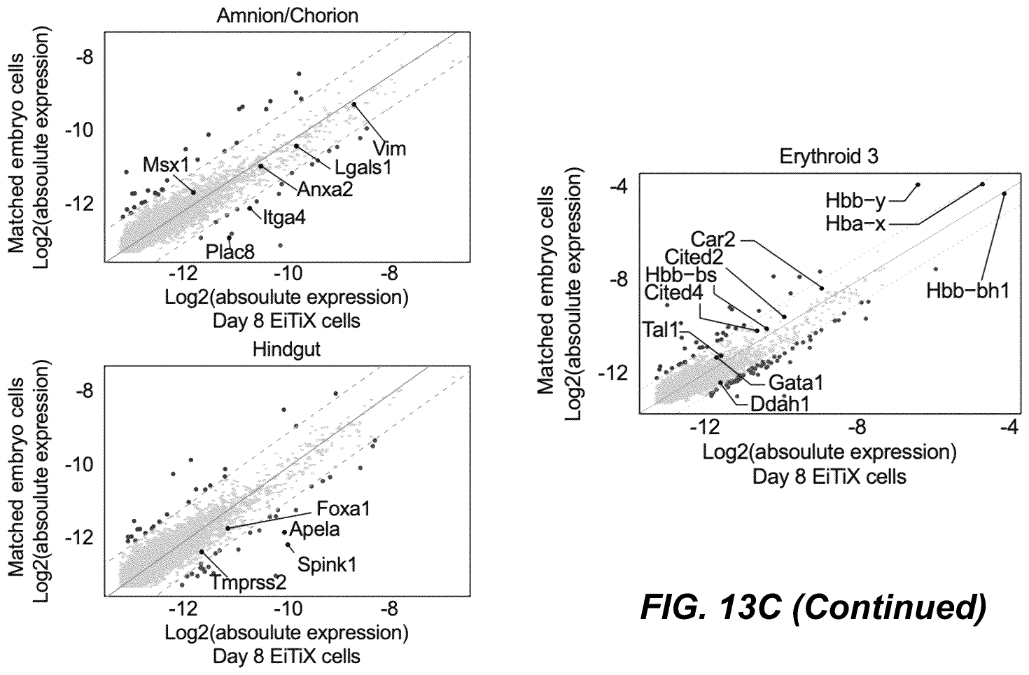

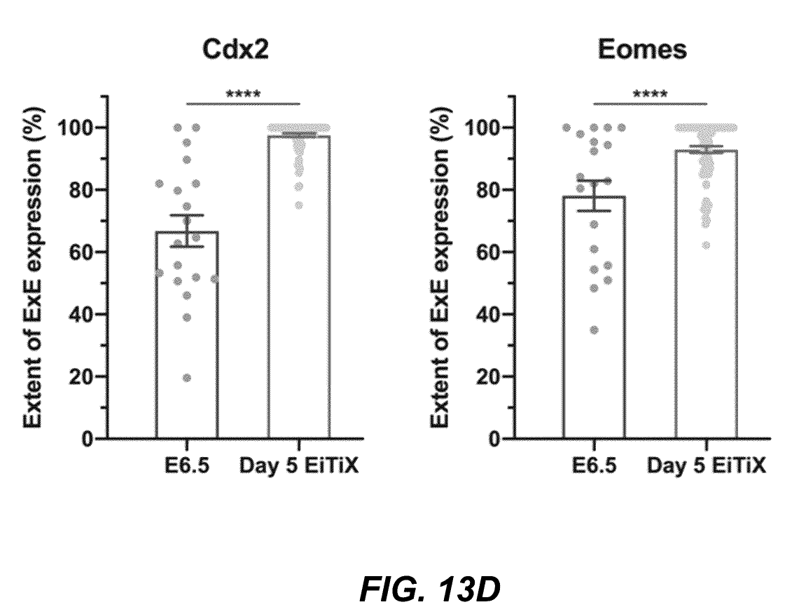

To undertake a comprehensive analysis of cell state integrity and composition in neurulating embryoids, four Day 8 EiTiX-embryoids were collected for scRNA-seq (FIG. 8A). Transcriptional similarity analysis showed that Day 8 EiTiX-embryoids overall most resembled E8.0-8.5 stages (FIG. 8B). Analysis of cell state composition confirmed the high similarity between individual embryoids. In addition, Day 8 embryoids displayed high synchronicity between lineages and exhibited advanced cell states consistent with late headfold stages (FIG. 8C, FIG. 13A and FIG. 13B). Quantitative analysis of cell-state frequency deviations identified depletion of tail bud cell types and hematoendothelial progenitors. Furthermore, an over-representation of cardiomyocyte cell state (FIG. 8D) was found, which may correspond to the apparent larger cardiac domain (FIG. 12A). These deviations must be viewed cautiously, given the low frequency associated with some of these cell types. For example, although presomitic mesoderm was not present in Day 8 EiTiX-embryoids, a bona-fide somitic mesoderm population could be detected, suggesting that this progenitor population was merely not sampled. Finally, similar gene expression patterns were detected in high-frequency cell states compared to controls (FIG. 8E and FIG. 13C).

Analyzing ExE differentiation showed expected progression in the chorion lineage (i.e. most chorion progenitors fully converting to their differentiated progenies). However, any cell types associated with the EPC lineage could not be detected, including uncommitted EPCs and trophoblast giant cell (TGC) progenitors (FIG. 8C and FIG. 13A). The vast majority of GFP-positive iCdx2 ESCs (97.26%) gave rise to chorion lineage, although a few embryonic cell types were noted among the GFP-positive cells (FIG. 8F). Indeed, GFP-positive cells were occasionally observed in the EPI-like compartment in Day 4 EiTiX-embryoids, and these cells might have retained ESC fate and would eventually give rise to embryonic lineages. Overall, cells in the chorion lineage appeared with comparable frequencies and exhibited highly similar gene expression signatures when compared to time-matched natural embryos (FIG. 8G and FIG. 8H). The inability to detect EPC and TGC subtypes in both Day 6 and Day 8 EiTiX-embryoids suggests that iCdx2 ESCs exhibited restricted ExE differentiation potential. In the natural embryo, the ExE can be subdivided into proximal ExE (adjacent to the EPI/ExE boundary) and distal ExE (toward the tip of the embryo). The proximal ExE is characterized by the expression of TSC-like markers such as Sox2, Cdx2, and Eomes, which are downregulated in the distal ExE, where the EPC and TGC subtypes are found. The absence of TSC-like markers in the distal ExE in E6.5 embryos (bracket) was confirmed, whereas in Day 5 EiTiX-embryoids, strong expression of these genes was observed throughout the ExE-like compartment (FIG. 8I), with an extended-expression domain (FIG. 8J and FIG. 13D).

### Additional Consideration

The above examples demonstrate that ESCs carrying a Cdx2 transgene can adopt a TSC-like cell fate upon Dox-induced Cdx2 overexpression. The resulting iCdx2 ESCs had the ability to self-assemble with ESCs induced to overexpress Gata4 (induced XEN cells) and WT ESCs to generate an in vitro model of mouse post-implantation development with embryonic and extraembryonic lineages. The embryonic-extraembryonic embryo model, term “EiTiX-embryoids,” was derived entirely of ESCs and thus circumvented the use of undefined media to culture conventional extraembryonic cell lines. When EiTiX-embryoids were cultured in the peri-implantation culture medium in stationary culture, they specified the DVE and AVE, establish an anterior-posterior axis, and underwent gastrulation. Following transfer into roller-bottle system and enriched post-implantation culture medium, EiTiX-embryoids undertook neurulation; formed headfolds, brain, and a beating heart structure; and developed extraembryonic tissues including yolk sac and chorion.

Single-structure scRNA-seq of 14 Day 6 and 4 Day 8 EiTiX-embryoids were performed and the data was projected on a temporal model describing the parallel differentiation in embryonic and extraembryonic lineages. This enabled direct comparisons with time-matched natural embryos, providing an analytical framework for quantifying the fidelity of intracellular transcriptional programs and overall cell composition within individual structures. Overall similarity of Day 6 EiTiX-embryoids with natural embryos of E6.0 to E7.5 stages was observed, whereas Day 8 EiTiX-embryoids were most similar to natural embryos from E8.0 to E8.5. Despite the morphological variability of EiTiX-embryoids, transcriptional states appeared remarkably conserved compared to corresponding ones in natural embryos. Two main types of deviation from the natural flow of the embryo proper were noted: (1) some differentiated cell types were missing in both Day 6 and Day 8 embryoids, and (2) synchronicity between lineages was impaired in Day 6 embryos. Nevertheless, adaptations in culture conditions significantly improved lineage synchronicity in Day 8 embryoids, resulting in much more comparable cell compositions to that of time-matched embryos. The analytical approach described here can complement future screening aimed at improved culture conditions by providing a robust quantitative readout on embryoid development.

The EiTiX-embryoid was thus able to develop many more tissues than structures derived solely from homogeneous populations of ESCs induced to differentiate by various exogenous molecules, generating a more complete in vitro model with both embryonic and extraembryonic tissues. Hence, the data described hereby substantiate the essential role of extraembryonic tissues in driving the self-organization of mouse-embryo-like structures. For example, the role of ESCs induced to express Gata4 was of critical importance in establishing the formation of the AVE, which was required to direct the formation of anterior structures, particularly such as those of the forebrain.

Although the induced extraembryonic structures contributed to the correct development of diverse embryonic cell types and overall structure in EiTiX-embryoids, the development of the ExE lineage was incomplete as reflected by the lack of EPC and TGC cell types. This could be partly because EiTiX-embryoids lack the interactions with the maternal environment that they would have in utero, and partly because transcription-factor-mediated induction biases iCdx2 ESCs to differentiate into chorionic cell types. It is possible that there are two types of progenitor cells in the ExE splitting immediately after implantation and iCdx2 ESCs resemble most the chorion lineage progenitors. There was a strong and extended expression of TSC-like markers throughout the ExE-like compartment in Day 5 EiTiX-embryoids, unlike the ExE in E6.5 natural embryos. Thus, it might be necessary to introduce the expression of genes promoting ExE differentiation or induce the downregulation of TSC-like genes to generate EPC and TGC subtypes in EiTiX-embryoid. Moreover, by incorporating TE-derived cell types, this system offered future possibilities for dissecting the precise roles of such cells in the developmental process.

Advanced development of ETiX-embryoids, which used TSCs, unmodified ESCs, and Gata4 inducible ESCs, to late headfold stages has recently been reported. Interestingly, ETiX-embryoids also lack TGCs and spongiotrophoblasts, which could again be attributed to the lack of maternal interaction. EiTiX-embryoids and ETiX-embryoids share similar efficiency to progress to late headfold stages and that they undergo similar development of major tissues, including embryonic tissues such as headfolds and heart, and extraembryonic tissues such as allantois, chorion, and yolk sac blood island. Nevertheless, ETiX-embryoids capture more cell types than EiTiX-embryoids. For example, PGCs could be detected near the allantois in Day 7 and Day 8 ETiX-embryoids but not in Day 8 EiTiX-embryoids. This could reflect differences in signaling molecules secreted by TSCs compared to iCdx2 ESCs.

Another group has also recently reported the generation of embryoids from mouse ESCs adapting a previously described protocol and using a similar induction approach, although with a much longer induction period (1-14 days pre-aggregation plus 2 days post-aggregation as compared to 6 h pre-aggregation only here). Similar to the EiTiX-embryoids, their embryoids reached a developmental stage resembling E8.5 embryos and did not develop further. The absence of some TGC and spongiotrophoblast markers was also noted in the embryoids from mouse ESCs that is similar to EiTiX-embryoids.

Despite not recapitulating the later stages of development of extraembryonic tissues and their lack of some cell types present in ETiX-embryoids, the substitution of TSCs by iCdx2 ESCs in EiTiX-embryoid permitted remarkable development of the embryo per se, with the development of a yolk sac and chorion. The reconstitution of the three principal lineages of peri-implantation development exclusively from ESCs ensured simplified, defined, and consistent culture conditions to recapitulate the interactions between embryonic and extraembryonic tissues that facilitated development through gastrulation to neurulation-like stages.

The Examples above have shown that iCdx2 ESCs could replace TSCs to generate embryoids, but the extent of their contribution to ExE lineages in natural embryos was not tested. However, it has been reported that Cdx2-overexpressing ESCs could contribute to the placenta of E12.5 chimeric embryos. Secondly, the level of Cdx2 overexpression is critical to successful generation of EiTiX-embryoids. The generation of EiTiX-embryoids has been tested using the clone of iCdx2 ESCs that had the lowest level of Cdx2 overexpression (FIG. 9A, clone B) and found the efficiency of obtaining organized structures on Day 4 was very low (approximately 3%), which could be due to insufficient reprogramming of iCdx2 ESCs into TSC-like cells. Furthermore, EiTiX-embryoids generated using different ESC lines had different developmental potential. EiTiX-embryoids generated using a combination of CD1 unmodified ESCs, CD1 Gata4 inducible ESCs, and CAG-GFP Cdx2 inducible ESCs could consistently undergo advanced development to late headfold stages. Lastly, the efficiencies of EiTiX-embryoids progressing to Day 4 and Day 5 were only 15.5% and 24.4%, respectively. However, by setting up several AggreWells at the start of the experiment, around 350 well-developed Day 4 EiTiX-embryoids would routinely be obtained per experiment, which exceeded the capacity to transfer all of them to post-implantation culture conditions due to the costs and availability of serum for culture media.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of generating a synthetic embryo in vitro, the method comprising:
(a) co-culturing a wild-type mammalian embryonic stem cell (ESC), a mammalian ESC overexpressing CDX2 transcription factor, and a mammalian ESC overexpressing GATA transcription factor under a condition allowing the ESCs to self-assemble into a gastrulating embryo structure; and
(b) culturing the gastrulating embryo structure in a post-implantation culture medium under a condition allowing the gastrulating embryo structure to develop into a synthetic embryo.

2. The method of claim 1, the GATA transcription factor is GATA4.

3. The method of claim 1, further comprising:
generating the mammalian ESC overexpressing GATA transcription factor and the mammalian ESC overexpressing CDX2 transcription factor by contacting a mammalian ESC carrying an inducible Gala gene and a mammalian ESC carrying an inducible Cdr2 gene with an inducer.

4. (canceled)

5. (canceled)

6. The method of claim 1, wherein the ESCs are co-cultured for up to 6 days.

7. The method of claim 1, wherein the gastrulating embryo structure resembles an E6.0-E7.5 natural embryo structure, optionally an E6.0-E6.5, E6.5-E7.0, or E7.0-E7.5 natural embryo structure.

8. The method of claim 1, wherein the ESCs are cultured in a substrate, wherein the substrate comprises a dish, a U-plate, a flask or a microwell plate.

9. (canceled)

10. (canceled)

11. The method of claim 1, wherein step (a) comprises culturing the ESCs in a feeder cell (FC) media for about 3 days, and wherein step (a) further comprises culturing the ESCs in an in vitro culture (IVC) media for about 2 days.

12.-14. (canceled)

15. The method of claim 11, wherein the FC media and the IVC media comprise a basal culture medium.

16. The method of claim 15, wherein the basal culture medium comprises Dulbecco's Modified Eagle Media (DMEM), DMEM Nutrient Mixture 12 (DMEM/F12), a non-human serum or serum substitute thereof, a reducing agent, an antibiotic, L-glutamine or an analogue thereof, or any combination thereof, wherein
the non-human serum or serum substitute comprises fetal bovine serum, bovine serum albumin, KnockOut™ Serum Replacement, or any combination thereof;
the reducing agent comprises beta-mercaptoethanol (BME), N-acetyl-L-cysteine, dithiothreitol (DTT), or any combination thereof; and/or
the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof.

17.-21. (canceled)

22. The method of claim 11, wherein the FC media comprises DMEM, fetal bovine serum, sodium pyruvate, GlutaMax, MEM non-essential amino acids, beta-mercaptoethanol, penicillin and/or streptomycin, or any combination thereof; and/or wherein the IVC media comprises DMEM/F12, fetal bovine serum, GlutaMax, ITS-X, β-estrodiol, progesterone, N-acetyl-L-cysteine, penicillin and/or streptomycin, or any combination thereof.

23. (canceled)

24. The method of claim 11, wherein the FC media further comprises an anticoagulant, optionally heparin, a fibroblast growth factor (FGF), optionally FGF2 and/or FGF4, or any combination thereof; and/or the FC media is supplemented with heparin, Fgf4, and/or ROCK inhibitor.

25. (canceled)

26. The method of claim 11, wherein the IVC media comprises:
a) insulin, an insulin analogue, or an insulin receptor agonist;
b) estrogen, an estrogen analogue, or an estrogen receptor agonist; and
c) progesterone, a progesterone analogue, or a progesterone receptor agonist, wherein
the insulin receptor agonist is selected from the group comprising IGF-I, IGF-II, analogues thereof, or any combination thereof; and/or
the estrogen receptor agonist is selected from the group comprising β-estradiol, estrone, estriol and estetrol, or any analogue thereof.

27.-32. (canceled)

33. The method of claim 1, wherein co-culturing the ESCs comprises increasing serum concentrations, optionally increasing the serum concentration from about 20% to about 30%, optionally when the ESCs are co-cultured in the IVC media.

34. (canceled)

35. The method of claim 1, wherein step (a) is performed under a static condition and/or wherein step (b) is for a duration of at least 3 days.

36. (canceled)

37. The method of claim 1, wherein the post-implantation culture media is capable of supporting the development of embryo ex utero.

38. The method of claim 1, wherein the post-implantation culture media comprises DMEM, non-human serum, human cord serum, L-glutamine or an analogue thereof, antibiotics, or any combination thereof, and wherein the non-human serums comprises rat and/or bovine serum.

39. (canceled)

40. The method of claim 38, wherein the post-implantation culture medium comprises bicarbonate and/or HEPES.

41. (canceled)

42. (canceled)

43. The method of claim 1, wherein the post-implantation culture medium comprises DMEM, rat serum, human cord serum, GlutaMax, penicillin and/or streptomycin, HEPES, or any combination thereof.

44. (canceled)

45. The method of claim 1, wherein step (b) comprises culturing the gastrulating embryo structure under a dynamic condition in a culture chamber, following culturing the gastrulating embryo structure under a static condition.

46. The method of claim 45, wherein culturing the gastrulating embryo structure under the static condition is for a duration of about 2 days and/or wherein culturing the gastrulating embryo structure under the dynamic condition is for a duration of at least one day.

47. (canceled)

48. The method of claim 45, wherein the dynamic condition comprises suspension agitation, and wherein culturing the gastrulating embryo structure under the dynamic condition is performed in a rotating bottle culture chamber.

49. (canceled)

50. The method of claim 48, wherein the rotating bottle culture chamber contains up to 3 synthetic embryoid in about 2 ml post-implantation medium.

51. The method of claim 1, wherein step (b) comprises supplying the post-implantation culture medium with glucose, optionally with at least 3 mg/ml glucose, and optionally when culturing under a dynamic condition.

52. The method of claim 1, wherein the synthetic embryo structure (1) resembles an E8.0-E8.5 natural embryo structure; (2) is a neutralizing embryo structure; (3) has established headfolds, a beating heart, allantois, chorion structure, and/or yolk sac; and/or (4) has developed amnion-like and yolk-sac-like membranes.

53.-55. (canceled)

56. The method of claim 1, wherein (1) the method does not comprise any in vivo step; (2) none of the wild-type ESC, the ESC overexpressing CDX2 transcription factor and the ESC overexpressing GATA transcription factor is present in an in vivo environment during step (a) or step (b); and optionally wherein the in vivo environment comprises a tissue, an organ, an organism, or a combination thereof; and/or (3) the method does not comprise culturing an extra-embryonic stem cell, optionally, the extra-embryonic stem cell comprises an extra-embryonic trophoblast stem cell and/or an extra-embryonic endoderm stem cell.

57. (canceled)

58. (canceled)

59. The method of claim 1, wherein the synthetic embryo is a mouse embryo.

60. A synthetic embryo obtained by the method of claim 1.

61.-69. (canceled)


SEQUENCE LISTING (10 sequences)

SEQ ID NO: 1 - DNA, 55 bp, synthetic construct
  source 1..55: mol_type = other DNA
ggggacaagtttgtacaaaaaagcaggcttaatgtacgtgagctaccttctggac

SEQ ID NO: 2 - DNA, 57 bp, synthetic construct
  source 1..57: mol_type = other DNA
ggggaccactttgtacaagaaagctgggtttcactgggtgacagtggagtttaaaac

SEQ ID NO: 3 - DNA, 19 bp, synthetic construct
  source 1..19: mol_type = other DNA
aaacctgtgcgagtggatg

SEQ ID NO: 4 - DNA, 20 bp, synthetic construct
  source 1..20: mol_type = other DNA
tctgtgtacaccacccggta

SEQ ID NO: 5 - DNA, 21 bp, synthetic construct
  source 1..21: mol_type = other DNA
atttctacagtccgctggtgc

SEQ ID NO: 6 - DNA, 22 bp, synthetic construct
  source 1..22: mol_type = other DNA
acatcaccgtgaagacaagtgg

SEQ ID NO: 7 - DNA, 20 bp, synthetic construct
  source 1..20: mol_type = other DNA
cgtattgggcgcctggtcac

SEQ ID NO: 8 - DNA, 20 bp, synthetic construct
  source 1..20: mol_type = other DNA
atgatgacccttttggctcc

SEQ ID NO: 9 - DNA, 20 bp, synthetic construct
  source 1..20: mol_type = other DNA
tgtctgcgaacactgagctg

SEQ ID NO: 10 - DNA, 22 bp, synthetic construct
  source 1..22: mol_type = other DNA
cgatcacctgagtagcaaggag